(19) United States
(12) Patent Application Publication
(10) Pub. No.: US 2025/0144211 A1
(43) Pub. Date: May 8, 2025

(54) RECOMBINANT PROTEINS THAT STIMULATE AN IMMUNE RESPONSE IN THE PRESENCE OF NATURALLY INHIBITORY LIGAND BINDING

(71) Applicant: SEATTLE CHILDREN'S HOSPITAL D/B/A SEATTLE CHILDREN'S RESEARCH INSTITUTE, Seattle, WA (US)

(72) Inventor: Shannon Oda, Brier, WA (US)

(73) Assignee: Seattle Children's Hospital d/b/a Seattle Children's Research Institute, Seattle, WA (US)

(21) Appl. No.: 18/836,523

(22) PCT Filed: Feb. 7, 2023
(86) PCT No.: PCT/US23/62159

§ 371 Date: Aug. 7, 2024

Related U.S. Application Data
(60) Provisional application No. 63/307,586, filed on Feb. 7, 2022.
(60) Provisional application No. 63/307,574, filed on Feb. 7, 2022.

(51) Int. Cl. A61K 40/30; A61K 40/11; A61K 40/40

(52) U.S. Cl. CPC A61K 40/30; A61K 40/11; A61K 40/40

(57) ABSTRACT

Recombinant proteins that stimulate an immune response in the presence of naturally inhibitory ligand binding are described. The recombinant proteins include an extracellular domain of an inhibitory immune cell protein and an intracellular domain of a stimulatory immune cell protein connected via a transmembrane domain. The recombinant proteins can be used to stimulate immune cell activation in the fight against cancers and infectious diseases, among other uses.

### CROSS-REFERENCE TO RELATED APPLICATIONS

This application is a U.S. National Phase Application based on International Patent Application No. PCT/US2023/062159, filed Feb. 7, 2023, which claims priority to U.S. Provisional Patent Application No. 63/307,574 filed Feb. 7, 2022 and to U.S. Provisional Patent Application No. 63/307,586 filed Feb. 7, 2022, the contents of all of which are incorporated by reference herein in their entirely.

### REFERENCE TO SEQUENCE LISTING

The Sequence Listing associated with this application is provided in text format in lieu of a paper copy, and is hereby incorporated by reference into the specification. The name of the text file containing the Sequence Listing is 39T2391.XML_ST26.xml. The text file is 118,784 bytes, was created on Jul. 24, 2024, and is being submitted electronically via Patent Center.

### FIELD OF THE DISCLOSURE

The current disclosure provides recombinant proteins that stimulate an immune response in the presence of naturally inhibitory ligand binding. The recombinant proteins include an extracellular domain of an inhibitory immune cell protein and an intracellular domain of a stimulatory immune cell protein connected via a transmembrane domain. The recombinant proteins can be used to stimulate immune cell activation in the fight against cancers and infectious diseases, among other uses.

### BACKGROUND OF THE DISCLOSURE

The immune system uses two general mechanisms to protect the body against cancerous cells or environmental pathogens such as viruses, bacteria, and fungi. One is the non-specific (or innate) inflammatory response. The other is the specific (acquired or adaptive) immune response. In contrast to the innate response which is fundamentally the same for each injury, acquired responses are custom tailored to particular cancers or pathogens.

The immune system can recognize and respond to differences between healthy/self and unhealthy/non-self-antigen, including antigens on cancerous cells. Acquired immunity has specific “memory” for these recognized antigens, and repeated exposure to the same antigen increases the response increases the level of induced protection against these previously-encountered cancers or pathogens.

Acquired immunity is mediated by the specialized immune cells, B and T lymphocytes. B lymphocytes produce and mediate their functions through the actions of antibodies. B lymphocyte dependent immune responses are referred to as “humoral immunity” because antibodies are detected in body fluids. T lymphocyte dependent immune responses are referred to as “cell mediated immunity” because effector activities are mediated directly by the local actions of effector T lymphocytes. The local actions of effector T lymphocytes are amplified through synergistic interactions between T lymphocytes and secondary effector cells, such as activated macrophages.

In order for immune cells to respond to foreign antigens, two signals must be provided by antigen-presenting cells (APCs) to resting T lymphocytes. The first signal, which confers specificity to the immune response, is transduced via the T cell receptor (TCR) when the TCR engages a specific peptide presented in the context of the major histocompatibility complex (MHC) (Rossy et al., Frontiers in Immunol. 3:1-12, 2012). The second signal, or co-stimulatory signal, is an antigen-independent co-signal that directs T cell function and T cell fate (Lenschow et al. (1996) Annu. Rev. Immunol. 14:233) (Chen and Flies Nat Rev Imm 2013). A co-stimulatory signal can be provided by temporary binding to one or more distinct cell surface polypeptides expressed by APCs (Jenkins, M. K. et al. (1988) J. Immunol. 140:3324-3330).

Using genetic engineering, significant progress has been made in activating and directing cells of the immune system to kill cancer cells and infected cells. For example, T cells have been genetically engineered to express molecules having extracellular components that bind particular target antigens and intracellular components that direct actions of the T cell when the extracellular component has bound the target antigen. As an example, the extracellular component can be designed to bind target antigens found on cancer cells or infected cells and, when bound, the intracellular component activates the T cell to destroy the bound cell. While this approach has provided anti-cancer effects, and in some situations, successful treatments, it is not always successful. One reason is that cancer cells can express proteins that result in inhibition of the immune system response. Thus, overcoming the inhibition of immune cells by cancer cells and maintaining an activated status of the immune system continue to be significant unmet needs.

### SUMMARY OF THE DISCLOSURE

The current disclosure provides recombinant proteins that stimulate an immune response in the presence of naturally inhibitory ligand binding. In certain examples, this approach can turn a signal that would inhibit the immune system response into a signal that stimulates the immune system. This approach is particularly useful to overcome inhibitory signals generated by cancer cells.

The recombinant proteins include an extracellular domain of an inhibitory immune cell protein and an intracellular domain of a stimulatory immune cell protein connected via a transmembrane domain. The present disclosure particularly describes the use of novel intracellular domains of stimulatory immune cell proteins including CD2, CD226, CRTAM, HAVCR1, SLAMF3, SLAMF5, SLAMF7, DR3, CD30, HVEM, and LIGHT in recombinant proteins. As indicated, the recombinant proteins can be used to stimulate immune cell activation in the fight against cancers and infectious diseases, among other uses.

Inhibitory immune cell proteins that can be used to create the extracellular domain of recombinant proteins described herein include Fas, CD200R, or SIRPα.

The extracellular domain can be connected to the intracellular domain with a transmembrane domain. A transmembrane domain can be derived from any membrane-bound or transmembrane protein. In particular embodiments, the transmembrane domain includes a transmembrane portion of CD28, Fas, CD30, DR3, or HVEM.

The present disclosure also particularly describes the novel and specific combinations of huFas with CD27 (e.g., huFas-huFas tm-CD27); huFas with CD40 (e.g., huFas-huFas tm-CD40); huFas with GITR (e.g., huFas-huFas tm-GITR); huFas with OX40 (e.g, huFas-huFas tm-OX40); muFas with CD27 (e.g., muFas-CD27tm-CD27); muFas with CD40 (e.g., muFas-CD40tm-CD40); muFas with GITR (e.g., muFas-GITRtm-GITR); muFas with OX40 (e.g., muFas-OX40tm-OX40); huCD200R with ICOS (e.g., huCD200R-12aas-CD28cys-ICOS); huCD200R with SLAMF1 (e.g., huCD200R-12aas-CD28cys-SLAMF1); muCD200R with ICOS (e.g., muCD200R-9aas ec-CD28cys-ICOS); muCD200R with SLAMF1 (e.g, muCD200R-9aas ec-CD28cys-SLAMF1); huSIRPα with ICOS (e.g, huSIRPα-12aas-CD28cys-ICOS); and huSIRPα with SLAMF1 (e.g., huSIRPα-12aas-CD28cys-SLAMF1).

Numerous recombinant proteins can be generated in various combinations and expressed in cells to tailor activation signals triggered in the presence of a particular ligand.

Immune system activation stimulation based on expression of the recombinant proteins disclosed herein can be further refined using, for example, multimerization and/or inducible expression.

The current disclosure also provides selecting particular recombinant proteins disclosed herein, and particular combinations of recombinant proteins disclosed herein, based on distinct attributes of these proteins and the clinical needs of patients. For example, as disclosed herein Fas-CD40 is particularly useful to stimulate cytokine production; Fas-OX40 is particularly useful to drive T cell expansion; Fas-CD27 is particularly useful to drive T cell proliferation (also under hypoxic conditions), while Fas-HVEM and SIRPα-ICOS are particularly effective at tumor cell lysis. Thus, to achieve optimal therapeutic efficacy for patients, a synergistic combination of Fas-CD40, Fas-CD27, and Fas-HVEM may be selected.

### BRIEF DESCRIPTION OF THE FIGURES

Some of the drawings submitted herein may be better understood in color. Applicant considers the color versions of the drawings as part of the original submission and reserves the right to present color images of the drawings in later proceedings.

FIG. 1. Schematic of a recombinant protein (which also can be referred to as a switch receptor or transform switch receptor (TSR) herein) wherein the recombinant protein has an inhibitory ectodomain connected to a costimulatory domain via a transmembrane domain.

FIGS. 2A, 2B. Expression of Fas ectodomain in murine CD8 T cells. Splenocytes from TCRgag mice were activated and transduced with a retroviral vector, including the indicated transgenes (GFP reporter). (2A) Representative flow cytometry plots showing the transduction efficiency in TCRgag CD8 T cells. (2B) Histograms represent the expression of the Fas ectodomain. Cells were gated on live cells followed by subsequent gating on Thy1.1+CD8+ and GFP positive cells. Numbers refer to mean fluorescence intensity (MFI) of Fas.

FIG. 3. Fas recombinant protein-expressing T cells show improved in vitro expansion. Splenocytes from TCRgag mice were activated and transduced with a retroviral vector, including the indicated transgenes. 7 days later, 1×10⁶ transgenic T cells (RO) were restimulated with 5×10⁶ irradiated APC, 3×10⁶ irradiated FBL leukemia, and rh-IL-2 (50 U/ml). Bar graph shows fold expansion of in vitro-derived T cells 5 days after stimulation with antigen. For each construct, mean with SD shown as bar graph with error bar. Each dot point represents an independent experiment. N=3. A one-way analysis of variance (ANOVA) method with multiple comparison was used to compare between groups (** p<0.01).

FIGS. 4A, 4B. Phenotypic characterization of Fas recombinant protein-expressing T cells. Splenocytes from TCRgag mice were activated and transduced with a retroviral vector, including the indicated transgenes. 7 days later, 1×10⁶ transgenic T cells were restimulated with 5×10⁶ irradiated APC, 3×10⁶ irradiated FBL leukemia, and rh-IL-2 (50 U/ml). 5 days after in vitro expansion with antigen, T cells were stained with surface markers in FACS buffer. Samples were then washed and immediately analyzed on a BD LSRFortessa flow cytometer. For the intracellular staining of GzmB, cells were fixed in BD Cytofix/Cytoperm fixation/permeabilization kit as per the manufacturer's protocol. Cells were washed with permeabilization buffer and stained for GzmB at 4 C for 45 min. (4A) Histograms show the expression of the activation markers (CD25, CD44, CD43 and GzmB) and exhaustion makers (PD-1, Tim-3, Lag-3 and TIGIT) on gated CD8+ GFP+. Numbers indicate the geometric mean fluorescence intensity (gMFI). gMFI of indicated markers were converted into heatmap (4B). The scale is the Z-score of mean.

FIGS. 5A-5C. Functional characterization of Fas recombinant proteins. Five days after in vitro expansion of TCR gag Fas with antigen, T cells were analyzed for intracellular cytokine production. T cells were stimulated with FBL leukemia (1:3) for 5 h in the presence of GolgiPlug. Cells were then stained for the expression of the surface markers (CD8, Thy1.1). For the intracellular staining of cytokines, cells were fixed in BD Cytofix/Cytoperm fixation/permeabilization kit as per the manufacturer's protocol. Cells were washed with permeabilization buffer and stained for INF-γ, IL-2 and TNF-α at 4C for 45 min. (5A) Representative flow cytometry plots show the production of cytokines. (5B) Bar graph depict the % production of INF-γ, IL-2 and TNF-γ. (5C) gMFI of indicated markers including the cytokines were converted into heatmap. The scale is the Z-score of mean.

FIGS. 6A, 6B. Fas TSR-T cells exhibit improved ability to lyse tumor cells and resist exhaustion following repeated stimulation in vitro. Five days after in vitro expansion of TCR gag Fas with antigen, T cells were co-cultured with FBL leukemia (1:4). The IncuCyte imaging system was used to measure, in real-time, the killing potential of the different TCRgag Fas T cells against FBL leukemia. To induce T cell exhaustion, additional target cells were added to the plate at the indicated times (arrows). ****P≤0.0001, ns=not significant.

FIG. 7. TCR Fas maintain their proliferative potential in a hypoxic setting. TCR transgenic P14 splenocytes were activated and transduced with a retroviral vector, including the indicated transgenes. T cells were labeled with CTV (Cell Trace Violet (5 μM)) for 20 min at 37° C. and washed 3× with complete media. CTV-labeled CD8 T cells were cultured in a pre-coated well with anti-CD3/CD28. Cells were incubated under hypoxia (1.5% O₂, 5% CO₂). Control CD8+ T cells were cultured under normoxia at 20% O₂ (5% CO₂).

FIGS. 8A-8C. Fas TSR-T cells show improved in vivo anti-tumor activity. To determine the ability of Fas TSR-T cells to control tumor growth in vivo, 5×10⁶ FBL leukemia were injected into mice (I.P.) (8A) 5 days after the tumor inoculation, mice received 1e6 Fas TSR-T cells at least 6 hour after lymphodepletion preconditioning. (8B) Bar graph depicts the frequency of transferred Thy1.1 Fas TSR-T cells at day 7 post-transfer. (8C) Kaplan-Meier survival curve of mice.

FIG. 9. Summary of the phenotypic and functional characteristics of Fas TSR-T cells demonstrating that different intracellular domains provide different benefits in terms of proliferation, expansion, activation, cytokine production, effector-like function, serial killing and exhaustion.

FIGS. 10A, 10B. CD200R recombinant protein expression in primary murine T cells. C57BL/6 splenocytes were transduced with a retroviral vector including the indicated transgene. T cells were stained with specific antibody to CD200R and analyzed by flow cytometry. GFP indicates a vector control; Tr indicates truncated CD200R (tailless). CD200R recombinant protein-expressing T cells exhibit surface expression in primary murine T cells.

FIGS. 11A, 11B. Enrichment of transduced murine T cells in a mixed population including nontransduced T cells after 3 weekly cycles of stimulation with irradiated tumor cells and splenocytes. C57BL/6 splenocytes were transduced with a retroviral vector including the indicated transgene with a GFP expression gene linked by P2A. T cells were analyzed by flow cytometry. GFP indicates a vector control. Effective recombinant protein-expressing T cells outcompete nontransduced T cells and accumulate with multiple stimulations.

FIG. 12A, 12B. Splenocytes from P14 mice were activated and transduced with a retroviral vector, including the indicated transgenes. Histograms represent the expression of the SIRPα ectodomain. Cells were gated on live cells followed by subsequent gating on Thy1.1+CD8+ cells.

FIGS. 13A, 13B. SIRPα TSR-T cells exhibit enhanced ability to lyse tumors. Engineered T cells were co-cultured with NIR-labeled KPC pancreatic tumor cells at a 1:1 ratio. The IncuCyte imaging system was used to measure, in real-time, the killing potential of the different SIRPα TSR-T cells against pancreatic tumor. Tumor cells were quantified at the end of study (6 days) (total NIR area).

FIGS. 14A-14E. Sequences supporting the disclosure. (14A) Extracellular domain sequences with and without junction amino acids. (14B) Transmembrane domain sequences. (14C) Intracellular domain sequences. (14D) Other sequences. (14E) Recombinant protein sequences.

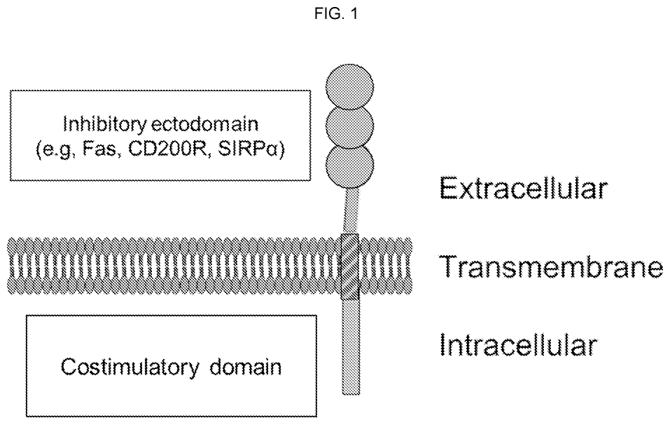

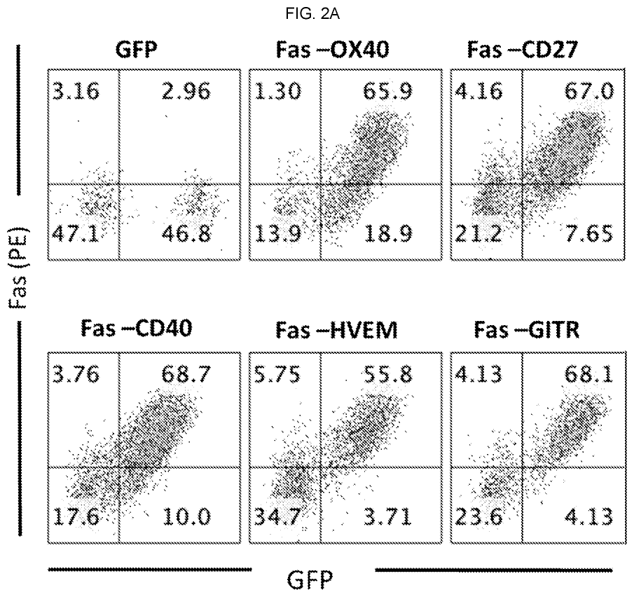

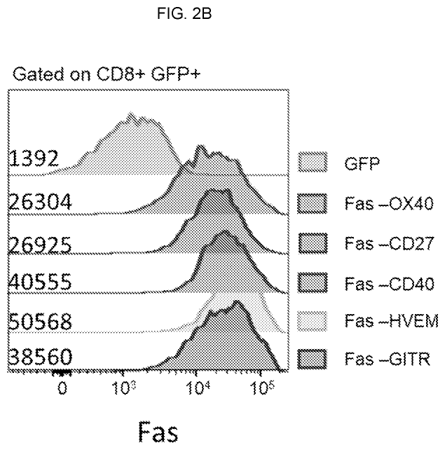

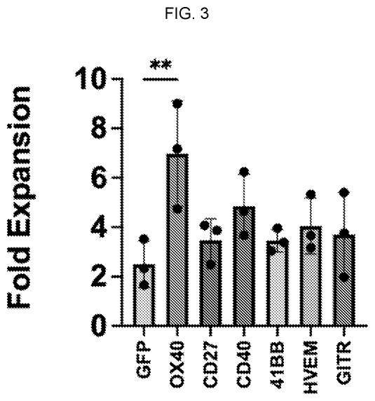

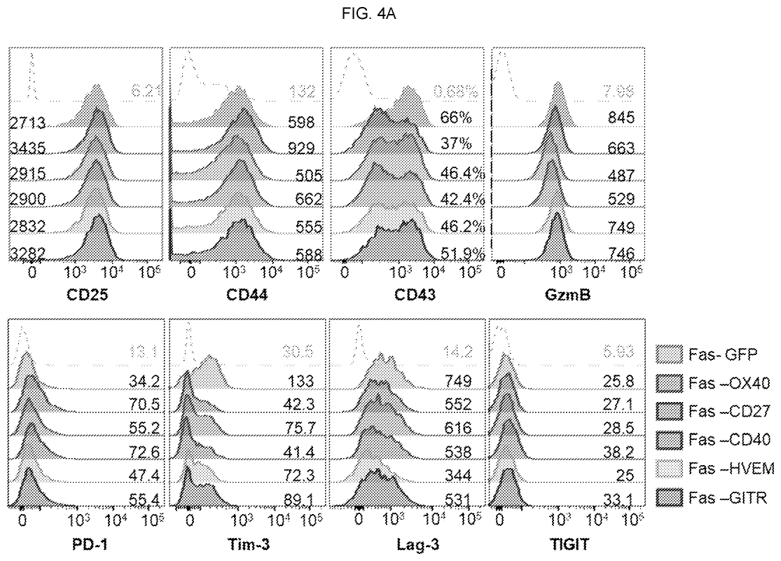

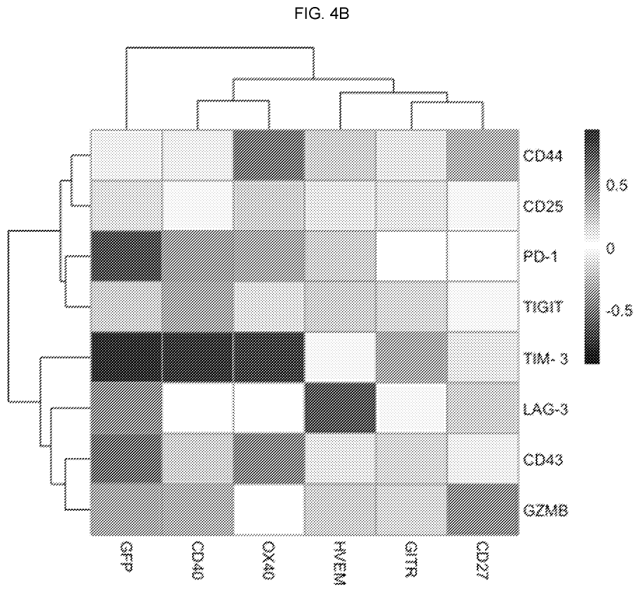

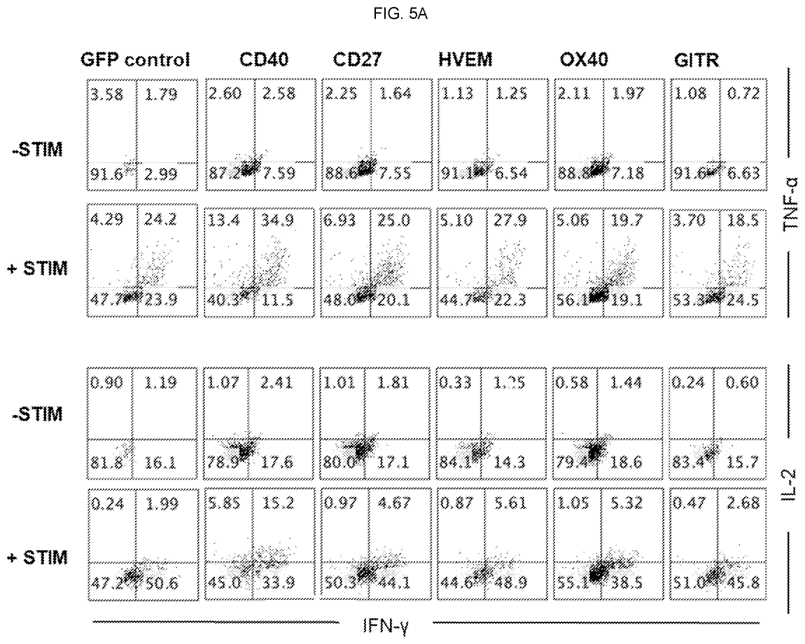

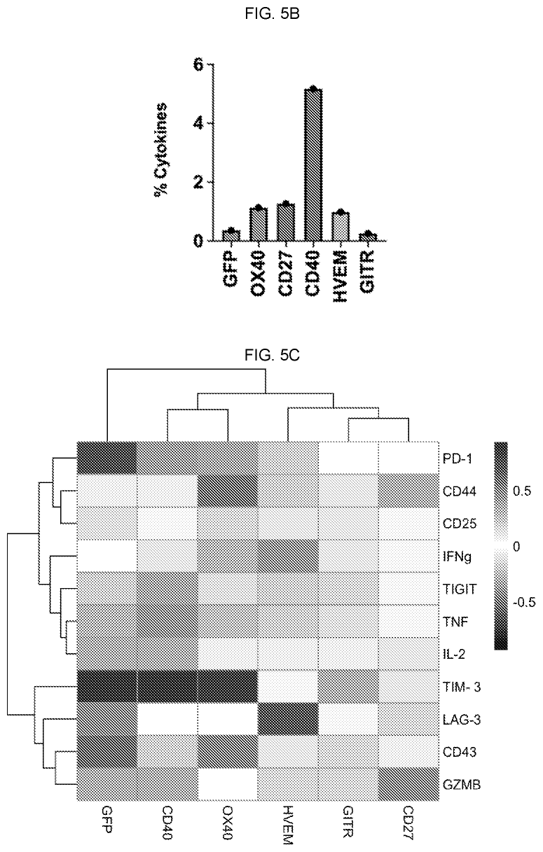

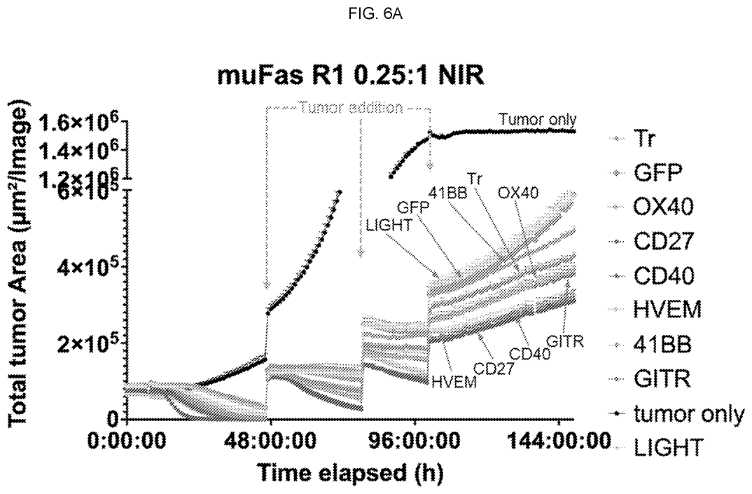

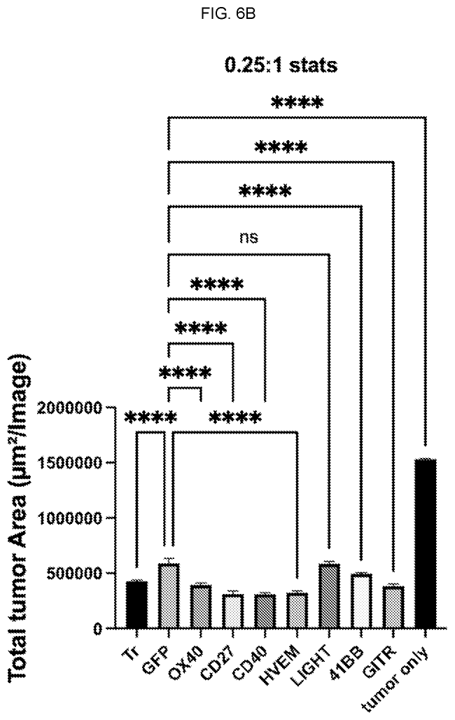

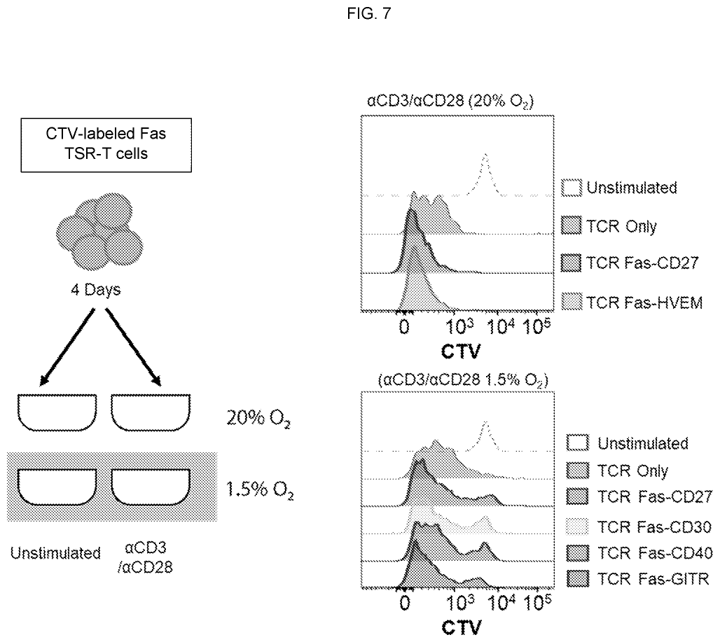

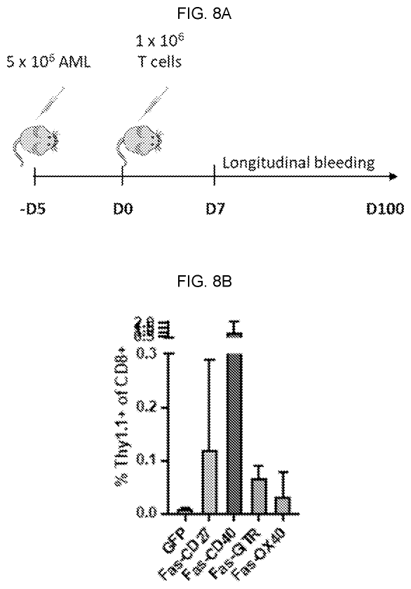

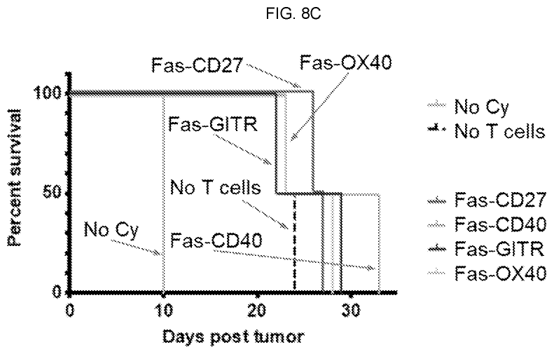

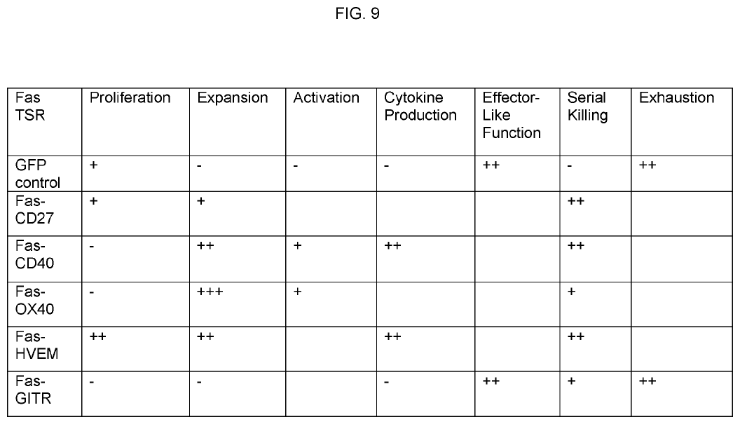

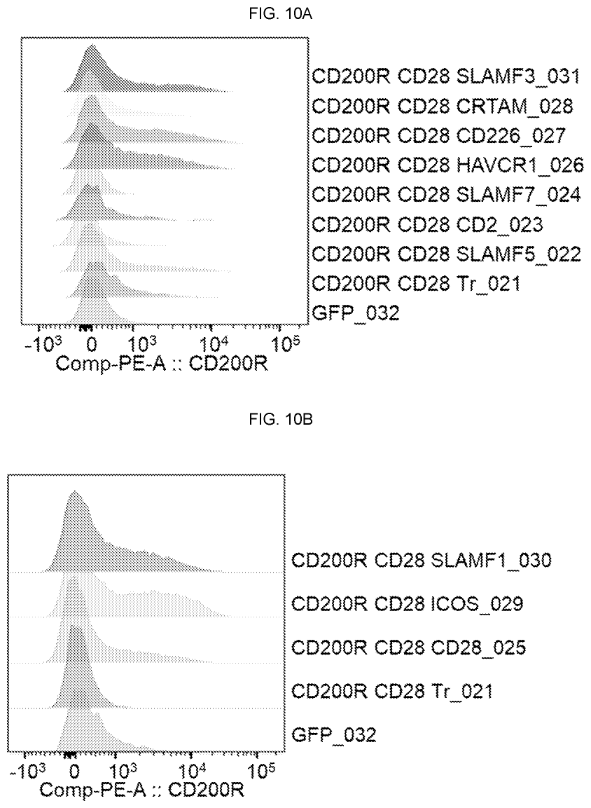

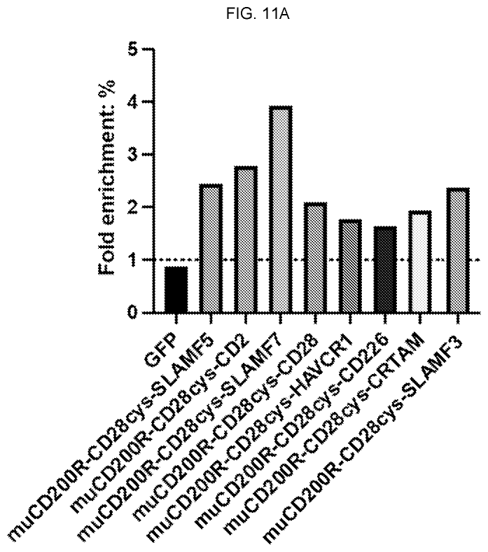

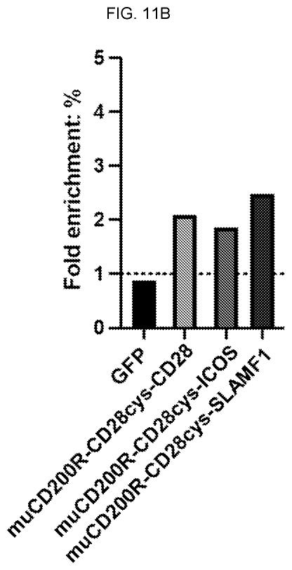

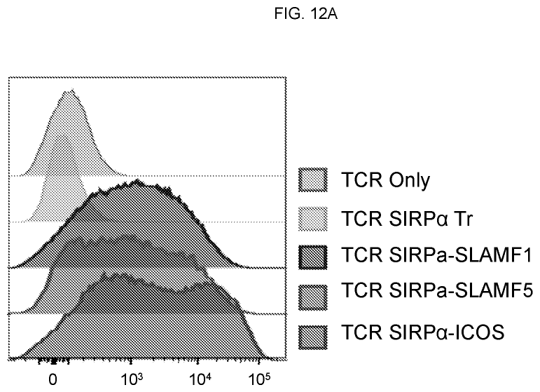

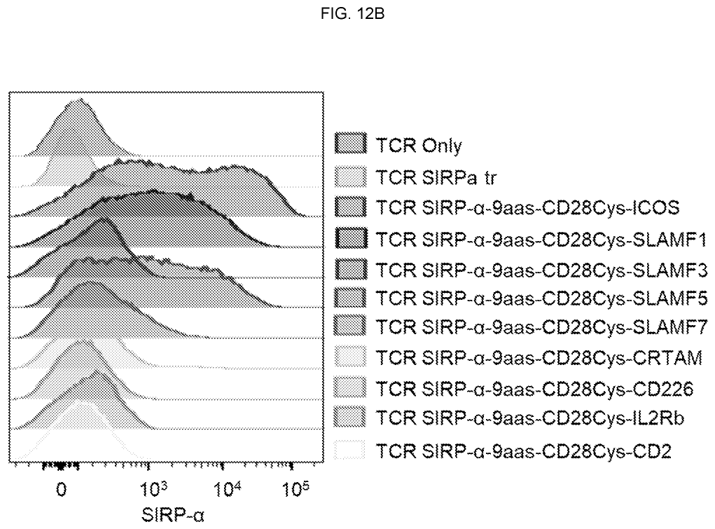

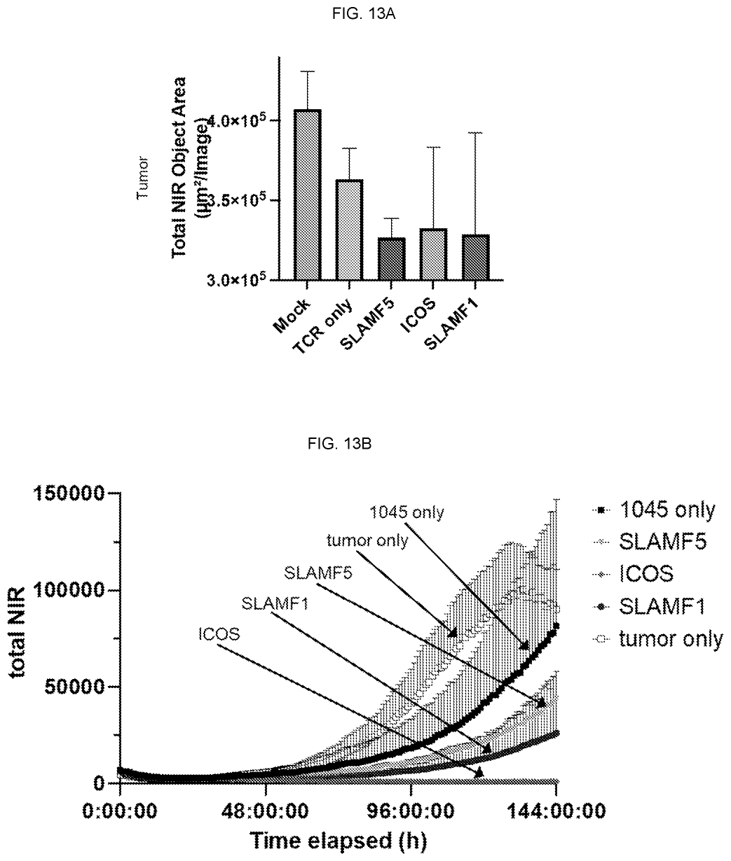

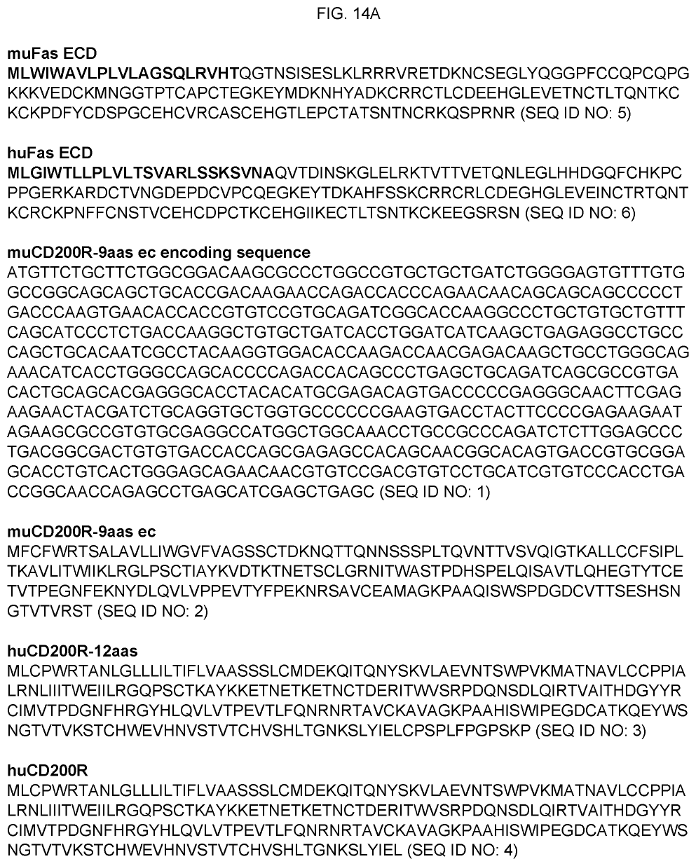

### DETAILED DESCRIPTION

Using genetic engineering, significant progress has been made in activating and directing cells of the immune system to kill cancer cells and infected cells. Clinical trials with chimeric antigen receptor (CAR)-expressing T cells (CAR-T), for example, have shown positive responses and cancer reduction in some patients. However, in others they have failed to provide prolonged anti-cancer activity. One reason is that cancer cells can express proteins that result in inhibition of the immune system response. Thus, there is a need to overcome the inhibition of immune cells by cancer cells and a need to maintain the immune system in an activated status.

The current disclosure provides recombinant proteins that stimulate an immune response in the presence of naturally inhibitory ligand binding. In certain examples, this approach can turn a signal that would inhibit the immune system response into a signal that stimulates the immune system. This approach has many uses when enhancement of the immune system response is desired, and is particularly useful to overcome inhibitory signals generated by cancer cells.

The current disclosure provides recombinant proteins that stimulate an immune response in the presence of naturally inhibitory ligand binding. The recombinant proteins include an extracellular domain of an inhibitory immune cell protein and an intracellular domain of a stimulatory immune cell protein connected via a transmembrane domain. The recombinant proteins can be used to stimulate immune cell activation in the fight against cancers and infectious diseases, among other uses. This disclosure provides new costimulatory domains within a recombinant proteins including CD2, CD226, CRTAM, HAVCR1, SLAMF3, SLAMF5, SLAMF7, DR3, CD30, HVEM, and LIGHT. Recombinant proteins disclosed herein are beneficial because of their ability to convert naturally inhibitory signals into stimulatory signals. As such, recombinant proteins can be used in adoptive cell therapy of cancer, autoimmune disease, infectious disease, and graft rejection. Vectors described herein can be used for tumor-infiltrating lymphocyte (TIL) therapy or in situ genetic engineering of endogenous immune cells.

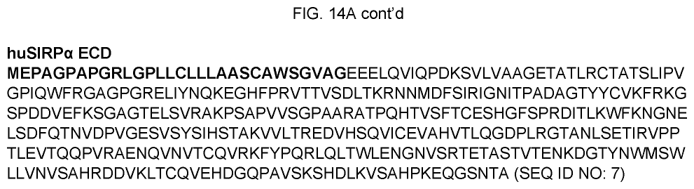

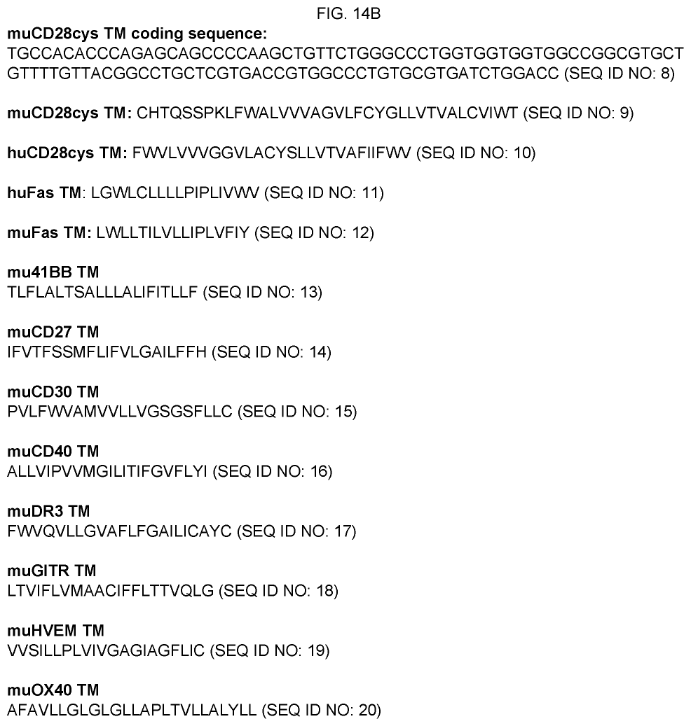

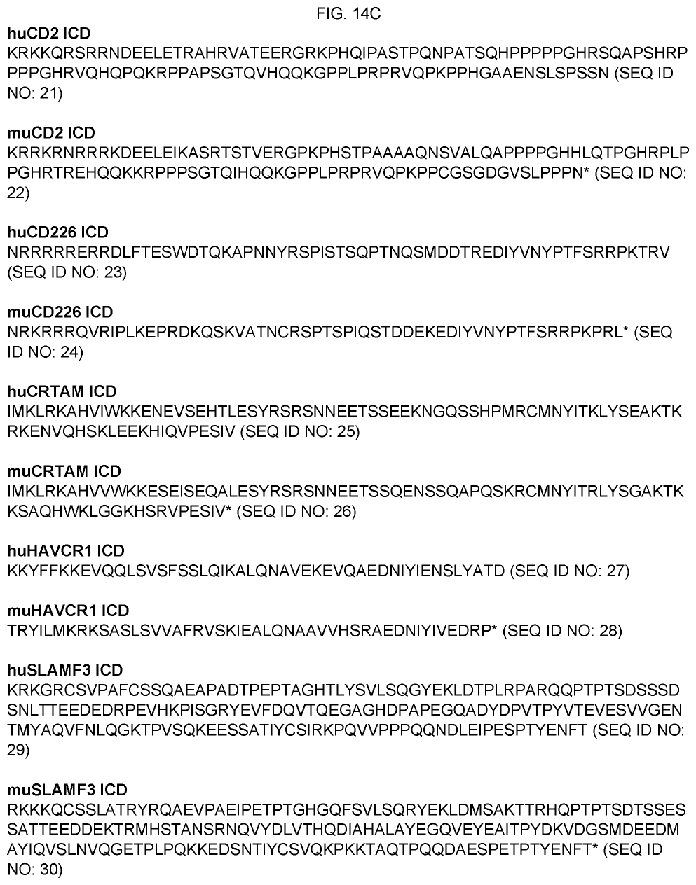

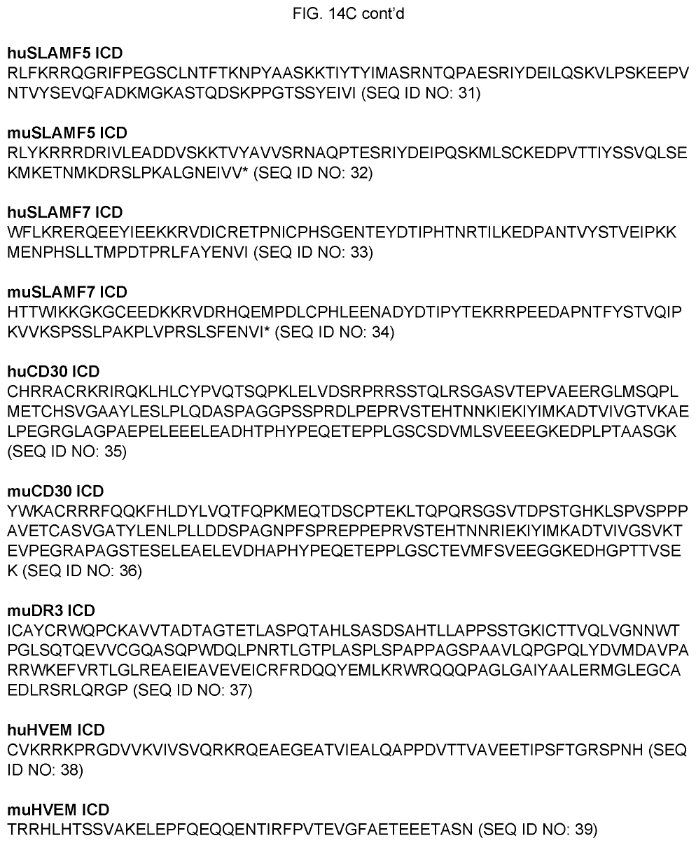

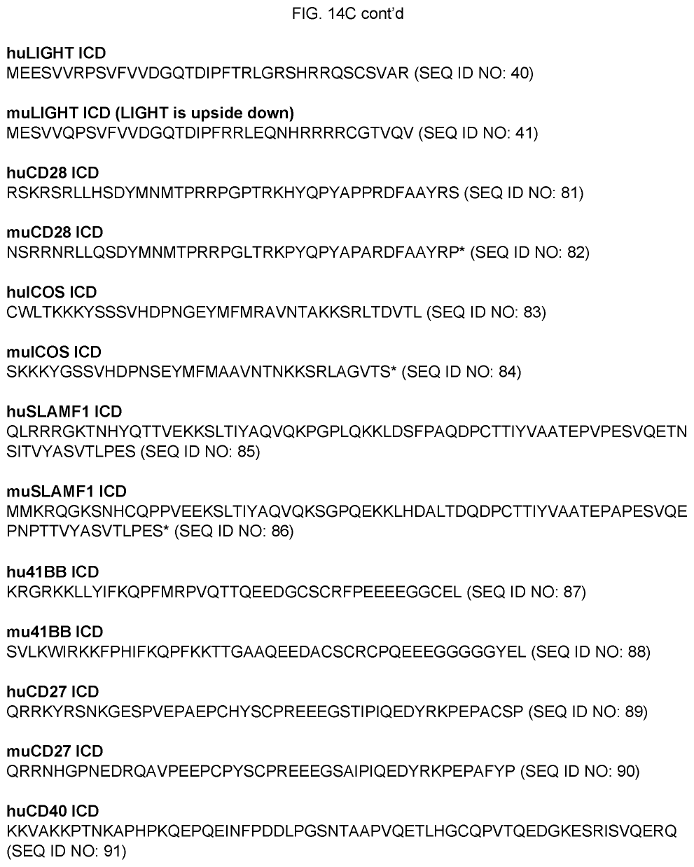

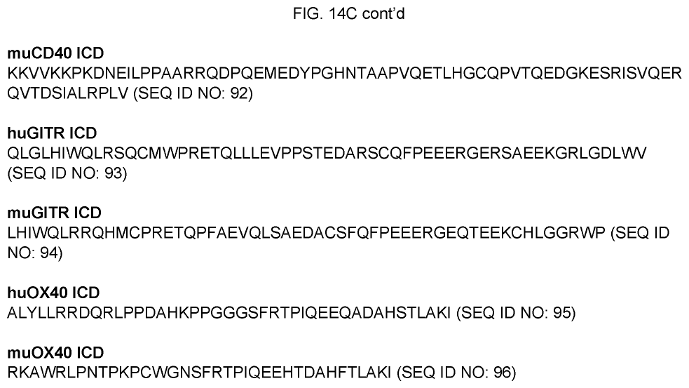

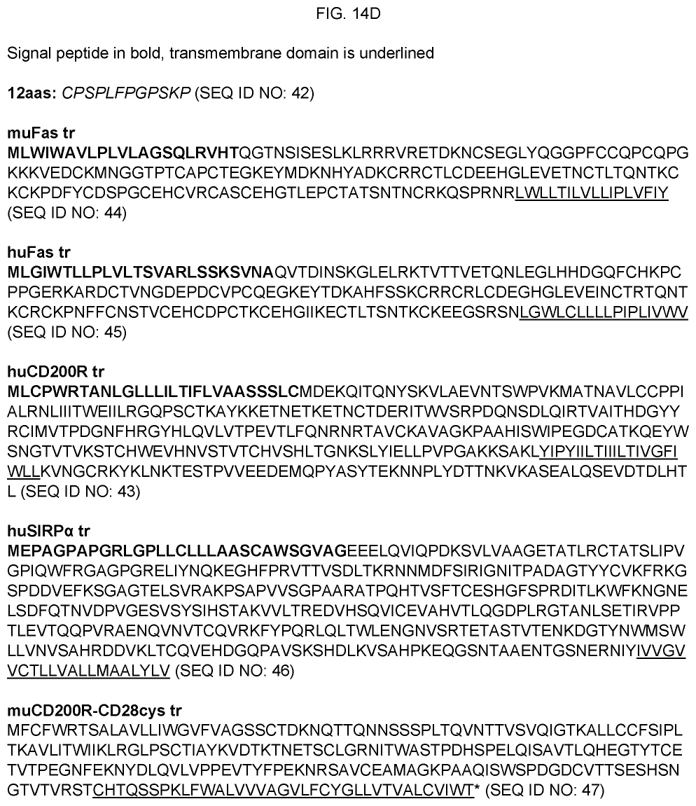

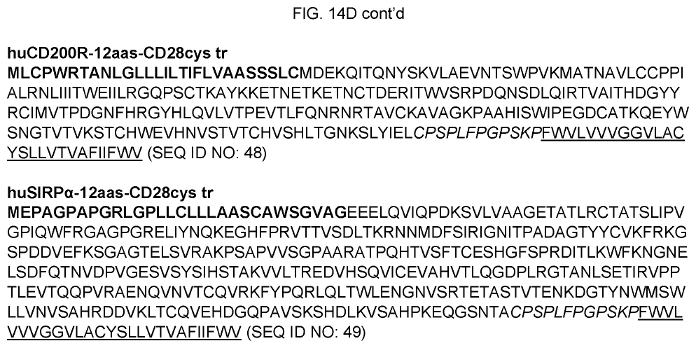

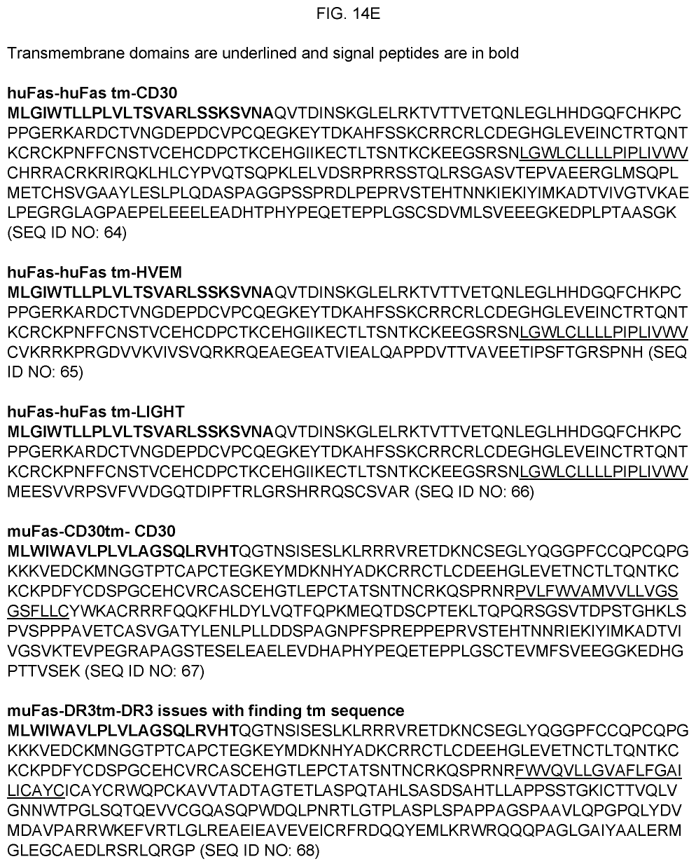

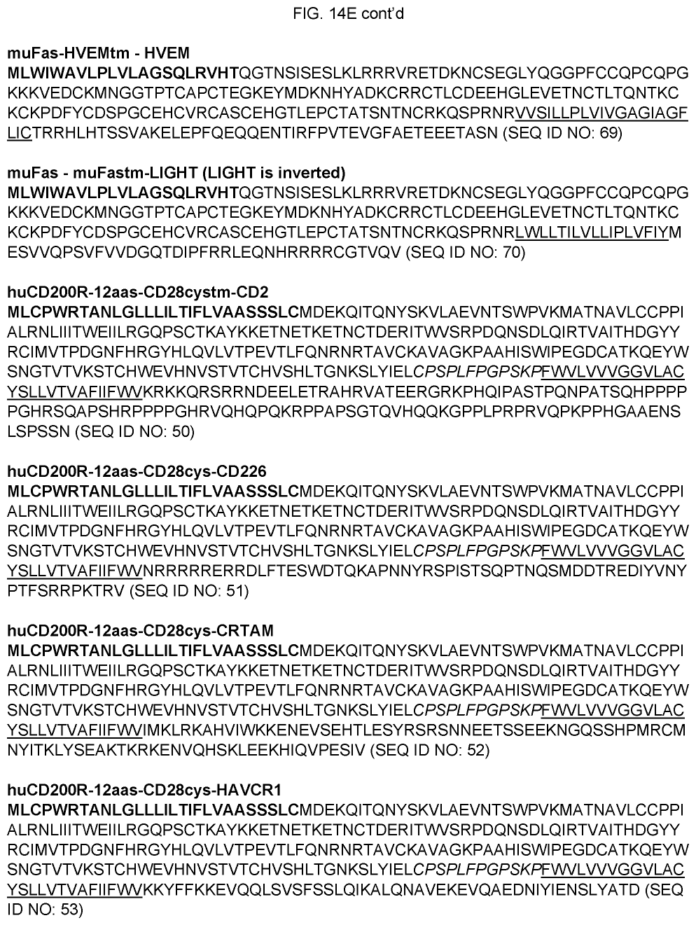

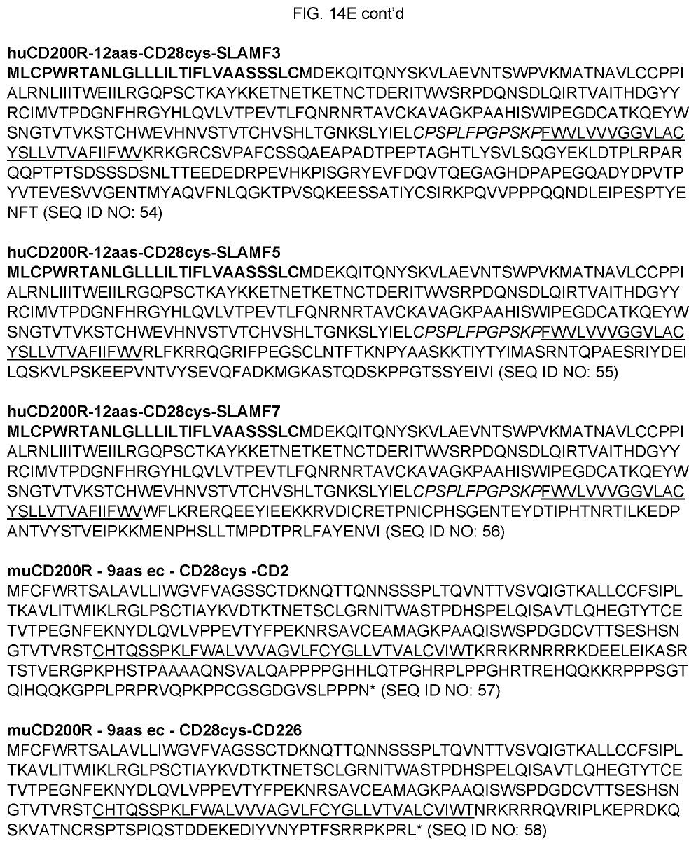

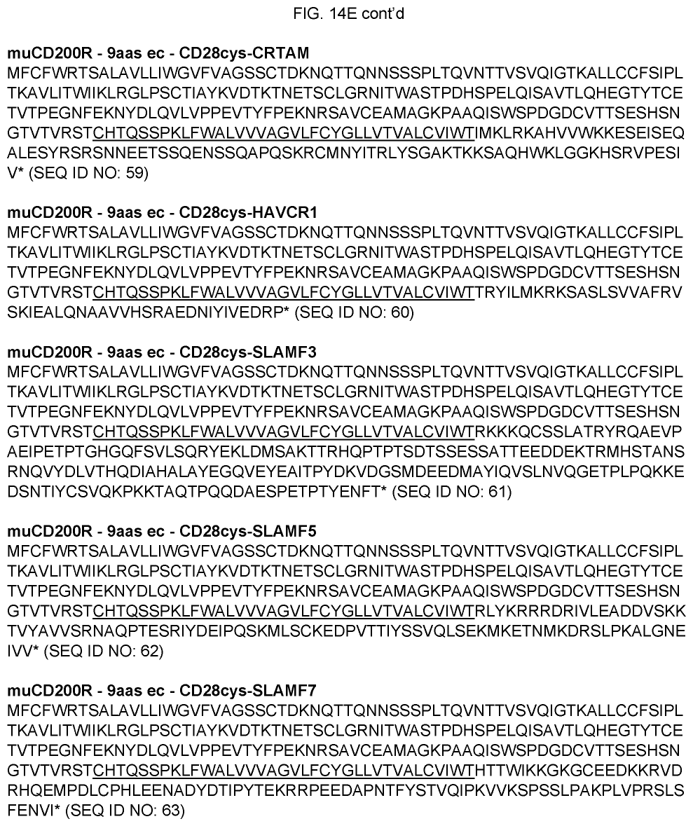

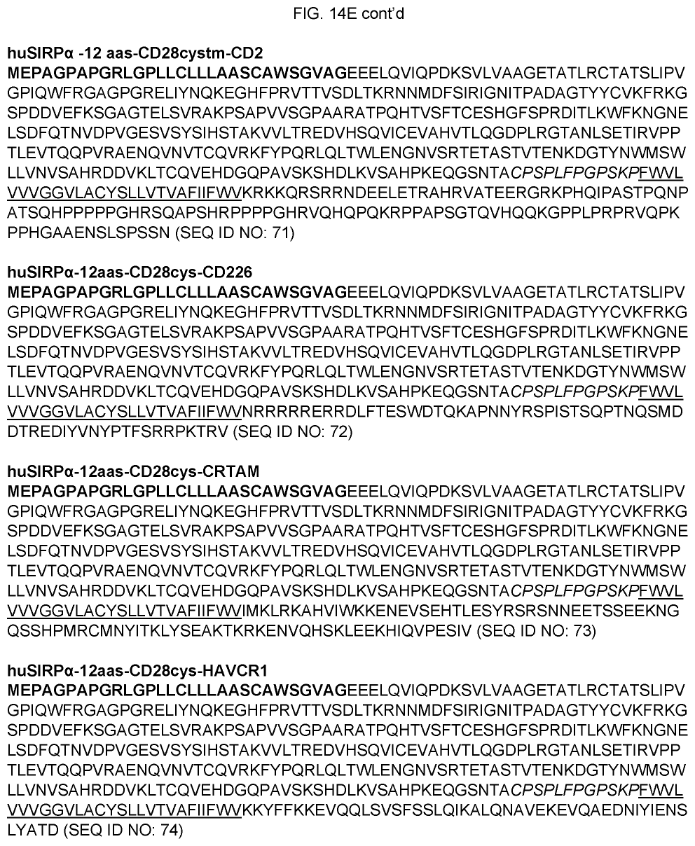

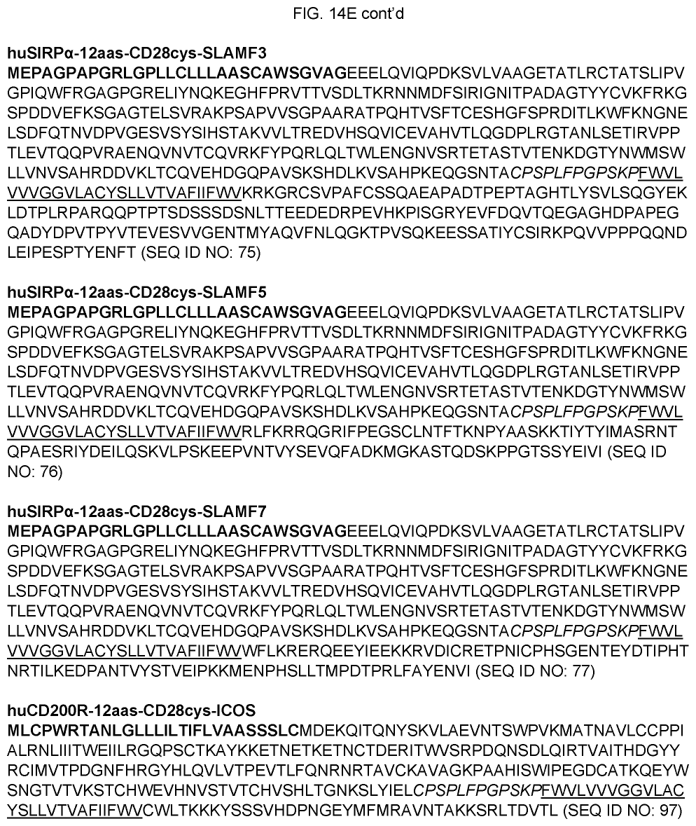

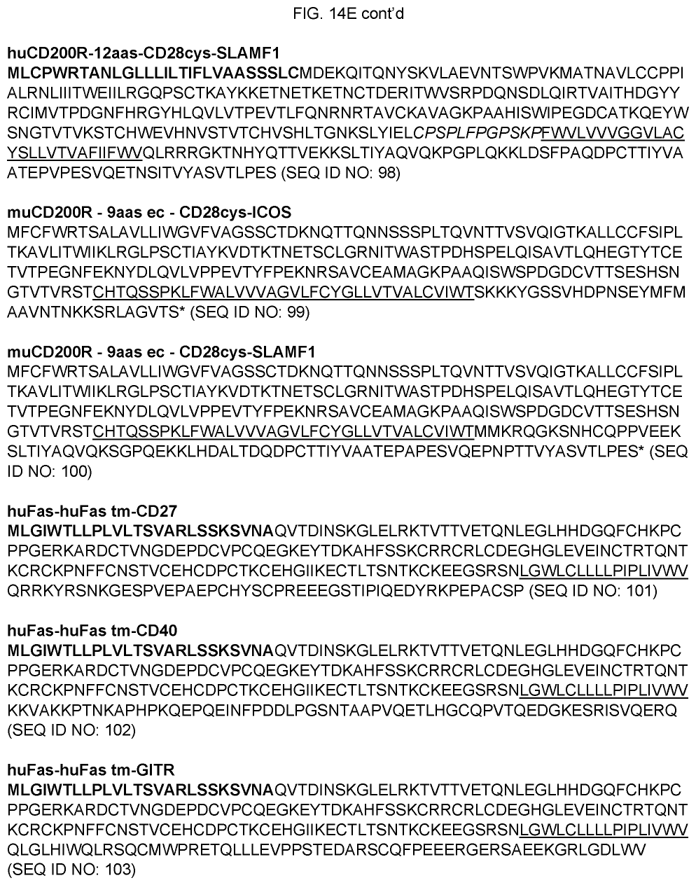

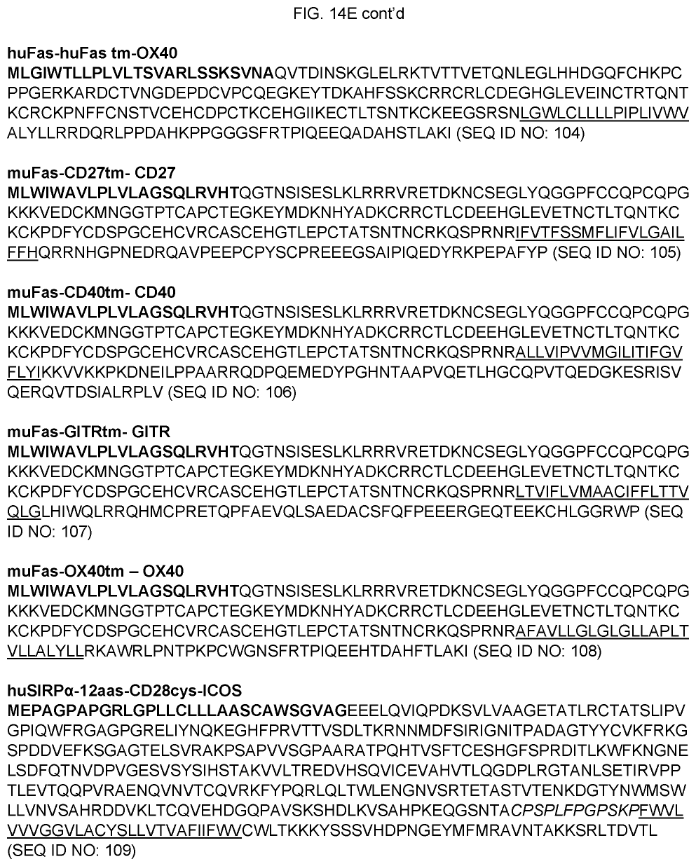

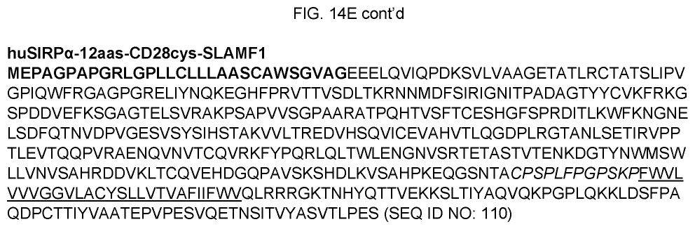

Inhibitory immune cell proteins that can be used to create recombinant proteins described herein include Fas, CD200R, and SIRPα. In particular embodiments, recombinant proteins include an extracellular domain including an extracellular portion of an inhibitory immune cell protein selected from huFas, muFas, huCD200R, muCD200R, and huSIRPα. In particular embodiments, extracellular portions of inhibitory immune cell proteins are described in FIG. 14A.

Fas or tumor necrosis factor receptor superfamily member 6 forms the death-inducing signaling complex (DISC) upon binding its ligand and is important in apoptosis. The interaction with its ligand leads to apoptosis of thymocytes that fail to rearrange their TCR genes correctly and of those that recognize self-antigens.

CD200 receptor (CD200R) is an immunoregulatory receptor important in the maintenance of immune tolerance. The activation of CD200R regulates the expression of pro-inflammatroy molecules such as tumor necrosis factor (TNFα), interferons, and inducible nitric oxide (iNOS).

Signal regulatory protein α (SIRPα) is a regulatory membrane glycoprotein that acts as an inhibitory receptor and interacts with the transmembrane protein CD47. This binding negatively controls effector function of innate immune cells.

Stimulatory immune cell proteins that can be used create recombinant proteins described herein include an intracellular domain including an intracellular portion of a stimulatory immune cell protein selected from CD2, CD226, CRTAM, HAVCR1, SLAMF3, SLAMF5, SLAMF7, DR3, CD30, HVEM, and LIGHT. In particular embodiments, intracellular portions of stimulatory immune cell proteins are described in FIG. 14C.

CD2 (cluster of differentiation 2) is a costimulatory receptor expressed on T and natural killer (NK) cells that binds to LFA3. CD2 is important in the formation and organization of the immunological synapse that is formed between T cells and antigen-presenting cells upon cell-cell conjugation and associated intracellular signaling.

CD226 is a member of the immunoglobulin superfamily and is a functional protein initially expressed on NK and T cells. CD226 is closely related to the occurrence of autoimmune diseases, infectious diseases, and tumors.

Cytotoxic and regulatory T cell molecule (CRTAM) plays a role in regulating CD8+ T cell retention and eventually effector function. Nectin-like molecule-2 is its ligand.

Hepatitis A virus cellular receptor 1 (HAVCR1), or T cell immunoglobin and mucin domain 1 (TIM-1), plays a critical role in regulating immune cell activity particularly regarding the host response to viral infection. It is also involved in allergic response, asthma, and transplant tolerance.

Signaling lymphocytic activation molecule 1 (SLAMF1), or CD150, is a member of the SLAM family along with SLAMF3, SLAMF5, and SLAMF7. SLAM receptors modulate the activation and differentiation of a variety of immune cells and are involved in the regulation and interconnection of the innate and adaptive immune response.

Death receptor 3 (DR3) is also referred to as TRAMP, LARD, WSL-1, and TNFRSF member 25 (TNRFSF25). DR3 is a death-domain-containing tumor necrosis factor family receptor expressed on T cells. Its ligand, TL1A (also referred to as TNFSF15 or VEGI), costimulates T cells to produce a wide variety of cytokines and can promote expansion of activated and regulatory T cells. DR3 costimulates T cell activation and is unique because it signals through an intracytoplasmic death domain and the adapter protein TRADD (Meylan, et al., 2011. Immunol Rev. 244(1): 10.1111).

CD30 is a TNFRSF member that is often expressed in hematopoietic malignancies such as large cell lymphoma and Hodgkin lymphoma. The CD30 ligand, also referred to as CD30L, TNFSF8, or CD153, is a membrane-bound cytokine. CD30 signaling controls T-cell survival, regulates peripheral T-cell responses, and downregulates cytolytic capacity (Wu, et al., Immune Biology of Allogeneic Hematopoietic Stem Cell Transplantation (Second Edition), 2019).

Herpesvirus entry mediator (HVEM) is the specific ligand for B-and T-lymphocyte attenuator (BTLA). BTLA is an immune-regulatory receptor that is expressed on B-and T-, and all mature lymphocytes. BTLA, also referred to as CD272, is in the CD38 family along with PD1 and CTLA-4 while HVEM belongs to the TNFR family. The interaction of HVEM and BTLA plays an important role in immune tolerance and immune response (Yu et al., 2019, Front. Immunol. ht tps://doi.org/10.3389/fimmu.2019.00617).

LIGHT (also known as tumor necrosis superfamily member 14, CD258, and HVEML) is a secreted protein of the TNF superfamily. Upon binding its ligand, herpesvirus entry mediator (HVEM), LIGHT interacts with two receptors, lyphotoxin-β receptor (LTBR) and herpesvirus entry mediator (HVEM) to enhance T cell proliferation and cytokine production.

The present disclosure particularly describes the novel and specific combinations of huFas with CD27 (e.g., huFas-huFas tm-CD27); huFas with CD40 (e.g., huFas-huFas tm-CD40); huFas with GITR (e.g., huFas-huFas tm-GITR); huFas with OX40 (e.g, huFas-huFas tm-OX40); muFas with CD27 (e.g., muFas-CD27tm-CD27); muFas with CD40 (e.g., muFas-CD40tm-CD40); muFas with GITR (e.g., muFas-GITRtm-GITR); muFas with OX40 (e.g., muFas-OX40tm-OX40); huCD200R with ICOS (e.g., huCD200R-12aas-CD28cys-ICOS); huCD200R with SLAMF1 (e.g., huCD200R-12aas-CD28cys-SLAMF1); muCD200R with ICOS (e.g., muCD200R-9aas ec-CD28cys-ICOS); muCD200R with SLAMF1 (e.g, muCD200R-9aas ec-CD28cys-SLAMF1); huSIRPαwith ICOS (e.g, huSIRPα-12aas-CD28cys-ICOS); and huSIRPα with SLAMF1 (e.g., huSIRPα-12aas-CD28cys-SLAMF1).

Inducible T cell costimulatory (ICOS), or CD278, is a CD28-superfamily costimulatory molecule expressed on activated T cells. It forms homodimers and plays a role in cell-cell signaling, immune responses, and regulation of cell proliferation.

CD27 is a TNFRSF member that is a transmembrane protein. It is expressed on the majority of CD4+ and CD8+ resting T cells. The ligand for CD27 is CD70 and their interaction enhances T cell activation with regards to proliferation. Improved signaling of CD27 is shown with hexamerization (Thieman et al., Front. Oncol. 8, 2018).

CD40, also referred to as TNFRSF member 5 (TNFRSF5), or CD40 ligand receptor is a costimulatory protein found on APCs and is required for activation. CD40 contains 277 amino acids of which 20 amino acids at the N terminus represent the signal sequence. A transmembrane domain is located at resides 194-215 and the cytoplasmic domain is located at residues 216-277. The nucleotide sequence of CD40 (1177 bp) is available in public databases (see Genbank accession no. NM—001250). CD40 and various isoforms are described by Tone et al. Proc. Natl. Acad. Sci. U.S.A. 98 (4), 1751-1756 (2001). CD40 is expressed by monocytes and B cells binds to CD40-L (a.k.a. CD40 ligand or CD153) expressed by activated T cells.

Glucocorticoid-induced TNFR-related protein (GITR) is a member of the TNFR superfamily which is expressed in various cells, including T cells, natural killer cells and some myeloid cells. GITR is activated by its ligand, GITRL, which upon engagement modulates both innate and adaptive immune responses.

OX40, also referred to as CD134, TNFRSF member 4 (TNFRSF4), ACT35 and TXGP1L, is a 50 kDa glycoprotein. The ligand for OX40, OX40L (also referred to as CD252), has been reported to be expressed on endothelial cells and activated APCs including macrophages, dendritic cells, B cells and natural killer cells. Binding between CD40 on APCs increases OX40L expression. Expression of OX40 on T cells can be induced following signaling via the T cell antigen receptor. For example, OX40 is expressed on recently activated T cells at the site of inflammation. CD4 and CD8 T cells can upregulate OX40 under inflammatory conditions. Costimulatory signals from OX40 promote T cell division, survival, and suppress the differentiation and activity of Treg T cells (Croft Immunol Rev 2009).

The extracellular domain can be connected to the intracellular domain with a transmembrane domain. The transmembrane domain can anchor the expressed molecule in a modified cell's membrane. Within recombinant proteins disclosed herein, transmembrane domains can be any transmembrane domain that has a three-dimensional structure that is thermodynamically stable in a cell membrane. Transmembrane domains generally range in length from 15 to 30 amino acids. The structure of a transmembrane domain can include an alpha helix, a beta barrel, a beta sheet, a beta helix, or any combination thereof.

The transmembrane domain may be derived either from a natural or from a recombinant source. When the source is natural, the domain may be derived from any membrane-bound or transmembrane protein. In particular embodiments, a transmembrane domain may include at least the transmembrane region(s) of: the a, B, or Z chain of the T-cell receptor; CD28; CD27; CD38; CD45; CD4; CD5; CD8; CD9; CD16; CD22; CD33; CD37; CD64; CD80; CD86; CD134; CD137; and/or CD154. In particular embodiments, a transmembrane domain may include at least the transmembrane region(s) of: KIRDS2; OX40; OX40L; CD2; LFA-1; ICOS; ICOSL; 4-1BB; 4-1BBL; GITR; GITRL; CD40; CD40L; CD30; CD30L; FLT3; FLT3L; Fyn; FynL; Lck; LckL; LAT; LATL; LRP; LRPL; LIGHT; DR3; DR3L; CD27; CD27L; CD25; CD28; CD80; CD86,; CD79a; CD79aL; CD79b; CD79bL; CD84 (SLAMF5); DAP10; DAP10L; DAP12; DAP12L; BAFFR; HVEM; SLAMF7; NKp80; NKp44; NKp30; NKp46; NOTCH1; NOTCH1L; NOTCH2; NOTCH2L; NOTCH3; NOTCH3L; NOTCH4; NOTCH4L; ROR2; ROR2L; Ryk; RykL; Slp76; Slp76L; pTa; pTaL; TCRα; TCRB; HAVCR1 (TIM1); TIM1L; TRIM; TRIML; Zap70; Zap70L; PTCH2; PTCH2L; CD122; CD132; CD226; CD160; CD19; CARD11; CARD11L; CRTAM (CD355); IL2Rß; IL2Ry; IL7Ra; ITGA1; VLA1; CD49a; ITGA4; IA4; CD49D; ITGA6; VLA-6; CD49f; ITGAD; CDI Id; ITGAE; CD103; ITGAL; CDI Ia; ITGAM; CDI Ib; ITGAX; CDI Ic; ITGB1; CD29; ITGB2; CD18; ITGB7; TNFR2; DNAM1; SLAMF1 (CD150 or SLAM); SLAMF1L; SLAMF4; CD84; CD96; CEACAM1; CRT AM; Ly9 (SLAMF3); PSGL1; CD100; SLAMF6 (NTB-A, Lyl08); BLAME (SLAMF8); SELPLG; LTBR; PAG/Cbp; NKG2D; NKG2DL; NKG2C; FcRα; FcRαL; FcRβ; FcRβL; FcRγ; FcRγL; CD3ε; CD3δ; or CD3ζ. In particular embodiments, the transmembrane domain includes a transmembrane portion of CD28, Fas, CD30, DR3, and HVEM. In particular embodiments, a transmembrane domain sequences are described in FIG. 14B.

In particular embodiments, the transmembrane domain can extend into the extracellular space. In particular embodiments, the transmembrane domain extends into the extracellular space 15 amino acids, 14 amino acids, 13 amino acids, 12 amino acids, 11 amino acids, 10 amino acids, 9 amino acids, 8 amino acids, 7 amino acids, 6 amino acids, 5 amino acids, 4 amino acids, 3 amino acids, 2 amino acids, or 1 amino acid. In particular embodiments, CD28cys extends into the extracellular space 9 amino acids. In particular embodiments, the extracellular domain can be truncated to accommodate the transmembrane domain extending into the extracellular space. In particular embodiments, muCD200R-9aas is truncated by 9 amino acids on the end closest to the transmembrane domain. In particular embodiments, huCD200R-12aas is truncated by 12 amino acids on the end closest to the transmembrane domain.

In particular embodiments, the transmembrane domain can include predominantly hydrophobic residues such as leucine and valine. In particular embodiments, the transmembrane domain can include a triplet of phenylalanine, tryptophan and valine found at each end of the transmembrane domain.

In preferred embodiments, the recombinant proteins disclosed herein utilize only segments of naturally occurring proteins to minimize any potential immune response when expressed in vivo. While these embodiments are preferred, in some instances junction amino acids may be present between segments of the recombinant proteins. Junction amino acids refer to short amino acid sequences, for example, 20 amino acids or less. Exemplary glycine-serine junction amino acids include GGGGSGGGGS (SEQ ID NO: 78), GGSGGSGGS (SEQ ID NO: 79), and GGGGS (SEQ ID NO: 80). In particular embodiments, a glycine-serine (GS) doublet can be used as a suitable junction amino acid linker. In particular embodiments, a single amino acid, e.g., an alanine, a glycine, can be used as a suitable junction amino acid.

In certain instances, recombinant proteins described herein include components of a Type I protein and a Type II protein. Type I proteins include their N-terminus extracellularly when expressed while Type II proteins include their N-terminus intracellularly when expressed. Generally, costimulatory immune cell proteins (e.g., OX40) are Type I proteins while costimulatory protein ligands (e.g., OX40L) are Type II proteins.

To fuse a Type I protein with a Type II protein, intracellular and/or transmembrane regions may be inverted from their native configuration (e.g., so that the intracellular tail terminus becomes the membrane proximal portion of the intracellular region). In additional embodiments, the N-terminal to C-terminal position of a stimulatory protein intracellular signaling domain is brought towards the N-terminal position. For example, from N-terminal to C-terminal expression, costimulatory immune cell proteins generally proceed from extracellular signaling domain to transmembrane domain to intracellular signaling domain. N-terminal to C-terminal expression of costimulatory immune cell protein ligands generally proceed from intracellular signaling domain to transmembrane domain to extracellular signaling domain. Certain recombinant proteins described herein include from N-terminal to C-terminal expression, an extracellular signaling domain of an inhibitory immune cell signaling domain to a transmembrane domain to an inverted intracellular signaling domain of a costimulatory immune cell ligand.

Numerous recombinant proteins can be generated in various combinations and expressed in cells to tailor activation signals triggered in the presence of a particular ligand.

Immune system activation stimulation based on expression of the recombinant proteins disclosed herein can be further refined using, for example, chemically-induced multimerization systems (CIMS) and/or inducible expression.

In particular embodiments, a recombinant protein includes an extracellular domain selected from the extracellular portion of huFas, muFas, huCD200R, muCD200R, or huSIRPα; a transmembrane domain; and an intracellular domain selected from the intracellular portion of CD2, CD226, CRTAM, HAVCR1, SLAMF3, SLAMF5, SLAMF7, DR3, CD30, HVEM, or LIGHT.

In particular embodiments, a recombinant protein includes an extracellular domain including an extracellular portion of huFas, a transmembrane domain including the transmembrane portion of huFas, and an intracellular domain including an intracellular portion of CD30.

In particular embodiments, a recombinant protein includes an extracellular domain including an extracellular portion of huFas, a transmembrane domain including the transmembrane portion of huFas, and an intracellular domain including an intracellular portion of HVEM.

In particular embodiments, a recombinant protein includes an extracellular domain including an extracellular portion of huFas, a transmembrane domain including the transmembrane portion of huFas, and an intracellular domain including an intracellular portion of LIGHT.

In particular embodiments, a recombinant protein includes an extracellular domain including an extracellular portion of muFas, a transmembrane domain including the transmembrane portion of CD30, and an intracellular domain including an intracellular portion of CD30.

In particular embodiments, a recombinant protein includes an extracellular domain including an extracellular portion of muFas, a transmembrane domain including the transmembrane portion of DR3, and an intracellular domain including an intracellular portion of DR3.

In particular embodiments, a recombinant protein includes an extracellular domain including an extracellular portion of muFas, a transmembrane domain including the transmembrane portion of HVEM, and an intracellular domain including an intracellular portion of HVEM.

In particular embodiments, a recombinant protein includes an extracellular domain including an extracellular portion of muFas, a transmembrane domain including the transmembrane portion of muFas, and an intracellular domain including an intracellular portion of LIGHT.

In particular embodiments, a recombinant protein includes an extracellular domain including an extracellular portion of huCD200R, a 12 amino acid linker, a transmembrane domain including the transmembrane portion of CD28, and an intracellular domain including an intracellular portion of CD2.

In particular embodiments, a recombinant protein includes an extracellular domain including an extracellular portion of huCD200R, a 12 amino acid linker, a transmembrane domain including the transmembrane portion of CD28, and an intracellular domain including an intracellular portion of CD226.

In particular embodiments, a recombinant protein includes an extracellular domain including an extracellular portion of huCD200R, a 12 amino acid linker, a transmembrane domain including the transmembrane portion of CD28, and an intracellular domain including an intracellular portion of CRTAM.

In particular embodiments, a recombinant protein includes an extracellular domain including an extracellular portion of huCD200R, a 12 amino acid linker, a transmembrane domain including the transmembrane portion of CD28, and an intracellular domain including an intracellular portion of HAVCR1.

In particular embodiments, a recombinant protein includes an extracellular domain including an extracellular portion of huCD200R, a 12 amino acid linker, a transmembrane domain including the transmembrane portion of CD28, and an intracellular domain including an intracellular portion of SLAMF3.

In particular embodiments, a recombinant protein includes an extracellular domain including an extracellular portion of huCD200R, a 12 amino acid linker, a transmembrane domain including the transmembrane portion of CD28, and an intracellular domain including an intracellular portion of SLAMF5.

In particular embodiments, a recombinant protein includes an extracellular domain including an extracellular portion of huCD200R, a 12 amino acid linker, a transmembrane domain including the transmembrane portion of CD28, and an intracellular domain including an intracellular portion of SLAMF7.

In particular embodiments, a recombinant protein includes an extracellular domain including an extracellular portion of muCD200R, a transmembrane domain including the transmembrane portion of CD28, and an intracellular domain including an intracellular portion of CD2.

In particular embodiments, a recombinant protein includes an extracellular domain including an extracellular portion of muCD200R, a transmembrane domain including the transmembrane portion of CD28, and an intracellular domain including an intracellular portion of CD226.

In particular embodiments, a recombinant protein includes an extracellular domain including an extracellular portion of muCD200R, a transmembrane domain including the transmembrane portion of CD28, and an intracellular domain including an intracellular portion of CRTAM.

In particular embodiments, a recombinant protein includes an extracellular domain including an extracellular portion of muCD200R, a transmembrane domain including the transmembrane portion of CD28, and an intracellular domain including an intracellular portion of HAVCR1.

In particular embodiments, a recombinant protein includes an extracellular domain including an extracellular portion of muCD200R, a transmembrane domain including the transmembrane portion of CD28, and an intracellular domain including an intracellular portion of SLAMF3.

In particular embodiments, a recombinant protein includes an extracellular domain including an extracellular portion of muCD200R, a transmembrane domain including the transmembrane portion of CD28, and an intracellular domain including an intracellular portion of SLAMF5.

In particular embodiments, a recombinant protein includes an extracellular domain including an extracellular portion of muCD200R, a transmembrane domain including the transmembrane portion of CD28, and an intracellular domain including an intracellular portion of SLAMF7.

In particular embodiments, a recombinant protein includes an extracellular domain including an extracellular portion of huSIRPα, a 12 amino acid linker, a transmembrane domain including the transmembrane portion of CD28, and an intracellular domain including an intracellular portion of CD2.

In particular embodiments, a recombinant protein includes an extracellular domain including an extracellular portion of huSIRPα, a 12 amino acid linker, a transmembrane domain including the transmembrane portion of CD28, and an intracellular domain including an intracellular portion of CD226.

In particular embodiments, a recombinant protein includes an extracellular domain including an extracellular portion of huSIRPα, a 12 amino acid linker, a transmembrane domain including the transmembrane portion of CD28, and an intracellular domain including an intracellular portion of CRTAM.

In particular embodiments, a recombinant protein includes an extracellular domain including an extracellular portion of huSIRPα, a 12 amino acid linker, a transmembrane domain including the transmembrane portion of CD28, and an intracellular domain including an intracellular portion of HAVCR1.

In particular embodiments, a recombinant protein includes an extracellular domain including an extracellular portion of huSIRPα, a 12 amino acid linker, a transmembrane domain including the transmembrane portion of CD28, and an intracellular domain including an intracellular portion of SLAMF3.

In particular embodiments, a recombinant protein includes an extracellular domain including an extracellular portion of huSIRPα, a 12 amino acid linker, a transmembrane domain including the transmembrane portion of CD28, and an intracellular domain including an intracellular portion of SLAMF5.

In particular embodiments, a recombinant protein includes an extracellular domain including an extracellular portion of huSIRPα, a 12 amino acid linker, a transmembrane domain including the transmembrane portion of CD28, and an intracellular domain including an intracellular portion of SLAMF7.

Particular examples of recombinant proteins include (from extracellular domain (ECD) to intracellular domain (ICD)): huFas-huFas tm-CD30 (SEQ ID NO: 64); huFas-huFas tm-HVEM (SEQ ID NO: 65); huFas-huFas tm-LIGHT (SEQ ID NO: 66); muFas-CD30tm-CD30 (SEQ ID NO: 67); muFas-DR3tm-DR3 (SEQ ID NO: 68); muFas-HVEMtm-HVEM (SEQ ID NO: 69); muFas-muFastm-LIGHT (LIGHT is inverted) (SEQ ID NO: 70); huCD200R-12aas-CD28cystm-CD2 (SEQ ID NO: 50); huCD200R-12aas-CD28cys-CD226 (SEQ ID NO: 51); huCD200R-12aas-CD28cys-CRTAM (SEQ ID NO: 52); huCD200R-12aas-CD28cys-HAVCR1 (SEQ ID NO: 53); huCD200R-12aas-CD28cys-SLAMF3 (SEQ ID NO: 54); huCD200R-12aas-CD28cys-SLAMF5 (SEQ ID NO: 55); huCD200R-12aas-CD28cys-SLAMF7 (SEQ ID NO: 56); muCD200R-9aas ec-CD28cys-CD2 (SEQ ID NO: 57); muCD200R-9aas ec-CD28cys-CD226 (SEQ ID NO: 58); muCD200R-9aas ec-CD28cys-CRTAM (SEQ ID NO: 59); muCD200R-9aas ec-CD28cys-HAVCR1 (SEQ ID NO: 60); muCD200R-9aas ec-CD28cys-SLAMF3 (SEQ ID NO: 61); muCD200R-9aas ec-CD28cys-SLAMF5 (SEQ ID NO: 62); muCD200R-9aas ec-CD28cys-SLAMF7 (SEQ ID NO: 63); huSIRPα-12 aas-CD28cystm-CD2 (SEQ ID NO: 71); huSIRPα-12aas-CD28cys-CD226 (SEQ ID NO: 72); huSIRPα-12aas-CD28cys-CRTAM (SEQ ID NO: 73); huSIRPα-12aas-CD28cys-HAVCR1 (SEQ ID NO: 74); huSIRPα-12aas-CD28cys-SLAMF3 (SEQ ID NO: 75); huSIRPα-12aas-CD28cys-SLAMF5 (SEQ ID NO: 76); and/or huSIRPα-12aas-CD28cys-SLAMF7 (SEQ ID NO: 77).

In particular embodiments, a recombinant protein includes an extracellular domain including an extracellular portion of huFas, a transmembrane domain including the transmembrane portion of huFas, and an intracellular domain including an intracellular portion of CD27.

In particular embodiments, a recombinant protein includes an extracellular domain including an extracellular portion of huFas, a transmembrane domain including the transmembrane portion of huFas, and an intracellular domain including an intracellular portion of CD40.

In particular embodiments, a recombinant protein includes an extracellular domain including an extracellular portion of huFas, a transmembrane domain including the transmembrane portion of huFas, and an intracellular domain including an intracellular portion of GITR.

In particular embodiments, a recombinant protein includes an extracellular domain including an extracellular portion of huFas, a transmembrane domain including the transmembrane portion of huFas, and an intracellular domain including an intracellular portion of OX40.

In particular embodiments, a recombinant protein includes an extracellular domain including an extracellular portion of muFas, a transmembrane domain including the transmembrane portion of CD27, and an intracellular domain including an intracellular portion of CD27.

In particular embodiments, a recombinant protein includes an extracellular domain including an extracellular portion of muFas, a transmembrane domain including the transmembrane portion of CD40, and an intracellular domain including an intracellular portion of CD40.

In particular embodiments, a recombinant protein includes an extracellular domain including an extracellular portion of muFas, a transmembrane domain including the transmembrane portion of GITR, and an intracellular domain including an intracellular portion of GITR.

In particular embodiments, a recombinant protein includes an extracellular domain including an extracellular portion of muFas, a transmembrane domain including the transmembrane portion of OX40, and an intracellular domain including an intracellular portion of OX40.

In particular embodiments, a recombinant protein includes an extracellular domain including an extracellular portion of huCD200R, a 12 amino acid linker, a transmembrane domain including the transmembrane portion of CD28, and an intracellular domain including an intracellular portion of ICOS.

In particular embodiments, a recombinant protein includes an extracellular domain including an extracellular portion of huCD200R, a 12 amino acid linker, a transmembrane domain including the transmembrane portion of CD28, and an intracellular domain including an intracellular portion of SLAMF1.

In particular embodiments, a recombinant protein includes an extracellular domain including an extracellular portion of muCD200R, a transmembrane domain including the transmembrane portion of CD28, and an intracellular domain including an intracellular portion of ICOS.

In particular embodiments, a recombinant protein includes an extracellular domain including an extracellular portion of muCD200R, a transmembrane domain including the transmembrane portion of CD28, and an intracellular domain including an intracellular portion of SLAMF1.

In particular embodiments, a recombinant protein includes an extracellular domain including an extracellular portion of huSIRPα, a 12 amino acid linker, a transmembrane domain including the transmembrane portion of CD28, and an intracellular domain including an intracellular portion of ICOS.

In particular embodiments, a recombinant protein includes an extracellular domain including an extracellular portion of huSIRPα, a 12 amino acid linker, a transmembrane domain including the transmembrane portion of CD28, and an intracellular domain including an intracellular portion of SLAMF1.

Particular examples of recombinant proteins include (from extracellular domain (ECD) to intracellular domain (ICD)): huFas-huFas tm-CD27 (SEQ ID NO: 101); huFas-huFas tm-CD40 (SEQ ID NO: 102); huFas-huFas tm-GITR (SEQ ID NO: 103); huFas-huFas tm-OX40 (SEQ ID NO: 104); muFas-CD27tm-CD27 (SEQ ID NO: 105); muFas-CD40tm-CD40 (SEQ ID NO: 106); muFas-GITRtm-GITR (SEQ ID NO: 107); muFas-OX40tm-OX40 (SEQ ID NO: 108); huCD200R-12aas-CD28cys-ICOS (SEQ ID NO: 97); huCD200R-12aas-CD28cys-SLAMF1 (SEQ ID NO: 98); muCD200R-9aas ec-CD28cys-ICOS (SEQ ID NO: 99); muCD200R-9aas ec-CD28cys-SLAMF1 (SEQ ID NO: 100); huSIRPα-12aas-CD28cys-ICOS (SEQ ID NO: 109); and/or huSIRPα-12aas-CD28cys-SLAMF1 (SEQ ID NO: 110).

The current disclosure also provides selecting particular recombinant proteins disclosed herein, and particular combinations of recombinant proteins disclosed herein, based on distinct attributes of these proteins and the clinical needs of patients. For example, as disclosed herein Fas-CD40 is particularly useful to stimulate cytokine production; Fas-OX40 is particularly useful to drive T cell expansion; Fas-CD27 is particularly useful to drive T cell proliferation, while Fas-CDHVEM and SIRPα-ICOS are particularly effective at tumor cell lysis. Thus, if a patient were experiencing T cell exhaustion, a combination of Fas-CD40, Fas-OX40, Fas-CD27, and Fas-HVEM may be selected. A combination of Fas-CD40, Fas-CD27, and Fas-HVEM may also be selected.

Particular embodiments include selecting Fas-CD40 to stimulate cytokine production. Particular embodiments include selecting Fas-OX40 to stimulate T cell expansion. Particular embodiments include selecting Fas-CD27 to stimulate T cell proliferation. Particular embodiments include selecting Fas-HVEM and/or SIRPα-ICOS to stimulate tumor cell lysis.

Certain examples utilize a combination of Fas-CD40 and Fas-OX40, Fas-CD40 and Fas-HVEM, Fas-CD40 and SIRPα-ICOS, Fas-OX40 and Fas-HVEM, Fas-OX40 and SIRPα-ICOS, or Fas-HVEM and SIRPα-ICOS. Certain examples utilize a combination of Fas-CD40 and Fas-CD27, Fas-CD27 and Fas-HVEM, or Fas-CD27 and SIRPα-ICOS.

Certain examples utilize a combination of Fas-CD40, Fas-CD27, and Fas-HVEM; Fas-CD40, Fas-CD27, and SIRPα-ICOS; Fas-CD27, Fas-HVEM, and SIRPα-ICOS. Certain examples utilize a combination of Fas-CD40, Fas-OX40, and Fas-HVEM; Fas-CD40, Fas-OX40, and SIRPα-ICOS; Fas-OX40, Fas-HVEM, and SIRPα-ICOS.

Aspects of the current disclosure are now described in more supporting detail as follows: (i) Multimerization Systems; (ii) Genes & Gene Delivery Techniques; (iii) Ex Vivo Cell Manufacturing; (iv) Formulations & Compositions; (v) Nanoparticles for In Vivo Nucleotide Delivery to Immune Cells; (vi) Methods of Use; (vii) Exemplary Embodiments; and (viii) Closing Paragraphs. These headings are provided for organizational purposes only and do not limit the scope or interpretation of the disclosure.

(i) Multimerization Systems. Recombinant proteins disclosed herein can include multimerization domains that result in dimers, trimers, tetramers, hexamers, heptamers, octamers etc. for example, using coiled-coil multimerization domains.

Particular embodiments include chemically-induced multimerization systems (CIMS). The chemical inducer of multimerization (CIM) (e.g., dimerization, trimerization) and CIM binding domains may be any combination of molecules, peptides, or domains which enable the selective co-localization and multimerization of recombinant proteins in the presence of the CIM. For example, in particular embodiments, one recombinant proteins includes a CIM binding domain 1 (CBM1) and the second recombinant protein contains the second CIM binding domain (CBM2). CBM1 and CBM2 are capable of simultaneously binding to the CIM. The CIM may interact with CBMs in which CBM1 and CBM2 are identical or the CIM may interact with two different CBMs so that CBM1 and CBM2 are not identical.

Small molecule CIMS for facilitating the co-localization of proteins are known in the art (Corson et al.; 2008; ACS Chemical Biology; 3(11); 667). The CIM and CBMs may be the FK506 binding protein (FKBP) ligand dimerization system described by Clackson et al. (PNAS; 1998; 95; 10437-10442). This dimerization system includes two FKBP-like binding domains with a F36V mutation in the FKBP binding domain and a CID dimerization agent (AP1903) with complementary amino acid substitutions. Exposing cells engineered to express FKBP-like binding domain fusion proteins to AP103 results in the dimerization of the proteins including the FKBP-like binding domains but no interactions involving endogenous FKBP.

The CIM/CIM binding domain may also be the rapamycin and FKBP12/FKBP12-Rapamycin Binding (FRB) domain of the mTOR system described by Rivera et al. (Nature Med; 1996; 2; 1028-1032) or the non-immunosuppressive rapamycin analogs (rapalogs) and FKBP12/FRB system described by Bayle et al. (Chem Bio; 2006; 13; 99-107). For example the CIM may be C-20-methyllyrlrapamycin (MaRap) or C16(S)-Butylsulfonamidorapamycin (C16-BS-Rap). The CIM may be C16-(S)-3-methylindolerapamycin (C16-iRap) or C16-(S)-7-methylindolerapamycin (AP21976/C16-AiRap) in combination with the respective complementary binding domains for each.

The CIM and CBMs may include the dimerization system described by Belshaw et al. (Nature; 1996; 93; 4604-4607), which utilizes a FK506 (Tacrolimus)/cyclosporin fusion molecule as the CIM agent with FK-binding protein 12 (FKBP12) and cylcophilin A as the CBMs.

Bacterial DNA gyrase B (GyrB) binding domains can be used as CBMs within a dimerization system with the antibiotic coumermycin as the CIM (Farrar et al., Methods Enzymol; 2000; 327; 421-419 and Nature; 1996; 383; 178-181).

Other dimerization systems include an estrone/biotin CIM in combination with an oestrogen-binding domain (EBD) and a streptavidin binding domain (Muddana & Peterson; Org. Lett; 2004; 6; 1409-1412; Hussey et al.; J. Am. Chem. Soc.; 125; 3692-3693) and a dexamethasone/methotrexate CIM in combination with a glucocorticoid-binding domain (GBD) and a dihydrofolate reductase (DHFR) binding domain (Lin et al.; J. Am. Chem. Soc.; 2000; 122; 4247-4248). RSL1 or a derivative thereof can be used as a CIM in the heterodimerization of molecules with CBMs made up of EcR and RXR domains. Other examples of dimerization systems include a CIM in which the methotrexate portion of the CIM is replaced with the bacterial specific DHFR inhibitor trimethoprim (Gallagher et al.; Anal. Biochem; 2007; 363; 160-162) and an O⁶-benzylguanine derivative/methotrexate CIM in combination with an O6-alkylguanine-DNA alkyltransferase (AGT) binding domain and a DHFR binding domain (Gendreizig et al.; J. Am. Chem. Soc.; 125; 14970-14971).

Trimerization systems can be engineered similarly to dimerization systems. For example, a chemically inducible trimerization domain can be engineered by splitting FRB and/or FKBP. Efficient trimerization of split pairs of FRB or FKBP with full-length FKBP or FRB, respectively by rapamycin is described in Wu, et al., Nature Methods 17, 928-936, 2020.

Coiled-coil multimerization domains are composed of interacting, amphipathic a helices characterized by a seven-residue repeat sequence (a heptad repeat), a.b.c.d.e.f.g, with hydrophobic residues predominant at positions a and d (positions one and four), and polar residues generally elsewhere (Harbury et al., Science 262:1401 (1993)). The leucine zipper domains are coiled-coil domains that typically have leucine at the d position of the heptad repeats. Naturally occurring, coiled-coils are typically made up of multiple heptad repeats, for example, three or more sequences, (a.b.c.d.e.f.g)1-(a.b.c.d.e.f.g)2-(a.b.c.d.e.f.g)3, etc. The designation “(a.b.c.d.e.f.g) n” merely refers to two or more additional half (3-4 amino acids) or full length (7 amino acids) heptad repeat sequences, where each half or full (a.b.c.d.e.f.g.) repeat need not have the identical amino acid sequence.

(ii) Genes & Gene Delivery Techniques. Immune cells are modified to express a recombinant protein of the disclosure by delivering a nucleotide including a gene that encodes the recombinant protein. A gene is a distinct sequence of nucleotides, the order of which determines the order of monomers in a polypeptide or nucleic acid molecule which a cell (or virus) may synthesize. The term “gene” may include not only coding sequences but also regulatory regions such as promoters, enhancers, and termination regions.

A promoter is a region of DNA, generally upstream (5′) of a coding region, which controls at least in part the initiation and level of gene transcription. Promoters generally extend upstream from a transcription initiation site and are involved in the binding of RNA polymerase. Promoters may contain several short (<<10 base pair) sequence elements that bind transcription factors, generally dispersed over >>200 base pairs. The term promoter includes inducible and constitutive promoters. Particular embodiments disclosed herein utilize inducible promoters.

An inducible promoter refers to a promoter whose activity can be increased or decreased upon an external stimulus. Stimuli can be chemical or physical in nature, such as by administration of a chemical or by adjustment of temperature or light.

Chemically inducible promoters include reproductive hormone induced promoters and antibiotic inducible promoters such as the tetracycline inducible promoter and the zinc-inducible metallothionine promoter.

An example of a chemically inducible system includes the Tet-Off™ or Tet-On™ system (Clontech, Palo Alto, Calif.). This system allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. coli.

Other inducible promoter systems include the Lac operator repressor system inducible by IPTG (isopropyl beta-D-thiogalactoside) (Cronin, A. et al. 2001. Genes and Development, v. 15); ecdysone-based inducible systems (Hoppe, U. C. et al. 2000. Mol. Ther. 1:159-164); estrogen-based inducible systems (Braselmann, S. et al. 1993. Proc. Natl. Acad. Sci. 90:1657-1661); progesterone-based inducible systems; and CID-based inducible systems. A progesterone-based inducible system, for example, uses a chimeric regulator, GLVP, which is a hybrid protein including the GAL4 binding domain and the herpes simplex virus transcriptional activation domain, VP16, and a truncated form of the human progesterone receptor that retains the ability to bind ligand and can be turned on by RU486 (Wang, et al. 1994. Proc. Natl. Acad. Sci. 91:8180-8184). As indicated previously, a CID-based inducible system, for example, uses CIDs to regulate gene expression, such as a system wherein rapamycin induces dimerization of the cellular proteins FKBP12 and FRAP (Belshaw, P. J. et al. 1996. J. Chem. Biol. 3:731-738; Fan, L. et al. 1999. Hum. Gene Ther. 10:2273-2285; Shariat, S. F. et al. 2001. Cancer Res. 61:2562-2571; Spencer, D. M. 1996. Curr. Biol. 6:839-847). Chemical substances that activate the chemically inducible promoters can be administered to a cell or subject containing the gene of interest via any method known to those of skill in the art.

Temperature inducible promoters are induced to prompt expression with exposure to either heat or cold. Temperature inducible promoters include heat shock-inducible Hsp70 or Hsp90-derived promoters which prompt expression due to a brief heat shock.

Light inducible promoters use light to regulate transcription. For example, red flame plasmid pDawn contains the blue-light sensing protein YFI. When light is present, YFI is inactive. Without light, YFI phosphorylates FixJ, which binds to the FixK2 promoter to induce transcription of the phage repressor cl, inhibiting transcription from phage promoter pR to prevent expression of a reporter gene.

Constitutive promoters are unregulated promoters that allow for continual transcription of genes. Constitutive promoters include immediate early cytomegalovirus (CMV) promoter, herpes simplex virus 1 (HSV1) immediate early promoter, SV40 promoter, lysozyme promoter, early and late CMV promoters, early and late HSV promoters, β-actin promoter, tubulin promoter, and Rous-Sarcoma virus (RSV) promoter.

An enhancer is a cis-acting sequence that increases the level of transcription associated with a promoter, and can function in either orientation relative to the promoter and the coding sequence that is to be transcribed, and can be located upstream or downstream relative to the promoter or the coding sequence to be transcribed.

Other regulatory elements include: silencers, insulators, and locus control regions. Silencers are opposite to enhancers in that they are capable of binding transcription regulation factors call repressors and thus reduce the level of transcription of a target gene. Insulators limit inappropriate interactions between adjacent domains. Insulators prevent regulatory elements from having an effect on domains outside the domain of the regulatory element. Locus control regions (LCRs) are long-range cis-regulatory elements that control expression of an entire set of linked genes on a locus.

Nucleotides with genes encoding recombinant proteins can be delivered to immune cells using any technique known to those of ordinary skill in the art, such as through electroporation, viral vectors, and nanoparticles.

In particular embodiments, a vector can be used to deliver nucleotides to cells. A vector is any nucleic acid vehicle (DNA or RNA) capable of facilitating the transfer of a nucleotide of interest into cells. In general, vectors include plasmids, phagemids, viral vectors, and other vehicles derived from viral or bacterial sources that have been manipulated by the insertion or incorporation of a target nucleotide sequence.

In particular embodiments, nucleotides can be delivered by electroporation in which an electrical field is applied to cells in order to increase their permeability. Instruments that can be used for electroporation include the Neon transfection system (Thermo Fisher Scientific, Waltham, MA), Gemini instrument and AgilePulse/CytoPulse instrument (BTX-Harvard apparatus, Holliston, MA), 4D-Nucleofector system, Amaxa Nucleofector II, Nucleofector 2b instrument (Lonza, Switzerland), CTX-1500A instrument (Celetrix, Manassas, VA), MaxCyte GT or VLX instrument (MaxCyte, Gathersbur, MD), and Gene Pulser Xcell (Biorad, Hercules, CA).

In particular embodiments, viral vectors can be used to deliver nucleotides to immune cells. Viral vectors can include any non-cytopathic eukaryotic virus in which nonessential genes have been replaced with the target nucleotide sequence to be delivered. Non-cytopathic viruses include lentivirus; adenovirus; adeno-associated virus (AAV); SV40-type viruses; polyoma viruses; Epstein-Barr viruses; papilloma viruses; herpes virus; vaccinia virus; and polio virus. One can readily employ other vectors not named but known to the art.

Nanoparticles can be used to selectively deliver nucleotides to immune cells ex vivo or in vivo, as described more fully below. Targeted nanoparticles capable of in vivo delivery can take many forms and will generally include a cell-specific targeting ligand (e.g., derived from an antibody binding domain that binds a marker of an immune-targeted cell). For example, all T cells express CD3 whereas helper T cells express CD4 and cytotoxic T cells express CD8+. Numerous additional immune cell surface markers are known to those of ordinary skill and the art and can be used for targeted nanoparticle delivery.

Nanoparticles can be formed in a variety of different shapes, including spheroidal, cuboidal, pyramidal, oblong, cylindrical, toroidal, and the like. Nucleotides can be encapsulated within nanoparticles and/or covalently and/or non-covalently bound to the surface or close underlying vicinity of the surface of the nanoparticle.

Liposomes are microscopic vesicles including at least one concentric lipid bilayer that surrounds an aqueous core. In certain examples, the structure of a liposome can be used to encapsulate a nanoparticle within its core (i.e., a liposomal nanoparticle). Lipid nanoparticles (LNPs) are liposome-like structures that lack the continuous lipid bilayer characteristic of liposomes. Solid lipid nanoparticles (SLNs) are LNPs that are solid at room and body temperatures. Liposomes and similar structures can be neutral (cholesterol) or bipolar and include phospholipids.

Methods of forming liposomes are described in, for example, U.S. Pat. Nos. 4,229,360; 4,224,179; 4,241,046; 4,737,323; 4,078,052; 4,235,871; 4,501,728; and 4,837,028, as well as in Szoka et al., Ann. Rev. Biophys. Bioeng. 9:467 (1980) and Hope et al., Chem. Phys. Lip. 40:89 (1986).

Nanoparticles can also be formulated from configurations of positively-charged and neutral or negatively-charged polymers. Examples of positively charged polymers include polyamines; polyorganic amines (e.g., polyethyleneimine (PEI), polyethyleneimine celluloses); poly (amidoamines) (PAMAM); and polyamino acids (e.g., polylysine (PLL), polyarginine). Examples of neutrally charged polymers include polyethylene glycol (PEG); poly (propylene glycol); and polyalkylene oxide copolymers, (PLURONIC®, BASF Corp., Mount Olive, NJ).

Blends of polymers in any concentration and in any ratio can also be used. Blending different polymer types in different ratios using various grades can result in characteristics that borrow from each of the contributing polymers. Various terminal group chemistries can also be adopted.

The size of nanoparticles can vary and can be measured in different ways. In certain examples, nanoparticles have a minimum dimension of equal to or less than 500 nm, less than 150 nm, less than 140 nm, less than 120 nm, less than 110 nm, less than 100 nm, less than 90 nm, less than 80 nm, less than 70 nm, less than 60 nm, less than 50 nm, less than 40 nm, less than 30 nm, less than 20 nm, or less than 10 nm.

Particular embodiments may also use targeted genetic engineering systems to insert delivered nucleotides into a targeted region of the genome. Such systems include the CRISPR (Clustered Regularly Interspaced Short Palindromic Repeats)/Cas (CRISPR-associated protein) nuclease system, zinc finger nucleases (ZFNs), transcription activator-like effector nulceases (TALENs), or MegaTALs having a single chain rare-cleaving nuclease structure in which a TALE is fused with the DNA cleavage domain of a meganuclease.

(iii) Ex Vivo Cell Manufacturing. Immune cells can be obtained from a number of sources, including peripheral blood, mobilized peripheral blood, bone marrow, lymph node tissue, spleen tissue, and tumors. In certain embodiments, immune cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis or leukapheresis product typically contains lymphocytes, including T cells, monocytes, granulocytes, B cells, other nucleated white blood cells, red blood cells, and platelets. In one embodiment, the cells collected by apheresis may be washed to remove the plasma fraction and the cells may be placed in an appropriate buffer or media for subsequent processing. In one embodiment, the cells are washed with phosphate buffered saline (PBS). In an alternative embodiment, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

A specific subpopulation of immune cells can be isolated by positive or negative selection techniques. For example, immune cells can be isolated by incubation with antibody-conjugated beads (e.g., specific for any marker described herein), such as DYNABEADS® (Life Technologies AS, Oslo, Norway) for a time period sufficient for positive selection of the desired immune cells. In particular embodiments, the time period ranges from 30 minutes to 36 hours. In particular embodiments, the time period is 30 minutes, 1 hour, 2 hours, 3 hours, 4 hours, 5 hours, or 6 hours. Longer incubation times may be used to isolate immune cells in any situation where there are few immune cells as compared to other cell types, such in isolating tumor infiltrating lymphocytes (TIL) from tumor tissue or from immunocompromised individuals. Further, use of longer incubation times can increase the efficiency of capture of CD8+ T cells. Immune cells can be selected based on a biomarker on the cell surface including CD3, CD4, CD8, TIM-3, LAG-3, 4-1BB, or PD-1.

Enrichment of an immune cell population by positive or negative selection can be accomplished with a combination of antibodies directed to surface markers unique to the positively or negatively selected cells. One method is to use cell sorting and/or selection via negative magnetic immunoadherence or flow cytometry that uses a cocktail of monoclonal antibodies directed to cell surface markers present on the cells negatively selected. Sorting of immune cells, or generally any cells, can be carried out using any of a variety of commercially available cell sorters, including MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

The efficiency of the purification can be analyzed by flow cytometry (Coulter, EPICS Elite), using, for example, anti-CD3, anti-CD4, anti-CD8, anti-CD14 mAbs or additional antibodies that recognize specific subsets of T cells, followed by fluorescein isothiocyanate conjugated goat anti mouse immunoglobulin (Fisher, Pittsburgh, PA) or other secondary antibody.

The isolated cells can be expanded in a culture media under specific conditions. In particular embodiments, the isolated cells are cultured in the presence of IL-2. In particular embodiments, the isolated cells can be expanded using methods described in U.S. Pat. No. 8,637,307. The numbers of immune cells may be increased at least 3-fold (or 4-, 5-, 6-, 7-, 8-, or 9-fold), more preferably at least 10-fold (or 20-, 30-, 40-, 50-, 60-, 70-, 80-, or 90-fold), more preferably at least 100-fold, more preferably at least 1,000 fold, or most preferably at least 100,000-fold. The numbers of immune cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133.

Expanded cells may be activated in culture utilizing appropriate stimulating ligands (e.g., with CD3/CD28 beads useful for stimulating the CD3 primary signal and the CD28 accessory or co-stimulatory signal). Activating ligands may be soluble or immobilized on a surface.

(iv) Formulations & Compositions. Exemplary carriers for cell formulations for administration to a subject include saline, buffered saline, physiological saline, water, Hanks' solution, Ringer's solution, Normosol-R (Abbott Labs), Plasma-Lyte A® (Baxter Laboratories, Inc., Morton Grove, IL), glycerol, ethanol, and combinations thereof.

Therapeutically effective amounts of cells within cell-based formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

In particular embodiments, cell formulations can be administered to subjects as soon as reasonably possible following their initial formulation. In particular embodiments, cell formulations can be frozen or cryopreserved. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen. Prior to administration to a subject, the frozen cell formulations are thawed.

(v) Nanoparticles for In Vivo Nucleotide Delivery to Immune Cells. As described previously, nanoparticles can be used to selectively deliver nucleotides to immune cells in vivo. In this scenario, nanoparticles can be formulated into compositions for delivery with a pharmaceutically acceptable carrier that is suitable for administration to a subject. Pharmaceutically acceptable carriers include those that do not produce significantly adverse, allergic or other untoward reactions that outweigh the benefit of administration, whether for research, prophylactic and/or therapeutic treatments. Exemplary pharmaceutically acceptable carriers and formulations are disclosed in Remington's Pharmaceutical Sciences, 18th Ed. Mack Printing Company, 1990. Moreover, compositions can be prepared to meet sterility, pyrogenicity, general safety and purity standards as required by United States FDA Office of Biological Standards and/or other relevant foreign regulatory agencies.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, chelating agents (e.g., EDTA), gels, binders, disintegration agents, and/or lubricants.

For injection, compositions can be made as aqueous solutions, such as in buffers such as Hanks' solution, Ringer's solution, or physiological saline. The solutions can contain formulatory agents such as suspending, stabilizing and/or dispersing agents. Alternatively, the composition can be in lyophilized and/or powder form for constitution with a suitable vehicle, e.g., sterile pyrogen-free water, before use.

Therapeutically effective amounts of nanoparticles within a composition can include at least 0.1% w/v or w/w particles; at least 1% w/v or w/w particles; at least 10% w/v or w/w particles; at least 20% w/v or w/w particles; at least 30% w/v or w/w particles; at least 40% w/v or w/w particles; at least 50% w/v or w/w particles; at least 60% w/v or w/w particles; at least 70% w/v or w/w particles; at least 80% w/v or w/w particles; at least 90% w/v or w/w particles; at least 95% w/v or w/w particles; or at least 99% w/v or w/w particles.

(vi) Methods of Use. Methods disclosed herein include treating subjects (humans, veterinary animals (dogs, cats, reptiles, birds, etc.) livestock (horses, cattle, goats, pigs, chickens, etc.) and research animals (monkeys, rats, mice, fish, etc.) with ex vivo manufactured cell formulations or nanoparticle compositions disclosed herein. Treating subjects includes delivering therapeutically effective amounts. Therapeutically effective amounts include those that provide effective amounts, prophylactic treatments and/or therapeutic treatments.

An “effective amount” is the amount of a formulation or composition necessary to result in a desired physiological effect. Effective amounts are often administered for research purposes. Effective amounts disclosed herein can cause an indication of immune cell activation in an in vitro assessment or within an in vivo animal model. Indications of T cell activation include cytokine release, upregulated activation (e.g. expression of CD69, CD25, etc.), tumor cell lysis, proliferation, and accumulation.

A “prophylactic treatment” includes a treatment administered to a subject who does not display signs or symptoms of a condition (e.g., cancer or an infection) or displays only early signs or symptoms of the condition such that treatment is administered for the purpose of diminishing or decreasing the risk of developing the condition further. Thus, a prophylactic treatment functions as a preventative treatment against a condition. In particular embodiments, prophylactic treatments reduce, delay, or prevent the worsening of a condition.

A “therapeutic treatment” includes a treatment administered to a subject who displays symptoms or signs of a condition and is administered to the subject for the purpose of diminishing or eliminating those signs or symptoms of the condition. The therapeutic treatment can reduce, control, or eliminate the presence or activity of the condition and/or reduce control or eliminate side effects of the condition.

Function as an effective amount, prophylactic treatment or therapeutic treatment are not mutually exclusive, and in particular embodiments, administered dosages may accomplish more than one treatment type.

In particular embodiments, therapeutically effective amounts provide anti-cancer effects. Anti-cancer effects include a decrease in the number of cancer cells, decrease in the number of metastases, a decrease in tumor volume, an increase in life expectancy, induced chemo-or radiosensitivity in cancer cells, inhibited angiogenesis near cancer cells, inhibited cancer cell proliferation, inhibited tumor growth, prevented or reduced metastases, prolonged subject life, reduced cancer-associated pain, and/or reduced relapse or re-occurrence of cancer following treatment.

A “tumor” can be liquid or solid depending on the cell origin. A solid tumor is a swelling or lesion formed by an abnormal growth of cells (called neoplastic cells or tumor cells). A “tumor cell” is an abnormal cell that grows by a rapid, uncontrolled cellular proliferation and continues to grow after the stimuli that initiated the new growth cease and can be considered a solid tumor or liquid tumor in the art depending on the cell origin. Tumors show partial or complete lack of structural organization and functional coordination with the normal tissue, and usually form a distinct mass of tissue, which may be benign, pre-malignant or malignant. Liquid tumors refer to the total mass of circulating neoplastic cells, for examples in hematopoietic malignancies such as leukemia.

In particular embodiments, therapeutically effective amounts provide anti-infection effects. Infections may be viral, bacterial, fungal, protozoan, parasitic, or prion infections. Examples of viral diseases include measles, rubella, COVID-19, chickenpox/shingles, roseola, smallpox, and influenza. Examples of bacteria that cause infections include Streptococcus, Staphylococcus, Tuberculosis, Salmonella, and Escherichia coli. Examples of fungal infections include Histoplasmosis, Blastomycosis, Coccidioidomycosis, Paracoccidioidomycosis, Aspergillosis, Candidiasis, and Mucormycosis. Examples of parasitic infections include toxoplasmosis, giardiasis, cryptosporidiosis, and trichomoniasis. Examples of prion diseases include Creutzfeldt-Jakob Disease, Variant Creutzfeldt-Jakob Disease, Fatal Familial Insomnia, Kuru, Gerstmann-Straussler-Schneinker Syndrome, Bovine Spongiform, and Chronic Wasting Disease.

In particular embodiments, modified cells described herein may be used for adoptive cell transfer (ACT). Adoptive cell transfer can include: isolating from a biological sample of the subject an immune cell or immune cell population; in vitro expanding and modifying the immune cell or immune cell population to express a gene (e.g., a gene encoding a recombinant protein described herein and optionally one or more additional therapeutic molecules); and administering the in vitro expanded/modified immune cell or immune cell population to the subject. The method may further include enriching the expanded immune cells for one subtype. In certain embodiments, the method may further include formulating the in vitro expanded immune cell or immune cell population into a cell-based formulation. In this manner, ACT refers to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma and colorectal carcinoma, as well as patients with CD19-expressing hematologic malignancies (Kalos et al., (2011) Science Translational Medicine 3 (95): 95ra73).

In certain embodiments, cells modified to express a recombinant protein disclosed herein provide an anti-cancer or anti-infection treatment by providing enhanced immune system activation. In certain embodiments, cells modified to express a protein disclosed herein provide an anti-cancer or anti-infection treatment in combination with a cancer treatment or infection treatment.

In certain embodiments, a cell genetically modified to express a recombinant protein disclosed herein is additionally genetically modified to express a CAR or TCR. The CAR or TCR can bind a cancer antigen or a viral antigen.

Exemplary cancer antigens include bladder cancer antigens: MUC16, PD-L1, EGFR; breast cancer antigens: HER2, ERBB2, ROR1, PD-L1, EGFR, MUC16, FOLR, CEA, p53; cholangiocarcinoma antigens: mesothelin, PD-L1, EGFR; colorectal cancer antigens: CEA, PD-L1, EGFR, K-ras; glioblastoma antigens: EGFR variant III (EGFRvIII), IL13Ra2; lung cancer antigens: ROR1, PD-L1, EGFR, mesothelin, MUC16, FOLR, CEA, CD56, p53, Kras; Merkel cell carcinoma antigens: CD56, PD-L1, EGFR; mesothelioma antigens: mesothelin, PD-L1, EGFR; neuroblastoma antigens: ROR1, glypican-2, CD56, disialoganglioside, PD-L1, EGFR; ovarian cancer antigens: EpCam, L1-CAM, MUC16, folate receptor (FOLR), Lewis Y, ROR1, mesothelin, WT-1, PD-L1, EGFR, CD56, p53; melanoma antigens: Tyrosinase related protein 1 (TYRP1/gp75); GD2, PD-L1, EGFR; multiple myeloma antigens: B-cell maturation antigen (BCMA), PD-L1, EGFR; pancreatic cancer antigens: mesothelin, CEA, CD24, ROR1, PD-L1, EGFR, MUC16, K-ras; prostate cancer antigens: PSMA, WT1, Prostate Stem Cell antigen (PSCA), SV40 T, PD-L1, EGFR; renal cell carcinoma antigens: carboxy-anhydrase-IX (CAIX); PD-L1, EGFR; and stem cell cancer antigens: CD133, PD-L1, EGFR.

Exemplary viral antigens include coronaviral antigens: the spike(S) protein; cytomegaloviral antigens: envelope glycoprotein B and CMV pp65; Epstein-Barr antigens: EBV EBNAI, EBV P18, and EBV P23; hepatitis antigens: the S, M, and L proteins of hepatitis B virus, the pre-S antigen of hepatitis B virus, HBCAG DELTA, HBV HBE, hepatitis C viral RNA, HCV NS3 and HCV NS4; herpes simplex viral antigens: immediate early proteins and glycoprotein D; HIV antigens: gene products of the gag, pol, and env genes such as HIV gp32, HIV gp41, HIV gp120, HIV gp160, HIV P17/24, HIV P24, HIV P55 GAG, HIV P66 POL, HIV TAT, HIV GP36, the Nef protein and reverse transcriptase; influenza antigens: hemagglutinin and neuraminidase; Japanese encephalitis viral antigens: proteins E, M-E, M-E-NS1, NS1, NS1-NS2A and 80% E; measles antigens: the measles virus fusion protein; rabies antigens: rabies glycoprotein and rabies nucleoprotein; respiratory syncytial viral antigens: the RSV fusion protein and the M2 protein; rotaviral antigens: VP7sc; rubella antigens: proteins E1 and E2; and varicella zoster viral antigens: gpl and gpll. See Fundamental Virology, Second Edition, eds. Fields, B. N. and Knipe, D. M. (Raven Press, New York, 1991) for additional examples of viral antigens.

Certain examples include obtaining a sample from a subject and assessing the subject's T cells for proliferation, cytokine production, or tumor cell lysis. Based on the assessment, a particular recombinant protein disclosed herein may be selected for the subject. For example, if a subject's T cells show low cytokine production, Fas-CD40 may be selected as a recombinant protein for the subject. If a subject's T cells show low proliferation, Fas-CD27 may be selected as a recombinant protein for the subject. If a subject's T cells show low tumor cell lysis, Fas-HVEM and/or SIRPα-ICOS may be selected as a recombinant protein for the subject. Pairs and combinations of recombinant proteins may also be selected based on an assessment of the subject's clinical status and/or an assessment of the subject's T cell function.

### (vii) Exemplary Embodiments

1. A protein including
an extracellular domain of Fas, CD200R, or SIRPα;
an intracellular domain of HVEM, CD2, CD226, CRTAM, HAVCR1, SLAMF3, SLAMF5, SLAMF7, DR3, CD30, or LIGHT; and
a transmembrane domain linking the extracellular domain to the intracellular domain.
2. The protein of embodiment 1, wherein
the extracellular domain is an extracellular domain of Fas and the intracellular domain is an intracellular domain of HVEM, CD30, DR3, LIGHT, CD2, CD226, CRTAM, HAVCR1, SLAMF3, SLAMF5, or SLAMF7; or
the extracellular domain is an extracellular domain of CD200R and the intracellular domain is an intracellular domain of CD2, CD226, CRTAM, HAVCR1, SLAMF3, SLAMF5, SLAMF7, CD30, DR3, HVEM, or LIGHT;
the extracellular domain is an extracellular domain of SIRPα and the intracellular domain is an intracellular domain of CD2, CD226, CRTAM, HAVCR1, SLAMF3, SLAMF5, SLAMF7, CD30, DR3, HVEM, or LIGHT.
3. The protein of embodiment 1 or 2, wherein
the extracellular domain includes an extracellular domain of Fas and the intracellular domain includes an intracellular domain of HVEM;
the extracellular domain includes an extracellular domain of Fas and the intracellular domain includes an intracellular domain of CD2;
the extracellular domain includes an extracellular domain of Fas and the intracellular domain includes an intracellular domain of CD226;
the extracellular domain includes an extracellular domain of Fas and the intracellular domain includes an intracellular domain of CRTAM;
the extracellular domain includes an extracellular domain of Fas and the intracellular domain includes an intracellular domain of HAVCR1;
the extracellular domain includes an extracellular domain of Fas and the intracellular domain includes an intracellular domain of SLAMF3;
the extracellular domain includes an extracellular domain of Fas and the intracellular domain includes an intracellular domain of SLAMF5;
the extracellular domain includes an extracellular domain of Fas and the intracellular domain includes an intracellular domain of SLAMF7;
the extracellular domain includes an extracellular domain of Fas and the intracellular domain includes an intracellular domain of CD30;
the extracellular domain includes an extracellular domain of Fas and the intracellular domain includes an intracellular domain of DR3;
the extracellular domain includes an extracellular domain of Fas and the intracellular domain includes an intracellular domain of LIGHT;
the extracellular domain includes an extracellular domain of CD200R and the intracellular domain includes an intracellular domain of CD2;
the extracellular domain includes an extracellular domain of CD200R and the intracellular domain includes an intracellular domain of CD226;
the extracellular domain includes an extracellular domain of CD200R and the intracellular domain includes an intracellular domain of CRTAM;
the extracellular domain includes an extracellular domain of CD200R and the intracellular domain includes an intracellular domain of HAVCR1;
the extracellular domain includes an extracellular domain of CD200R and the intracellular domain includes an intracellular domain of SLAMF3;
the extracellular domain includes an extracellular domain of CD200R and the intracellular domain includes an intracellular domain of SLAMF5;
the extracellular domain includes an extracellular domain of CD200R and the intracellular domain includes an intracellular domain of SLAMF7;
the extracellular domain includes an extracellular domain of CD200R and the intracellular domain includes an intracellular domain of CD30;
the extracellular domain includes an extracellular domain of CD200R and the intracellular domain includes an intracellular domain of DR3;
the extracellular domain includes an extracellular domain of CD200R and the intracellular domain includes an intracellular domain of HVEM;
the extracellular domain includes an extracellular domain of CD200R and the intracellular domain includes an intracellular domain of LIGHT;
the extracellular domain includes an extracellular domain of SIRPα and the intracellular domain includes an intracellular domain of CD2;
the extracellular domain includes an extracellular domain of SIRPα and the intracellular domain includes an intracellular domain of CD226;
the extracellular domain includes an extracellular domain of SIRPα and the intracellular domain includes an intracellular domain of CRTAM;
the extracellular domain includes an extracellular domain of SIRPα and the intracellular domain includes an intracellular domain of HAVCR1;
the extracellular domain includes an extracellular domain of SIRPα and the intracellular domain includes an intracellular domain of SLAMF3;
the extracellular domain includes an extracellular domain of SIRPα and the intracellular domain includes an intracellular domain of SLAMF5;
the extracellular domain includes an extracellular domain of SIRPα and the intracellular domain includes an intracellular domain of SLAMF7;
the extracellular domain includes an extracellular domain of SIRPα and the intracellular domain includes an intracellular domain of CD30;
the extracellular domain includes an extracellular domain of SIRPα and the intracellular domain includes an intracellular domain of DR3;
the extracellular domain includes an extracellular domain of SIRPα and the intracellular domain includes an intracellular domain of HVEM; or the extracellular domain includes an extracellular domain of SIRPα and the intracellular domain includes an intracellular domain of LIGHT.
4. The protein of any of embodiments 1-3, wherein
the extracellular domain includes an extracellular domain of Fas, the intracellular domain includes an intracellular domain of HVEM, and the transmembrane domain includes a transmembrane domain of Fas;
the extracellular domain includes an extracellular domain of Fas, the intracellular domain includes an intracellular domain of HVEM, and the transmembrane domain includes a transmembrane domain of HVEM;
the extracellular domain includes an extracellular domain of Fas, the intracellular domain includes an intracellular domain of CD30, and the transmembrane domain includes a transmembrane domain of Fas;
the extracellular domain includes an extracellular domain of Fas, the intracellular domain includes an intracellular domain of CD30, and the transmembrane domain includes a transmembrane domain of CD30;
the extracellular domain includes an extracellular domain of Fas, the intracellular domain includes an intracellular domain of DR3, and the transmembrane domain includes a transmembrane domain of DR3;
the extracellular domain includes an extracellular domain of Fas, the intracellular domain includes an intracellular domain of LIGHT, and the transmembrane domain includes a transmembrane domain of Fas;
the extracellular domain includes an extracellular domain of CD200R, the intracellular domain includes an intracellular domain of CD2, and the transmembrane domain includes a transmembrane domain of CD28;
the extracellular domain includes an extracellular domain of CD200R, the intracellular domain includes an intracellular domain of CD226, and the transmembrane domain includes a transmembrane domain of CD28;
the extracellular domain includes an extracellular domain of CD200R, the intracellular domain includes an intracellular domain of CRTAM, and the transmembrane domain includes a transmembrane domain of CD28;
the extracellular domain includes an extracellular domain of CD200R, the intracellular domain includes an intracellular domain of HAVCR1, and the transmembrane domain includes a transmembrane domain of CD28;
the extracellular domain includes an extracellular domain of CD200R, the intracellular domain includes an intracellular domain of SLAMF3, and the transmembrane domain includes a transmembrane domain of CD28;
the extracellular domain includes an extracellular domain of CD200R, the intracellular domain includes an intracellular domain of SLAMF5, and the transmembrane domain includes a transmembrane domain of CD28;
the extracellular domain includes an extracellular domain of CD200R, the intracellular domain includes an intracellular domain of SLAMF7, and the transmembrane domain includes a transmembrane domain of CD28;
the extracellular domain includes an extracellular domain of SIRPα, the intracellular domain includes an intracellular domain of CD2, and the transmembrane domain includes a transmembrane domain of CD28;
the extracellular domain includes an extracellular domain of SIRPα, the intracellular domain includes an intracellular domain of CD226, and the transmembrane domain includes a transmembrane domain of CD28;
the extracellular domain includes an extracellular domain of SIRPα, the intracellular domain includes an intracellular domain of CRTAM, and the transmembrane domain includes a transmembrane domain of CD28;
the extracellular domain includes an extracellular domain of SIRPα, the intracellular domain includes an intracellular domain of HAVCR1, and the transmembrane domain includes a transmembrane domain of CD28;
the extracellular domain includes an extracellular domain of SIRPα, the intracellular domain includes an intracellular domain of SLAMF3, and the transmembrane domain includes a transmembrane domain of CD28;
the extracellular domain includes an extracellular domain of SIRPα, the intracellular domain includes an intracellular domain of SLAMF5, and the transmembrane domain includes a transmembrane domain of CD28; or
the extracellular domain includes an extracellular domain of SIRPα, the intracellular domain includes an intracellular domain of SLAMF7, and the transmembrane domain includes a transmembrane domain of CD28.
5. The protein of embodiment 1, wherein the intracellular domain includes the sequence as set forth in SEQ ID NO: 38, SEQ ID NO: 39, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 40, or SEQ ID NO: 41
or a sequence having at least 90% or 95% sequence identity to the sequence as set forth in SEQ ID NO: 38, SEQ ID NO: 39, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 40, or SEQ ID NO: 41.
6. The protein of any of embodiments 1-5, having the sequence as set forth in SEQ ID NO: 65, SEQ ID NO: 69, SEQ ID NO: 64, SEQ ID NO: 66, SEQ ID NO: 67, SEQ ID NO: 68, SEQ ID NO: 70, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 71, SEQ ID NO: 72, SEQ ID NO: 73, SEQ ID NO: 74, SEQ ID NO: 75, SEQ ID NO: 76, or SEQ ID NO: 77
or a sequence having at least 90% or 95% sequence identity to the sequence as set forth in SEQ ID NO: 65, SEQ ID NO: 69, SEQ ID NO: 64, SEQ ID NO: 66, SEQ ID NO: 67, SEQ ID NO: 68, SEQ ID NO: 70, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 71, SEQ ID NO: 72, SEQ ID NO: 73, SEQ ID NO: 74, SEQ ID NO: 75, SEQ ID NO: 76, or SEQ ID NO: 77.
7. A protein including
an extracellular domain of Fas, CD200R, or SIRPα; an intracellular domain of ICOS, SLAMF1, CD27, CD40, GITR, or OX40; and a transmembrane domain linking the extracellular domain to the intracellular domain.
8. The protein of embodiment 7, wherein
the extracellular domain includes an extracellular domain of Fas, the intracellular domain includes an intracellular domain of CD27, and the transmembrane domain includes a transmembrane domain of Fas;
the extracellular domain includes an extracellular domain of Fas, the intracellular domain includes an intracellular domain of CD27, and the transmembrane domain includes a transmembrane domain of CD27;
the extracellular domain includes an extracellular domain of Fas, the intracellular domain includes an intracellular domain of CD40, and the transmembrane domain includes a transmembrane domain of Fas;
the extracellular domain includes an extracellular domain of Fas, the intracellular domain includes an intracellular domain of CD40, and the transmembrane domain includes a transmembrane domain of CD40;
the extracellular domain includes an extracellular domain of Fas, the intracellular domain includes an intracellular domain of GITR, and the transmembrane domain includes a transmembrane domain of Fas;
the extracellular domain includes an extracellular domain of Fas, the intracellular domain includes an intracellular domain of GITR, and the transmembrane domain includes a transmembrane domain of GITR;
the extracellular domain includes an extracellular domain of Fas, the intracellular domain includes an intracellular domain of OX40, and the transmembrane domain includes a transmembrane domain of Fas;
the extracellular domain includes an extracellular domain of Fas, the intracellular domain includes an intracellular domain of OX40, and the transmembrane domain includes a transmembrane domain of OX40;
the extracellular domain includes an extracellular domain of CD200R, the intracellular domain includes an intracellular domain of ICOS, and the transmembrane domain includes a transmembrane domain of CD28;
the extracellular domain includes an extracellular domain of CD200R, the intracellular domain includes an intracellular domain of SLAMF1, and the transmembrane domain includes a transmembrane domain of CD28;
the extracellular domain includes an extracellular domain of SIRPα, the intracellular domain includes an intracellular domain of ICOS, and the transmembrane domain includes a transmembrane domain of CD28; or
the extracellular domain includes an extracellular domain of SIRPα, the intracellular domain includes an intracellular domain of SLAMF1, and the transmembrane domain includes a transmembrane domain of CD28.
9. The protein of embodiments 7 or 8, having the sequence as set forth in SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 97, SEQ ID NO: 98, SEQ ID NO: 99, SEQ ID NO: 100, SEQ ID NO: 109, or SEQ ID NO: 110
or a sequence having at least 90% or 95% sequence identity to the sequence as set forth in SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 97, SEQ ID NO: 98, SEQ ID NO: 99, SEQ ID NO: 100, SEQ ID NO: 109, or SEQ ID NO: 110.
10. The protein of any of embodiments 1-10, wherein the transmembrane domain includes the transmembrane domain of Fas, HVEM, CD28, 4-1BB, CD27, CD30, CD40, DR3, GITR, or OX40.
11. The protein of any of embodiments 1-3, 5, 7, 9 or 10, wherein the transmembrane domain includes the sequence as set forth in SEQ ID NO: 11, SEQ ID NO: 12, SEQ ID NO: 19, SEQ ID NO: 9, SEQ ID NO: 10, SEQ ID NO: 13, SEQ ID NO: 14, SEQ ID NO: 15, SEQ ID NO: 16, SEQ ID NO: 17, SEQ ID NO: 18, or SEQ ID NO: 20
or a sequence having at least 90% or 95% sequence identity to the sequence as set forth in SEQ ID NO: 11, SEQ ID NO: 12, SEQ ID NO: 19, SEQ ID NO: 9, SEQ ID NO: 10, SEQ ID NO: 13, SEQ ID NO: 14, SEQ ID NO: 15, SEQ ID NO: 16, SEQ ID NO: 17, SEQ ID NO: 18, or SEQ ID NO: 20.
12. The protein of any of embodiments 1-3, 5, 7, 9 or 10, wherein the transmembrane domain is encoded by the sequence as set forth in SEQ ID NO: 8 or a sequence having at least 90% or 95% sequence identity to the sequence as set forth in SEQ ID NO: 8.
13. The protein of any of embodiments 1-12, wherein the intracellular domain of the stimulatory immune cell protein is inverted.
14. The protein of embodiment 13, wherein the inverted intracellular domain is LIGHT.
15. The protein of any of embodiments 1-14, wherein expression of the protein by an immune cell causes activation of the immune cell in the presence of a ligand, whereas an immune cell that does not express the protein has an inhibitory response in the presence of the same ligand.
16. The protein of any of embodiments 1-15, wherein the Fas is an huFas or an muFas.
17. The protein of any of embodiments 1-15, wherein the CD200R is an huCD200R or an muCD200R.
18. The protein of any of embodiments 1-15, wherein the SIRPα is an huSIRPα.
19. The protein of embodiment 16, wherein the Fas includes the sequence as set forth in SEQ ID NO: 5 or SEQ ID NO: 6 or a sequence having at least 90% or 95% sequence identity to the sequence as set forth in SEQ ID NO: 5 or SEQ ID NO: 6.
20. The protein of embodiment 17, wherein the huCD200R includes the sequence as set forth in SEQ ID NO: 3 or SEQ ID NO: 4 or a sequence having at least 90% or 95% sequence identity to the sequence as set forth in SEQ ID NO: 3 or SEQ ID NO: 4.
21. The protein of embodiment 17, wherein the muCD200R includes the sequence as set forth in SEQ ID NO: 2 or a sequence having at least 90% or 95% sequence identity to the sequence as set forth in SEQ ID NO: 2.
22. The protein of embodiment 17, wherein the muCD200R is encoded by the sequence as set forth in SEQ ID NO: 1 or a sequence having at least 90% or 95% sequence identity to the sequence as set forth in SEQ ID NO: 1.
23. The protein of embodiment 18, wherein the SIRPα includes the sequence as set forth in SEQ ID NO: 7 a sequence having at least 90% or 95% sequence identity to the sequence as set forth in SEQ ID NO: 7.
24. The protein of any of embodiments 1-23, further including junction amino acids.
25. The protein of embodiment 24, wherein the junction amino acids include the sequence as set forth in SEQ ID NO: 78, SEQ ID NO: 79, or SEQ ID NO: 80
or a sequence having at least 90% or 95% sequence identity to the sequence as set forth in SEQ ID NO: 78, SEQ ID NO: 79, or SEQ ID NO: 80.
26. The protein of any of embodiments 1-25, further including at least a second intracellular domain, the at least second intracellular domain also linked to the extracellular domain through the transmembrane domain.
27. The protein of any of embodiments 1-26, wherein the protein further includes a multimerization domain.
28. The protein of embodiment 27, wherein the multimerization domain is part of a chemically-induced dimerization system or a chemically-induced trimerization system.
29. A nucleotide including a gene encoding a protein of any of embodiments 1-28.
30. The nucleotide of embodiment 29, including a second gene encoding a second protein of any of embodiments 1-28.
31. The nucleotide of embodiment 30, wherein the gene encodes Fas-HVEM and the second gene encodes Fas-CD40, Fas-CD27, or SIRPα-ICOS.
32. The nucleotide of embodiment 30, wherein the gene encodes Fas-CD40 and the second gene encodes Fas-HVEM, Fas-CD27, or SIRPα-ICOS.
33. The nucleotide of embodiment 30, wherein the gene encodes Fas-CD27 and the second gene encodes Fas-HVEM, Fas-CD40, or SIRPα-ICOS.
34. The nucleotide of embodiment 30, wherein the gene encodes SIRPα-ICOS and the second gene encodes Fas-CD40, Fas-CD27, or Fas-HVEM.
35. The nucleotide of any of embodiments 30-34, including a third gene encoding a third protein of any of embodiments 1-28.
36. The nucleotide of any of embodiments 29-35, wherein the nucleotide includes regulatory elements including an inducible promoter.
37. The nucleotide of embodiment 30, wherein the first gene and the second gene are under the regulatory control of different inducible promoters.
38. The nucleotide of embodiment 35, wherein the first gene, the second gene, and the third gene are under the regulatory control of different inducible promoters.
39. A cell genetically modified to express a protein of any of embodiments 1-28.
40. A cell of embodiment 39, genetically modified to express a second protein of any of embodiments 1-28.
41. The cell of embodiment 40, wherein the first protein includes Fas-HVEM and the second protein includes Fas-CD40, Fas-CD27, or SIRPα-ICOS.
42. The cell of embodiment 40, wherein the first protein includes Fas-CD40 and the second protein includes Fas-HVEM, Fas-CD27, or SIRPα-ICOS.
43. The cell of embodiment 40, wherein the first protein includes Fas-CD27 and the second protein includes Fas-HVEM, Fas-CD40, or SIRPα-ICOS.
44. The cell of embodiment 40, wherein the first protein includes SIRPα-ICOS and the protein includes encodes Fas-CD40, Fas-CD27, or Fas-HVEM.
45. The cell of any of embodiments 39-44, genetically modified to express a third protein of any of embodiments 1-28.
46. The cell of any of embodiments 39-45, wherein the cell is an immune cell.
47. The cell of embodiment 46, wherein the immune cell is a T cell or a natural killer (NK) cell.
48. The cell of embodiment 47, wherein the T cell is a CD4+ or a CD8+ T cell.
49. The cell of any of embodiments 39-45, wherein the cell is an induced pluripotent stem cell (iPSC), a tumor-infiltrating lymphocyte (TIL), a marrow-infiltrating lymphocyte (MIL), a natural killer T cell (NKT), a mucosal-associated invariant T (MAIT) cell, a B cell, a dendritic cell, a monocyte or a macrophage.
50. A method of genetically-modifying an immune cell to express a protein of any of embodiments 1-28 including introducing a nucleotide of any of embodiments 29-38 into the immune cell.
51. A formulation including a cell of any of embodiments 39-49 and a pharmaceutically acceptable carrier.
52. A nanoparticle including a nucleotide of any of embodiments 29-38.
53. The nanoparticle of embodiment 52, further including a cell targeting ligand.
54. The nanoparticle of embodiment 53, wherein the cell targeting ligand binds a T cell surface marker.
55. The nanoparticle of embodiment 54, wherein the T cell surface marker is CD3, CD4, or CD8.
56. The nanoparticle of any of embodiments 52-55, wherein the cell targeting ligand binds an iPSC marker, a TIL marker, a MIL marker, an NKT marker, a MAIT cell marker, a B cell marker, a dendritic cell marker, a monocyte marker, or a macrophage marker.
57. A composition including a nanoparticle of any of embodiments 52-56 and a pharmaceutically acceptable carrier.
58. A method of stimulating an immune response in a subject in need thereof including administering a therapeutically effective amount of any of embodiments 29-38, a formulation of embodiment 51, or a nanoparticle of any of embodiments 52-56 to the subject, thereby stimulating the immune response in the subject thereof.
59. The method of embodiment 58, wherein the stimulating increases stimulatory intracellular signaling in immune cells within the subject in the presence of inhibitory signals.
60. The method of embodiment 59, wherein the increased stimulatory intracellular signaling in immune cells within the subject results in increased immune cell proliferation and/or increased immune cell activation in the subject, as compared to a comparable subject who was not administered the therapeutically effective amount.
61. The method of any of embodiments 58-60, wherein the stimulating provides an anti-cancer effect or an anti-infection effect in the subject.

(viii) Closing Paragraphs. The nucleic acid and amino acid sequences provided herein are shown using letter abbreviations for nucleotide bases and amino acid residues, as defined in 37 C.F.R. § 1.822 and set forth in the tables in WIPO Standard ST.25 (1998), Appendix 2, Tables 1 and 3. Only one strand of each nucleic acid sequence is shown, but the complementary strand is understood as included in embodiments where it would be appropriate.

To the extent not explicitly provided herein, coding sequences for proteins disclosed herein and protein sequences for coding sequences disclosed herein can be readily derived from one of ordinary skill in the art.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W. H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157 (1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0+1); glutamate (+3.0+1); Ser (+0.3); Asn (+0.2); Gln (+0.2); Gly (0); Thr (−0.4); Pro (−0.5+1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within +2 is preferred, those within +1 are particularly preferred, and those within +0.5 are even more particularly preferred.

As outlined above, amino acid substitutions may be based on the relative similarity of the amino acid side-chain substituents, for example, their hydrophobicity, hydrophilicity, charge, size, and the like.

As indicated elsewhere, variants of gene sequences can include codon optimized variants, sequence polymorphisms, splice variants, and/or mutations that do not affect the function of an encoded product to a statistically significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including (but not limited to) those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure, it will be understood that where sequence analysis software is used for analysis, the results of the analysis are based on the “default values” of the program referenced. As used herein “default values” will mean any set of values or parameters, which originally load with the software when first initialized.

“Specifically binds” refers to an association of a binding domain (of, for example, a recombinant protein disclosed herein) to its cognate binding molecule with an affinity or Kₐ (i.e., an equilibrium association constant of a particular binding interaction with units of 1/M) equal to or greater than 10⁵ M⁻¹, while not significantly associating with any other molecules or components in a relevant environment sample. Binding domains may be classified as “high affinity” or “low affinity”. In particular embodiments, “high affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding domains refer to those binding domains with a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M). In certain embodiments, a binding domain may have “enhanced affinity,” which refers to a selected or engineered binding domains with stronger binding to a cognate binding molecule than a wild type (or parent) binding domain. For example, enhanced affinity may be due to a Kₐ (equilibrium association constant) for the cognate binding molecule that is higher than the reference binding domain or due to a K_{d} (dissociation constant) for the cognate binding molecule that is less than that of the reference binding domain, or due to an off-rate (K_{off}) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N. Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to obtain a claimed effect according to a relevant experimental method described in the current disclosure.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of 35 20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A protein comprising
an extracellular domain of Fas, CD200R, or SIRPα;
an intracellular domain of HVEM, CD2, CD226, CRTAM, HAVCR1, SLAMF3, SLAMF5, SLAMF7, DR3, CD30, or LIGHT; and
a transmembrane domain linking the extracellular domain to the intracellular domain.

2. The protein of claim 1, wherein
the extracellular domain is an extracellular domain of Fas and the intracellular domain is an intracellular domain of HVEM, CD30, DR3, LIGHT, CD2, CD226, CRTAM, HAVCR1, SLAMF3, SLAMF5, or SLAMF7;
the extracellular domain is an extracellular domain of CD200R and the intracellular domain is an intracellular domain of CD2, CD226, CRTAM, HAVCR1, SLAMF3, SLAMF5, SLAMF7, CD30, DR3, HVEM, or LIGHT; or
the extracellular domain is an extracellular domain of SIRPα and the intracellular domain is an intracellular domain of CD2, CD226, CRTAM, HAVCR1, SLAMF3, SLAMF5, SLAMF7, CD30, DR3, HVEM, or LIGHT.

3. The protein of claim 1, wherein
the extracellular domain comprises an extracellular domain of Fas and the intracellular domain comprises an intracellular domain of HVEM;
the extracellular domain comprises an extracellular domain of Fas and the intracellular domain comprises an intracellular domain of CD2;
the extracellular domain comprises an extracellular domain of Fas and the intracellular domain comprises an intracellular domain of CD226;
the extracellular domain comprises an extracellular domain of Fas and the intracellular domain comprises an intracellular domain of CRTAM;
the extracellular domain comprises an extracellular domain of Fas and the intracellular domain comprises an intracellular domain of HAVCR1;
the extracellular domain comprises an extracellular domain of Fas and the intracellular domain comprises an intracellular domain of SLAMF3;
the extracellular domain comprises an extracellular domain of Fas and the intracellular domain comprises an intracellular domain of SLAMF5;
the extracellular domain comprises an extracellular domain of Fas and the intracellular domain comprises an intracellular domain of SLAMF7;
the extracellular domain comprises an extracellular domain of Fas and the intracellular domain comprises an intracellular domain of CD30;
the extracellular domain comprises an extracellular domain of Fas and the intracellular domain comprises an intracellular domain of DR3;
the extracellular domain comprises an extracellular domain of Fas and the intracellular domain comprises an intracellular domain of LIGHT;
the extracellular domain comprises an extracellular domain of CD200R and the intracellular domain comprises an intracellular domain of CD2;
the extracellular domain comprises an extracellular domain of CD200R and the intracellular domain comprises an intracellular domain of CD226;
the extracellular domain comprises an extracellular domain of CD200R and the intracellular domain comprises an intracellular domain of CRTAM;
the extracellular domain comprises an extracellular domain of CD200R and the intracellular domain comprises an intracellular domain of HAVCR1;
the extracellular domain comprises an extracellular domain of CD200R and the intracellular domain comprises an intracellular domain of SLAMF3;
the extracellular domain comprises an extracellular domain of CD200R and the intracellular domain comprises an intracellular domain of SLAMF5;
the extracellular domain comprises an extracellular domain of CD200R and the intracellular domain comprises an intracellular domain of SLAMF7;
the extracellular domain comprises an extracellular domain of CD200R and the intracellular domain comprises an intracellular domain of CD30;
the extracellular domain comprises an extracellular domain of CD200R and the intracellular domain comprises an intracellular domain of DR3;
the extracellular domain comprises an extracellular domain of CD200R and the intracellular domain comprises an intracellular domain of HVEM;
the extracellular domain comprises an extracellular domain of CD200R and the intracellular domain comprises an intracellular domain of LIGHT;
the extracellular domain comprises an extracellular domain of SIRPα and the intracellular domain comprises an intracellular domain of CD2;
the extracellular domain comprises an extracellular domain of SIRPα and the intracellular domain comprises an intracellular domain of CD226;
the extracellular domain comprises an extracellular domain of SIRPα and the intracellular domain comprises an intracellular domain of CRTAM;
the extracellular domain comprises an extracellular domain of SIRPα and the intracellular domain comprises an intracellular domain of HAVCR1;
the extracellular domain comprises an extracellular domain of SIRPα and the intracellular domain comprises an intracellular domain of SLAMF3;
the extracellular domain comprises an extracellular domain of SIRPα and the intracellular domain comprises an intracellular domain of SLAMF5;
the extracellular domain comprises an extracellular domain of SIRPα and the intracellular domain comprises an intracellular domain of SLAMF7;
the extracellular domain comprises an extracellular domain of SIRPα and the intracellular domain comprises an intracellular domain of CD30;
the extracellular domain comprises an extracellular domain of SIRPα and the intracellular domain comprises an intracellular domain of DR3;
the extracellular domain comprises an extracellular domain of SIRPα and the intracellular domain comprises an intracellular domain of HVEM; or the extracellular domain comprises an extracellular domain of SIRPα and the intracellular domain comprises an intracellular domain of LIGHT.

4. The protein of claim 1, wherein
the extracellular domain comprises an extracellular domain of Fas, the intracellular domain comprises an intracellular domain of HVEM, and the transmembrane domain comprises a transmembrane domain of Fas;
the extracellular domain comprises an extracellular domain of Fas, the intracellular domain comprises an intracellular domain of HVEM, and the transmembrane domain comprises a transmembrane domain of HVEM;
the extracellular domain comprises an extracellular domain of Fas, the intracellular domain comprises an intracellular domain of CD30, and the transmembrane domain comprises a transmembrane domain of Fas;
the extracellular domain comprises an extracellular domain of Fas, the intracellular domain comprises an intracellular domain of CD30, and the transmembrane domain comprises a transmembrane domain of CD30;
the extracellular domain comprises an extracellular domain of Fas, the intracellular domain comprises an intracellular domain of DR3, and the transmembrane domain comprises a transmembrane domain of DR3;
the extracellular domain comprises an extracellular domain of Fas, the intracellular domain comprises an intracellular domain of LIGHT, and the transmembrane domain comprises a transmembrane domain of Fas;
the extracellular domain comprises an extracellular domain of CD200R, the intracellular domain comprises an intracellular domain of CD2, and the transmembrane domain comprises a transmembrane domain of CD28;
the extracellular domain comprises an extracellular domain of CD200R, the intracellular domain comprises an intracellular domain of CD226, and the transmembrane domain comprises a transmembrane domain of CD28;
the extracellular domain comprises an extracellular domain of CD200R, the intracellular domain comprises an intracellular domain of CRTAM, and the transmembrane domain comprises a transmembrane domain of CD28;
the extracellular domain comprises an extracellular domain of CD200R, the intracellular domain comprises an intracellular domain of HAVCR1, and the transmembrane domain comprises a transmembrane domain of CD28;
the extracellular domain comprises an extracellular domain of CD200R, the intracellular domain comprises an intracellular domain of SLAMF3, and the transmembrane domain comprises a transmembrane domain of CD28;
the extracellular domain comprises an extracellular domain of CD200R, the intracellular domain comprises an intracellular domain of SLAMF5, and the transmembrane domain comprises a transmembrane domain of CD28;
the extracellular domain comprises an extracellular domain of CD200R, the intracellular domain comprises an intracellular domain of SLAMF7, and the transmembrane domain comprises a transmembrane domain of CD28;
the extracellular domain comprises an extracellular domain of SIRPα, the intracellular domain comprises an intracellular domain of CD2, and the transmembrane domain comprises a transmembrane domain of CD28;
the extracellular domain comprises an extracellular domain of SIRPα, the intracellular domain comprises an intracellular domain of CD226, and the transmembrane domain comprises a transmembrane domain of CD28;
the extracellular domain comprises an extracellular domain of SIRPα, the intracellular domain comprises an intracellular domain of CRTAM, and the transmembrane domain comprises a transmembrane domain of CD28;
the extracellular domain comprises an extracellular domain of SIRPα, the intracellular domain comprises an intracellular domain of HAVCR1, and the transmembrane domain comprises a transmembrane domain of CD28;
the extracellular domain comprises an extracellular domain of SIRPα, the intracellular domain comprises an intracellular domain of SLAMF3, and the transmembrane domain comprises a transmembrane domain of CD28;
the extracellular domain comprises an extracellular domain of SIRPα, the intracellular domain comprises an intracellular domain of SLAMF5, and the transmembrane domain comprises a transmembrane domain of CD28; or
the extracellular domain comprises an extracellular domain of SIRPα, the intracellular domain comprises an intracellular domain of SLAMF7, and the transmembrane domain comprises a transmembrane domain of CD28.

5. The protein of claim 1, wherein the intracellular domain comprises the sequence as set forth in SEQ ID NO: 38, SEQ ID NO: 39, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 40, or SEQ ID NO: 41
or a sequence having at least 90% or 95% sequence identity to the sequence as set forth in SEQ ID NO: 38, SEQ ID NO: 39, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 40, or SEQ ID NO: 41.

6. The protein of claim 1, having the sequence as set forth in SEQ ID NO: 65, SEQ ID NO: 69, SEQ ID NO: 64, SEQ ID NO: 66, SEQ ID NO: 67, SEQ ID NO: 68, SEQ ID NO: 70, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 71, SEQ ID NO: 72, SEQ ID NO: 73, SEQ ID NO: 74, SEQ ID NO: 75, SEQ ID NO: 76, or SEQ ID NO: 77
or a sequence having at least 90% or 95% sequence identity to the sequence as set forth in SEQ ID NO: 65, SEQ ID NO: 69, SEQ ID NO: 64, SEQ ID NO: 66, SEQ ID NO: 67, SEQ ID NO: 68, SEQ ID NO: 70, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 71, SEQ ID NO: 72, SEQ ID NO: 73, SEQ ID NO: 74, SEQ ID NO: 75, SEQ ID NO: 76, or SEQ ID NO: 77.

7. A protein comprising
an extracellular domain of Fas, CD200R, or SIRPα;
an intracellular domain of CD40, ICOS, SLAMF1, CD27, GITR, or OX40; and
a transmembrane domain linking the extracellular domain to the intracellular domain.

8. The protein of claim 7, wherein
the extracellular domain comprises an extracellular domain of Fas, the intracellular domain comprises an intracellular domain of CD27, and the transmembrane domain comprises a transmembrane domain of Fas;
the extracellular domain comprises an extracellular domain of Fas, the intracellular domain comprises an intracellular domain of CD27, and the transmembrane domain comprises a transmembrane domain of CD27;
the extracellular domain comprises an extracellular domain of Fas, the intracellular domain comprises an intracellular domain of CD40, and the transmembrane domain comprises a transmembrane domain of Fas;
the extracellular domain comprises an extracellular domain of Fas, the intracellular domain comprises an intracellular domain of CD40, and the transmembrane domain comprises a transmembrane domain of CD40;
the extracellular domain comprises an extracellular domain of Fas, the intracellular domain comprises an intracellular domain of GITR, and the transmembrane domain comprises a transmembrane domain of Fas;
the extracellular domain comprises an extracellular domain of Fas, the intracellular domain comprises an intracellular domain of GITR, and the transmembrane domain comprises a transmembrane domain of GITR;
the extracellular domain comprises an extracellular domain of Fas, the intracellular domain comprises an intracellular domain of OX40, and the transmembrane domain comprises a transmembrane domain of Fas;
the extracellular domain comprises an extracellular domain of Fas, the intracellular domain comprises an intracellular domain of OX40, and the transmembrane domain comprises a transmembrane domain of OX40;
the extracellular domain comprises an extracellular domain of CD200R, the intracellular domain comprises an intracellular domain of ICOS, and the transmembrane domain comprises a transmembrane domain of CD28;
the extracellular domain comprises an extracellular domain of CD200R, the intracellular domain comprises an intracellular domain of SLAMF1, and the transmembrane domain comprises a transmembrane domain of CD28;
the extracellular domain comprises an extracellular domain of SIRPα, the intracellular domain comprises an intracellular domain of ICOS, and the transmembrane domain comprises a transmembrane domain of CD28; or
the extracellular domain comprises an extracellular domain of SIRPα, the intracellular domain comprises an intracellular domain of SLAMF1, and the transmembrane domain comprises a transmembrane domain of CD28.

9. The protein of claim 7, having the sequence as set forth in SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 97, SEQ ID NO: 98, SEQ ID NO: 99, SEQ ID NO: 100, SEQ ID NO: 109, or SEQ ID NO: 110
or a sequence having at least 90% or 95% sequence identity to the sequence as set forth in SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 97, SEQ ID NO: 98, SEQ ID NO: 99, SEQ ID NO: 100, SEQ ID NO: 109, or SEQ ID NO: 110.

10. The protein of claim 1, wherein the transmembrane domain comprises the transmembrane domain of Fas, HVEM, CD28, 4-1BB, CD27, CD30, CD40, DR3, GITR, or OX40.

11. The protein of claim 1, wherein the transmembrane domain comprises the sequence as set forth in SEQ ID NO: 11, SEQ ID NO: 12, SEQ ID NO: 19, SEQ ID NO: 9, SEQ ID NO: 10, SEQ ID NO: 13, SEQ ID NO: 14, SEQ ID NO: 15, SEQ ID NO: 16, SEQ ID NO: 17, SEQ ID NO: 18, or SEQ ID NO: 20
or a sequence having at least 90% or 95% sequence identity to the sequence as set forth in SEQ ID NO: 11, SEQ ID NO: 12, SEQ ID NO: 19, SEQ ID NO: 9, SEQ ID NO: 10, SEQ ID NO: 13, SEQ ID NO: 14, SEQ ID NO: 15, SEQ ID NO: 16, SEQ ID NO: 17, SEQ ID NO: 18, or SEQ ID NO: 20.

12. The protein of claim 1, wherein the transmembrane domain is encoded by the sequence as set forth in SEQ ID NO: 8 or a sequence having at least 90% or 95% sequence identity to the sequence as set forth in SEQ ID NO: 8.

13. The protein of claim 1, wherein the intracellular domain of the stimulatory immune cell protein is inverted.

14. The protein of claim 13, wherein the inverted intracellular domain is LIGHT.

15. The protein of claim 1, wherein expression of the protein by an immune cell causes activation of the immune cell in the presence of a ligand, whereas an immune cell that does not express the protein has an inhibitory response in the presence of the same ligand.

16. The protein of claim 1, wherein the Fas is an huFas or an muFas.

17. The protein of claim 1, wherein the CD200R is an huCD200R or an muCD200R.

18. The protein of claim 1, wherein the SIRPα is an huSIRPα.

19. The protein of claim 16, wherein the Fas comprises the sequence as set forth in SEQ ID NO: 5 or SEQ ID NO: 6 or a sequence having at least 90% or 95% sequence identity to the sequence as set forth in SEQ ID NO: 5 or SEQ ID NO: 6.

20. The protein of claim 17, wherein the huCD200R comprises the sequence as set forth in SEQ ID NO: 3 or SEQ ID NO: 4 or a sequence having at least 90% or 95% sequence identity to the sequence as set forth in SEQ ID NO: 3 or SEQ ID NO: 4.

21. The protein of claim 17, wherein the muCD200R comprises the sequence as set forth in SEQ ID NO: 2 or a sequence having at least 90% or 95% sequence identity to the sequence as set forth in SEQ ID NO: 2.

22. The protein of claim 17, wherein the muCD200R is encoded by the sequence as set forth in SEQ ID NO: 1 or a sequence having at least 90% or 95% sequence identity to the sequence as set forth in SEQ ID NO: 1.

23. The protein of claim 18, wherein the SIRPα comprises the sequence as set forth in SEQ ID NO: 7 a sequence having at least 90% or 95% sequence identity to the sequence as set forth in SEQ ID NO: 7.

24. The protein of claim 1, further comprising junction amino acids.

25. The protein of claim 24, wherein the junction amino acids comprise the sequence as set forth in SEQ ID NO: 78, SEQ ID NO: 79, or SEQ ID NO: 80
or a sequence having at least 90% or 95% sequence identity to the sequence as set forth in SEQ ID NO: 78, SEQ ID NO: 79, or SEQ ID NO: 80.

26. The protein of claim 1, further comprising at least a second intracellular domain, the at least second intracellular domain also linked to the extracellular domain through the transmembrane domain.

27. The protein of claim 1, wherein the protein further comprises a multimerization domain.

28. The protein of claim 27, wherein the multimerization domain is part of a chemically-induced dimerization system or a chemically-induced trimerization system.

29. A nucleotide comprising a gene encoding a protein of claim 1.

30. The nucleotide of claim 29, comprising a second gene encoding a second protein of any of claim 1.

31. The nucleotide of claim 30, wherein the gene encodes Fas-HVEM and the second gene encodes Fas-CD40, Fas-CD27, or SIRPα-ICOS.

32. The nucleotide of claim 30, wherein the gene encodes Fas-CD40 and the second gene encodes Fas-HVEM, Fas-CD27, or SIRPα-ICOS.

33. The nucleotide of claim 30, wherein the gene encodes Fas-CD27 and the second gene encodes Fas-HVEM, Fas-CD40, or SIRPα-ICOS.

34. The nucleotide of claim 30, wherein the gene encodes SIRPα-ICOS and the second gene encodes Fas-CD40, Fas-CD27, or Fas-HVEM.

35. The nucleotide of claim 30, comprising a third gene encoding a third protein of claim 1.

36. The nucleotide of claim 29, wherein the nucleotide comprises regulatory elements comprising an inducible promoter.

37. The nucleotide of claim 30, wherein the first gene and the second gene are under the regulatory control of different inducible promoters.

38. The nucleotide of claim 35, wherein the first gene, the second gene, and the third gene are under the regulatory control of different inducible promoters.

39. A cell genetically modified to express a protein of claim 1.

40. A cell of claim 39, genetically modified to express a second protein of claim 1.

41. The cell of claim 40, wherein the first protein comprises Fas-HVEM and the second protein comprises Fas-CD40, Fas-CD27, or SIRPα-ICOS.

42. The cell of claim 40, wherein the first protein comprises Fas-CD40 and the second protein comprises Fas-HVEM, Fas-CD27, or SIRPα-ICOS.

43. The cell of claim 40, wherein the first protein comprises Fas-CD27 and the second protein comprises Fas-HVEM, Fas-CD40, or SIRPα-ICOS.

44. The cell of claim 40, wherein the first protein comprises SIRPα-ICOS and the protein comprises encodes Fas-CD40, Fas-CD27, or Fas-HVEM.

45. The cell of claim 39, genetically modified to express a third protein of claim 1.

46. The cell of claim 39, wherein the cell is an immune cell.

47. The cell of claim 39, wherein the cell is a T cell or a natural killer (NK) cell.

48. The cell of claim 47, wherein the T cell is a CD4+ or a CD8+ T cell.

49. The cell of claim 39, wherein the cell is an induced pluripotent stem cell (iPSC), a tumor-infiltrating lymphocyte (TIL), a marrow-infiltrating lymphocyte (MIL), a natural killer T cell (NKT), a mucosal-associated invariant T (MAIT) cell, a B cell, a dendritic cell, a monocyte or a macrophage.

50. A method of genetically-modifying an immune cell to express a protein of claim 1 comprising introducing a nucleotide of claim 29 into the immune cell.

51. A formulation comprising a cell of claim 39 and a pharmaceutically acceptable carrier.

52. A nanoparticle comprising a nucleotide of claim 29.

53. The nanoparticle of claim 52, further comprising a cell targeting ligand.

54. The nanoparticle of claim 53, wherein the cell targeting ligand binds a T cell surface marker.

55. The nanoparticle of claim 54, wherein the T cell surface marker is CD3, CD4, or CD8.

56. The nanoparticle of claim 53, wherein the cell targeting ligand binds an iPSC marker, a TIL marker, a MIL marker, an NKT marker, a MAIT cell marker, a B cell marker, a dendritic cell marker, a monocyte marker, or a macrophage marker.

57. A composition comprising a nanoparticle of claim 52 and a pharmaceutically acceptable carrier.

58. A method of stimulating an immune response in a subject in need thereof comprising administering a therapeutically effective amount of a nucleotide of claim 29, a formulation of claim 51, or a nanoparticle of claim 52 to the subject, thereby stimulating the immune response in the subject thereof.

59. The method of claim 58, wherein the stimulating increases stimulatory intracellular signaling in immune cells within the subject in the presence of inhibitory signals.

60. The method of claim 59, wherein the increased stimulatory intracellular signaling in immune cells within the subject results in increased immune cell proliferation and/or increased immune cell activation in the subject, as compared to a comparable subject who was not administered the therapeutically effective amount.

61. The method of claim 58, wherein the stimulating provides an anti-cancer effect or an anti-infection effect in the subject.


SEQUENCE LISTING (110 sequences)

SEQ ID NO: 1 - DNA, 687 bp, Mus musculus
  source 1..687: mol_type = genomic DNA
atgttctgcttctggcggacaagcgccctggccgtgctgctgatctggggagtgtttgtggccggcagcagctgcaccgacaagaaccagaccacccagaacaacagcagcagccccctgacccaagtgaacaccaccgtgtccgtgcagatcggcaccaaggccctgctgtgctgtttcagcatccctctgaccaaggctgtgctgatcacctggatcatcaagctgagaggcctgcccagctgcacaatcgcctacaaggtggacaccaagaccaacgagacaagctgcctgggcagaaacatcacctgggccagcaccccagaccacagccctgagctgcagatcagcgccgtgacactgcagcacgagggcacctacacatgcgagacagtgacccccgagggcaacttcgagaagaactacgatctgcaggtgctggtgccccccgaagtgacctacttccccgagaagaatagaagcgccgtgtgcgaggccatggctggcaaacctgccgcccagatctcttggagccctgacggcgactgtgtgaccaccagcgagagccacagcaacggcacagtgaccgtgcggagcacctgtcactgggagcagaacaacgtgtccgacgtgtcctgcatcgtgtcccacctgaccggcaaccagagcctgagcatcgagctgagc

SEQ ID NO: 2 - protein, 200 aa, Mus musculus
MFCFWRTSALAVLLIWGVFVAGSSCTDKNQTTQNNSSSPLTQVNTTVSVQIGTKALLCCFSIPLTKAVLITWIIKLRGLPSCTIAYKVDTKTNETSCLGRNITWASTPDHSPELQISAVTLQHEGTYTCETVTPEGNFEKNYDLQVLVPPEVTYFPEKNRSAVCEAMAGKPAAQISWSPDGDCVTTSESHSNGTVTVRST

SEQ ID NO: 3 - protein, 243 aa, Homo sapiens
MLCPWRTANLGLLLILTIFLVAASSSLCMDEKQITQNYSKVLAEVNTSWPVKMATNAVLCCPPIALRNLIIITWEIILRGQPSCTKAYKKETNETKETNCTDERITWVSRPDQNSDLQIRTVAITHDGYYRCIMVTPDGNFHRGYHLQVLVTPEVTLFQNRNRTAVCKAVAGKPAAHISWIPEGDCATKQEYWSNGTVTVKSTCHWEVHNVSTVTCHVSHLTGNKSLYIELCPSPLFPGPSKP

SEQ ID NO: 4 - protein, 231 aa, Homo sapiens
MLCPWRTANLGLLLILTIFLVAASSSLCMDEKQITQNYSKVLAEVNTSWPVKMATNAVLCCPPIALRNLIIITWEIILRGQPSCTKAYKKETNETKETNCTDERITWVSRPDQNSDLQIRTVAITHDGYYRCIMVTPDGNFHRGYHLQVLVTPEVTLFQNRNRTAVCKAVAGKPAAHISWIPEGDCATKQEYWSNGTVTVKSTCHWEVHNVSTVTCHVSHLTGNKSLYIEL

SEQ ID NO: 5 - protein, 169 aa, Mus musculus
MLWIWAVLPLVLAGSQLRVHTQGTNSISESLKLRRRVRETDKNCSEGLYQGGPFCCQPCQPGKKKVEDCKMNGGTPTCAPCTEGKEYMDKNHYADKCRRCTLCDEEHGLEVETNCTLTQNTKCKCKPDFYCDSPGCEHCVRCASCEHGTLEPCTATSNTNCRKQSPRNR

SEQ ID NO: 6 - protein, 173 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSN

SEQ ID NO: 7 - protein, 361 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTA

SEQ ID NO: 8 - DNA, 108 bp, Mus musculus
  source 1..108: mol_type = genomic DNA
tgccacacccagagcagccccaagctgttctgggccctggtggtggtggccggcgtgctgttttgttacggcctgctcgtgaccgtggccctgtgcgtgatctggacc

SEQ ID NO: 9 - protein, 36 aa, Mus musculus
CHTQSSPKLFWALVVVAGVLFCYGLLVTVALCVIWT

SEQ ID NO: 10 - protein, 27 aa, Homo sapiens
FWVLVVVGGVLACYSLLVTVAFIIFWV

SEQ ID NO: 11 - protein, 17 aa, Homo sapiens
LGWLCLLLLPIPLIVWV

SEQ ID NO: 12 - protein, 17 aa, Mus musculus
LWLLTILVLLIPLVFIY

SEQ ID NO: 13 - protein, 21 aa, Mus musculus
TLFLALTSALLLALIFITLLF

SEQ ID NO: 14 - protein, 21 aa, Mus musculus
IFVTFSSMFLIFVLGAILFFH

SEQ ID NO: 15 - protein, 21 aa, Mus musculus
PVLFWVAMVVLLVGSGSFLLC

SEQ ID NO: 16 - protein, 22 aa, Mus musculus
ALLVIPVVMGILITIFGVFLYI

SEQ ID NO: 17 - protein, 22 aa, Mus musculus
FWVQVLLGVAFLFGAILICAYC

SEQ ID NO: 18 - protein, 21 aa, Mus musculus
LTVIFLVMAACIFFLTTVQLG

SEQ ID NO: 19 - protein, 21 aa, Mus musculus
VVSILLPLVIVGAGIAGFLIC

SEQ ID NO: 20 - protein, 25 aa, Mus musculus
AFAVLLGLGLGLLAPLTVLLALYLL

SEQ ID NO: 21 - protein, 116 aa, Homo sapiens
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 22 - protein, 115 aa, Mus musculus
KRRKRNRRRKDEELEIKASRTSTVERGPKPHSTPAAAAQNSVALQAPPPPGHHLQTPGHRPLPPGHRTREHQQKKRPPPSGTQIHQQKGPPLPRPRVQPKPPCGSGDGVSLPPPN

SEQ ID NO: 23 - protein, 61 aa, Homo sapiens
NRRRRRERRDLFTESWDTQKAPNNYRSPISTSQPTNQSMDDTREDIYVNYPTFSRRPKTRV

SEQ ID NO: 24 - protein, 58 aa, Mus musculus
NRKRRRQVRIPLKEPRDKQSKVATNCRSPTSPIQSTDDEKEDIYVNYPTFSRRPKPRL

SEQ ID NO: 25 - protein, 85 aa, Homo sapiens
IMKLRKAHVIWKKENEVSEHTLESYRSRSNNEETSSEEKNGQSSHPMRCMNYITKLYSEAKTKRKENVQHSKLEEKHIQVPESIV

SEQ ID NO: 26 - protein, 83 aa, Mus musculus
IMKLRKAHVVWKKESEISEQALESYRSRSNNEETSSQENSSQAPQSKRCMNYITRLYSGAKTKKSAQHWKLGGKHSRVPESIV

SEQ ID NO: 27 - protein, 48 aa, Homo sapiens
KKYFFKKEVQQLSVSFSSLQIKALQNAVEKEVQAEDNIYIENSLYATD

SEQ ID NO: 28 - protein, 47 aa, Mus musculus
TRYILMKRKSASLSVVAFRVSKIEALQNAAVVHSRAEDNIYIVEDRP

SEQ ID NO: 29 - protein, 179 aa, Homo sapiens
KRKGRCSVPAFCSSQAEAPADTPEPTAGHTLYSVLSQGYEKLDTPLRPARQQPTPTSDSSSDSNLTTEEDEDRPEVHKPISGRYEVFDQVTQEGAGHDPAPEGQADYDPVTPYVTEVESVVGENTMYAQVFNLQGKTPVSQKEESSATIYCSIRKPQVVPPPQQNDLEIPESPTYENFT

SEQ ID NO: 30 - protein, 180 aa, Mus musculus
RKKKQCSSLATRYRQAEVPAEIPETPTGHGQFSVLSQRYEKLDMSAKTTRHQPTPTSDTSSESSATTEEDDEKTRMHSTANSRNQVYDLVTHQDIAHALAYEGQVEYEAITPYDKVDGSMDEEDMAYIQVSLNVQGETPLPQKKEDSNTIYCSVQKPKKTAQTPQQDAESPETPTYENFT

SEQ ID NO: 31 - protein, 99 aa, Homo sapiens
RLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVI

SEQ ID NO: 32 - protein, 87 aa, Mus musculus
RLYKRRRDRIVLEADDVSKKTVYAVVSRNAQPTESRIYDEIPQSKMLSCKEDPVTTIYSSVQLSEKMKETNMKDRSLPKALGNEIVV

SEQ ID NO: 33 - protein, 88 aa, Homo sapiens
WFLKRERQEEYIEEKKRVDICRETPNICPHSGENTEYDTIPHTNRTILKEDPANTVYSTVEIPKKMENPHSLLTMPDTPRLFAYENVI

SEQ ID NO: 34 - protein, 88 aa, Mus musculus
HTTWIKKGKGCEEDKKRVDRHQEMPDLCPHLEENADYDTIPYTEKRRPEEDAPNTFYSTVQIPKVVKSPSSLPAKPLVPRSLSFENVI

SEQ ID NO: 35 - protein, 189 aa, Homo sapiens
CHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 36 - protein, 190 aa, Mus musculus
YWKACRRRFQQKFHLDYLVQTFQPKMEQTDSCPTEKLTQPQRSGSVTDPSTGHKLSPVSPPPAVETCASVGATYLENLPLLDDSPAGNPFSPREPPEPRVSTEHTNNRIEKIYIMKADTVIVGSVKTEVPEGRAPAGSTESELEAELEVDHAPHYPEQETEPPLGSCTEVMFSVEEGGKEDHGPTTVSEK

SEQ ID NO: 37 - protein, 198 aa, Mus musculus
ICAYCRWQPCKAVVTADTAGTETLASPQTAHLSASDSAHTLLAPPSSTGKICTTVQLVGNNWTPGLSQTQEVVCGQASQPWDQLPNRTLGTPLASPLSPAPPAGSPAAVLQPGPQLYDVMDAVPARRWKEFVRTLGLREAEIEAVEVEICRFRDQQYEMLKRWRQQQPAGLGAIYAALERMGLEGCAEDLRSRLQRGP

SEQ ID NO: 38 - protein, 60 aa, Homo sapiens
CVKRRKPRGDVVKVIVSVQRKRQEAEGEATVIEALQAPPDVTTVAVEETIPSFTGRSPNH

SEQ ID NO: 39 - protein, 44 aa, Mus musculus
TRRHLHTSSVAKELEPFQEQQENTIRFPVTEVGFAETEEETASN

SEQ ID NO: 40 - protein, 37 aa, Homo sapiens
MEESVVRPSVFVVDGQTDIPFTRLGRSHRRQSCSVAR

SEQ ID NO: 41 - protein, 37 aa, Mus musculus
MESVVQPSVFVVDGQTDIPFRRLEQNHRRRRCGTVQV

SEQ ID NO: 42 - protein, 12 aa, Homo sapiens
CPSPLFPGPSKP

SEQ ID NO: 43 - protein, 325 aa, Homo sapiens
MLCPWRTANLGLLLILTIFLVAASSSLCMDEKQITQNYSKVLAEVNTSWPVKMATNAVLCCPPIALRNLIIITWEIILRGQPSCTKAYKKETNETKETNCTDERITWVSRPDQNSDLQIRTVAITHDGYYRCIMVTPDGNFHRGYHLQVLVTPEVTLFQNRNRTAVCKAVAGKPAAHISWIPEGDCATKQEYWSNGTVTVKSTCHWEVHNVSTVTCHVSHLTGNKSLYIELLPVPGAKKSAKLYIPYIILTIIILTIVGFIWLLKVNGCRKYKLNKTESTPVVEEDEMQPYASYTEKNNPLYDTTNKVKASEALQSEVDTDLHTL

SEQ ID NO: 44 - protein, 186 aa, Mus musculus
MLWIWAVLPLVLAGSQLRVHTQGTNSISESLKLRRRVRETDKNCSEGLYQGGPFCCQPCQPGKKKVEDCKMNGGTPTCAPCTEGKEYMDKNHYADKCRRCTLCDEEHGLEVETNCTLTQNTKCKCKPDFYCDSPGCEHCVRCASCEHGTLEPCTATSNTNCRKQSPRNRLWLLTILVLLIPLVFIY

SEQ ID NO: 45 - protein, 190 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWV

SEQ ID NO: 46 - protein, 394 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLV

SEQ ID NO: 47 - protein, 236 aa, Mus musculus
MFCFWRTSALAVLLIWGVFVAGSSCTDKNQTTQNNSSSPLTQVNTTVSVQIGTKALLCCFSIPLTKAVLITWIIKLRGLPSCTIAYKVDTKTNETSCLGRNITWASTPDHSPELQISAVTLQHEGTYTCETVTPEGNFEKNYDLQVLVPPEVTYFPEKNRSAVCEAMAGKPAAQISWSPDGDCVTTSESHSNGTVTVRSTCHTQSSPKLFWALVVVAGVLFCYGLLVTVALCVIWT

SEQ ID NO: 48 - protein, 270 aa, Homo sapiens
MLCPWRTANLGLLLILTIFLVAASSSLCMDEKQITQNYSKVLAEVNTSWPVKMATNAVLCCPPIALRNLIIITWEIILRGQPSCTKAYKKETNETKETNCTDERITWVSRPDQNSDLQIRTVAITHDGYYRCIMVTPDGNFHRGYHLQVLVTPEVTLFQNRNRTAVCKAVAGKPAAHISWIPEGDCATKQEYWSNGTVTVKSTCHWEVHNVSTVTCHVSHLTGNKSLYIELCPSPLFPGPSKPFWVLVVVGGVLACYSLLVTVAFIIFWV

SEQ ID NO: 49 - protein, 400 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTACPSPLFPGPSKPFWVLVVVGGVLACYSLLVTVAFIIFWV

SEQ ID NO: 50 - protein, 386 aa, Homo sapiens
MLCPWRTANLGLLLILTIFLVAASSSLCMDEKQITQNYSKVLAEVNTSWPVKMATNAVLCCPPIALRNLIIITWEIILRGQPSCTKAYKKETNETKETNCTDERITWVSRPDQNSDLQIRTVAITHDGYYRCIMVTPDGNFHRGYHLQVLVTPEVTLFQNRNRTAVCKAVAGKPAAHISWIPEGDCATKQEYWSNGTVTVKSTCHWEVHNVSTVTCHVSHLTGNKSLYIELCPSPLFPGPSKPFWVLVVVGGVLACYSLLVTVAFIIFWVKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 51 - protein, 331 aa, Homo sapiens
MLCPWRTANLGLLLILTIFLVAASSSLCMDEKQITQNYSKVLAEVNTSWPVKMATNAVLCCPPIALRNLIIITWEIILRGQPSCTKAYKKETNETKETNCTDERITWVSRPDQNSDLQIRTVAITHDGYYRCIMVTPDGNFHRGYHLQVLVTPEVTLFQNRNRTAVCKAVAGKPAAHISWIPEGDCATKQEYWSNGTVTVKSTCHWEVHNVSTVTCHVSHLTGNKSLYIELCPSPLFPGPSKPFWVLVVVGGVLACYSLLVTVAFIIFWVNRRRRRERRDLFTESWDTQKAPNNYRSPISTSQPTNQSMDDTREDIYVNYPTFSRRPKTRV

SEQ ID NO: 52 - protein, 355 aa, Homo sapiens
MLCPWRTANLGLLLILTIFLVAASSSLCMDEKQITQNYSKVLAEVNTSWPVKMATNAVLCCPPIALRNLIIITWEIILRGQPSCTKAYKKETNETKETNCTDERITWVSRPDQNSDLQIRTVAITHDGYYRCIMVTPDGNFHRGYHLQVLVTPEVTLFQNRNRTAVCKAVAGKPAAHISWIPEGDCATKQEYWSNGTVTVKSTCHWEVHNVSTVTCHVSHLTGNKSLYIELCPSPLFPGPSKPFWVLVVVGGVLACYSLLVTVAFIIFWVIMKLRKAHVIWKKENEVSEHTLESYRSRSNNEETSSEEKNGQSSHPMRCMNYITKLYSEAKTKRKENVQHSKLEEKHIQVPESIV

SEQ ID NO: 53 - protein, 318 aa, Homo sapiens
MLCPWRTANLGLLLILTIFLVAASSSLCMDEKQITQNYSKVLAEVNTSWPVKMATNAVLCCPPIALRNLIIITWEIILRGQPSCTKAYKKETNETKETNCTDERITWVSRPDQNSDLQIRTVAITHDGYYRCIMVTPDGNFHRGYHLQVLVTPEVTLFQNRNRTAVCKAVAGKPAAHISWIPEGDCATKQEYWSNGTVTVKSTCHWEVHNVSTVTCHVSHLTGNKSLYIELCPSPLFPGPSKPFWVLVVVGGVLACYSLLVTVAFIIFWVKKYFFKKEVQQLSVSFSSLQIKALQNAVEKEVQAEDNIYIENSLYATD

SEQ ID NO: 54 - protein, 449 aa, Homo sapiens
MLCPWRTANLGLLLILTIFLVAASSSLCMDEKQITQNYSKVLAEVNTSWPVKMATNAVLCCPPIALRNLIIITWEIILRGQPSCTKAYKKETNETKETNCTDERITWVSRPDQNSDLQIRTVAITHDGYYRCIMVTPDGNFHRGYHLQVLVTPEVTLFQNRNRTAVCKAVAGKPAAHISWIPEGDCATKQEYWSNGTVTVKSTCHWEVHNVSTVTCHVSHLTGNKSLYIELCPSPLFPGPSKPFWVLVVVGGVLACYSLLVTVAFIIFWVKRKGRCSVPAFCSSQAEAPADTPEPTAGHTLYSVLSQGYEKLDTPLRPARQQPTPTSDSSSDSNLTTEEDEDRPEVHKPISGRYEVFDQVTQEGAGHDPAPEGQADYDPVTPYVTEVESVVGENTMYAQVFNLQGKTPVSQKEESSATIYCSIRKPQVVPPPQQNDLEIPESPTYENFT

SEQ ID NO: 55 - protein, 369 aa, Homo sapiens
MLCPWRTANLGLLLILTIFLVAASSSLCMDEKQITQNYSKVLAEVNTSWPVKMATNAVLCCPPIALRNLIIITWEIILRGQPSCTKAYKKETNETKETNCTDERITWVSRPDQNSDLQIRTVAITHDGYYRCIMVTPDGNFHRGYHLQVLVTPEVTLFQNRNRTAVCKAVAGKPAAHISWIPEGDCATKQEYWSNGTVTVKSTCHWEVHNVSTVTCHVSHLTGNKSLYIELCPSPLFPGPSKPFWVLVVVGGVLACYSLLVTVAFIIFWVRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVI

SEQ ID NO: 56 - protein, 358 aa, Homo sapiens
MLCPWRTANLGLLLILTIFLVAASSSLCMDEKQITQNYSKVLAEVNTSWPVKMATNAVLCCPPIALRNLIIITWEIILRGQPSCTKAYKKETNETKETNCTDERITWVSRPDQNSDLQIRTVAITHDGYYRCIMVTPDGNFHRGYHLQVLVTPEVTLFQNRNRTAVCKAVAGKPAAHISWIPEGDCATKQEYWSNGTVTVKSTCHWEVHNVSTVTCHVSHLTGNKSLYIELCPSPLFPGPSKPFWVLVVVGGVLACYSLLVTVAFIIFWVWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 57 - protein, 351 aa, Mus musculus
MFCFWRTSALAVLLIWGVFVAGSSCTDKNQTTQNNSSSPLTQVNTTVSVQIGTKALLCCFSIPLTKAVLITWIIKLRGLPSCTIAYKVDTKTNETSCLGRNITWASTPDHSPELQISAVTLQHEGTYTCETVTPEGNFEKNYDLQVLVPPEVTYFPEKNRSAVCEAMAGKPAAQISWSPDGDCVTTSESHSNGTVTVRSTCHTQSSPKLFWALVVVAGVLFCYGLLVTVALCVIWTKRRKRNRRRKDEELEIKASRTSTVERGPKPHSTPAAAAQNSVALQAPPPPGHHLQTPGHRPLPPGHRTREHQQKKRPPPSGTQIHQQKGPPLPRPRVQPKPPCGSGDGVSLPPPN

SEQ ID NO: 58 - protein, 294 aa, Mus musculus
MFCFWRTSALAVLLIWGVFVAGSSCTDKNQTTQNNSSSPLTQVNTTVSVQIGTKALLCCFSIPLTKAVLITWIIKLRGLPSCTIAYKVDTKTNETSCLGRNITWASTPDHSPELQISAVTLQHEGTYTCETVTPEGNFEKNYDLQVLVPPEVTYFPEKNRSAVCEAMAGKPAAQISWSPDGDCVTTSESHSNGTVTVRSTCHTQSSPKLFWALVVVAGVLFCYGLLVTVALCVIWTNRKRRRQVRIPLKEPRDKQSKVATNCRSPTSPIQSTDDEKEDIYVNYPTFSRRPKPRL

SEQ ID NO: 59 - protein, 319 aa, Mus musculus
MFCFWRTSALAVLLIWGVFVAGSSCTDKNQTTQNNSSSPLTQVNTTVSVQIGTKALLCCFSIPLTKAVLITWIIKLRGLPSCTIAYKVDTKTNETSCLGRNITWASTPDHSPELQISAVTLQHEGTYTCETVTPEGNFEKNYDLQVLVPPEVTYFPEKNRSAVCEAMAGKPAAQISWSPDGDCVTTSESHSNGTVTVRSTCHTQSSPKLFWALVVVAGVLFCYGLLVTVALCVIWTIMKLRKAHVVWKKESEISEQALESYRSRSNNEETSSQENSSQAPQSKRCMNYITRLYSGAKTKKSAQHWKLGGKHSRVPESIV

SEQ ID NO: 60 - protein, 283 aa, Mus musculus
MFCFWRTSALAVLLIWGVFVAGSSCTDKNQTTQNNSSSPLTQVNTTVSVQIGTKALLCCFSIPLTKAVLITWIIKLRGLPSCTIAYKVDTKTNETSCLGRNITWASTPDHSPELQISAVTLQHEGTYTCETVTPEGNFEKNYDLQVLVPPEVTYFPEKNRSAVCEAMAGKPAAQISWSPDGDCVTTSESHSNGTVTVRSTCHTQSSPKLFWALVVVAGVLFCYGLLVTVALCVIWTTRYILMKRKSASLSVVAFRVSKIEALQNAAVVHSRAEDNIYIVEDRP

SEQ ID NO: 61 - protein, 416 aa, Mus musculus
MFCFWRTSALAVLLIWGVFVAGSSCTDKNQTTQNNSSSPLTQVNTTVSVQIGTKALLCCFSIPLTKAVLITWIIKLRGLPSCTIAYKVDTKTNETSCLGRNITWASTPDHSPELQISAVTLQHEGTYTCETVTPEGNFEKNYDLQVLVPPEVTYFPEKNRSAVCEAMAGKPAAQISWSPDGDCVTTSESHSNGTVTVRSTCHTQSSPKLFWALVVVAGVLFCYGLLVTVALCVIWTRKKKQCSSLATRYRQAEVPAEIPETPTGHGQFSVLSQRYEKLDMSAKTTRHQPTPTSDTSSESSATTEEDDEKTRMHSTANSRNQVYDLVTHQDIAHALAYEGQVEYEAITPYDKVDGSMDEEDMAYIQVSLNVQGETPLPQKKEDSNTIYCSVQKPKKTAQTPQQDAESPETPTYENFT

SEQ ID NO: 62 - protein, 323 aa, Mus musculus
MFCFWRTSALAVLLIWGVFVAGSSCTDKNQTTQNNSSSPLTQVNTTVSVQIGTKALLCCFSIPLTKAVLITWIIKLRGLPSCTIAYKVDTKTNETSCLGRNITWASTPDHSPELQISAVTLQHEGTYTCETVTPEGNFEKNYDLQVLVPPEVTYFPEKNRSAVCEAMAGKPAAQISWSPDGDCVTTSESHSNGTVTVRSTCHTQSSPKLFWALVVVAGVLFCYGLLVTVALCVIWTRLYKRRRDRIVLEADDVSKKTVYAVVSRNAQPTESRIYDEIPQSKMLSCKEDPVTTIYSSVQLSEKMKETNMKDRSLPKALGNEIVV

SEQ ID NO: 63 - protein, 324 aa, Mus musculus
MFCFWRTSALAVLLIWGVFVAGSSCTDKNQTTQNNSSSPLTQVNTTVSVQIGTKALLCCFSIPLTKAVLITWIIKLRGLPSCTIAYKVDTKTNETSCLGRNITWASTPDHSPELQISAVTLQHEGTYTCETVTPEGNFEKNYDLQVLVPPEVTYFPEKNRSAVCEAMAGKPAAQISWSPDGDCVTTSESHSNGTVTVRSTCHTQSSPKLFWALVVVAGVLFCYGLLVTVALCVIWTHTTWIKKGKGCEEDKKRVDRHQEMPDLCPHLEENADYDTIPYTEKRRPEEDAPNTFYSTVQIPKVVKSPSSLPAKPLVPRSLSFENVI

SEQ ID NO: 64 - protein, 379 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 65 - protein, 250 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVCVKRRKPRGDVVKVIVSVQRKRQEAEGEATVIEALQAPPDVTTVAVEETIPSFTGRSPNH

SEQ ID NO: 66 - protein, 227 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVMEESVVRPSVFVVDGQTDIPFTRLGRSHRRQSCSVAR

SEQ ID NO: 67 - protein, 380 aa, Mus musculus
MLWIWAVLPLVLAGSQLRVHTQGTNSISESLKLRRRVRETDKNCSEGLYQGGPFCCQPCQPGKKKVEDCKMNGGTPTCAPCTEGKEYMDKNHYADKCRRCTLCDEEHGLEVETNCTLTQNTKCKCKPDFYCDSPGCEHCVRCASCEHGTLEPCTATSNTNCRKQSPRNRPVLFWVAMVVLLVGSGSFLLCYWKACRRRFQQKFHLDYLVQTFQPKMEQTDSCPTEKLTQPQRSGSVTDPSTGHKLSPVSPPPAVETCASVGATYLENLPLLDDSPAGNPFSPREPPEPRVSTEHTNNRIEKIYIMKADTVIVGSVKTEVPEGRAPAGSTESELEAELEVDHAPHYPEQETEPPLGSCTEVMFSVEEGGKEDHGPTTVSEK

SEQ ID NO: 68 - protein, 389 aa, Mus musculus
MLWIWAVLPLVLAGSQLRVHTQGTNSISESLKLRRRVRETDKNCSEGLYQGGPFCCQPCQPGKKKVEDCKMNGGTPTCAPCTEGKEYMDKNHYADKCRRCTLCDEEHGLEVETNCTLTQNTKCKCKPDFYCDSPGCEHCVRCASCEHGTLEPCTATSNTNCRKQSPRNRFWVQVLLGVAFLFGAILICAYCICAYCRWQPCKAVVTADTAGTETLASPQTAHLSASDSAHTLLAPPSSTGKICTTVQLVGNNWTPGLSQTQEVVCGQASQPWDQLPNRTLGTPLASPLSPAPPAGSPAAVLQPGPQLYDVMDAVPARRWKEFVRTLGLREAEIEAVEVEICRFRDQQYEMLKRWRQQQPAGLGAIYAALERMGLEGCAEDLRSRLQRGP

SEQ ID NO: 69 - protein, 234 aa, Mus musculus
MLWIWAVLPLVLAGSQLRVHTQGTNSISESLKLRRRVRETDKNCSEGLYQGGPFCCQPCQPGKKKVEDCKMNGGTPTCAPCTEGKEYMDKNHYADKCRRCTLCDEEHGLEVETNCTLTQNTKCKCKPDFYCDSPGCEHCVRCASCEHGTLEPCTATSNTNCRKQSPRNRVVSILLPLVIVGAGIAGFLICTRRHLHTSSVAKELEPFQEQQENTIRFPVTEVGFAETEEETASN

SEQ ID NO: 70 - protein, 223 aa, Mus musculus
MLWIWAVLPLVLAGSQLRVHTQGTNSISESLKLRRRVRETDKNCSEGLYQGGPFCCQPCQPGKKKVEDCKMNGGTPTCAPCTEGKEYMDKNHYADKCRRCTLCDEEHGLEVETNCTLTQNTKCKCKPDFYCDSPGCEHCVRCASCEHGTLEPCTATSNTNCRKQSPRNRLWLLTILVLLIPLVFIYMESVVQPSVFVVDGQTDIPFRRLEQNHRRRRCGTVQV

SEQ ID NO: 71 - protein, 516 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTACPSPLFPGPSKPFWVLVVVGGVLACYSLLVTVAFIIFWVKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 72 - protein, 461 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTACPSPLFPGPSKPFWVLVVVGGVLACYSLLVTVAFIIFWVNRRRRRERRDLFTESWDTQKAPNNYRSPISTSQPTNQSMDDTREDIYVNYPTFSRRPKTRV

SEQ ID NO: 73 - protein, 485 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTACPSPLFPGPSKPFWVLVVVGGVLACYSLLVTVAFIIFWVIMKLRKAHVIWKKENEVSEHTLESYRSRSNNEETSSEEKNGQSSHPMRCMNYITKLYSEAKTKRKENVQHSKLEEKHIQVPESIV

SEQ ID NO: 74 - protein, 448 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTACPSPLFPGPSKPFWVLVVVGGVLACYSLLVTVAFIIFWVKKYFFKKEVQQLSVSFSSLQIKALQNAVEKEVQAEDNIYIENSLYATD

SEQ ID NO: 75 - protein, 579 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTACPSPLFPGPSKPFWVLVVVGGVLACYSLLVTVAFIIFWVKRKGRCSVPAFCSSQAEAPADTPEPTAGHTLYSVLSQGYEKLDTPLRPARQQPTPTSDSSSDSNLTTEEDEDRPEVHKPISGRYEVFDQVTQEGAGHDPAPEGQADYDPVTPYVTEVESVVGENTMYAQVFNLQGKTPVSQKEESSATIYCSIRKPQVVPPPQQNDLEIPESPTYENFT

SEQ ID NO: 76 - protein, 499 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTACPSPLFPGPSKPFWVLVVVGGVLACYSLLVTVAFIIFWVRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVI

SEQ ID NO: 77 - protein, 488 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTACPSPLFPGPSKPFWVLVVVGGVLACYSLLVTVAFIIFWVWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 78-80 - skipped (3 entries)

SEQ ID NO: 81 - protein, 41 aa, Homo sapiens
RSKRSRLLHSDYMNMTPRRPGPTRKHYQPYAPPRDFAAYRS

SEQ ID NO: 82 - protein, 41 aa, Homo sapiens
NSRRNRLLQSDYMNMTPRRPGLTRKPYQPYAPARDFAAYRP

SEQ ID NO: 83 - protein, 38 aa, Homo sapiens
CWLTKKKYSSSVHDPNGEYMFMRAVNTAKKSRLTDVTL

SEQ ID NO: 84 - protein, 35 aa, Mus musculus
SKKKYGSSVHDPNSEYMFMAAVNTNKKSRLAGVTS

SEQ ID NO: 85 - protein, 77 aa, Homo sapiens
QLRRRGKTNHYQTTVEKKSLTIYAQVQKPGPLQKKLDSFPAQDPCTTIYVAATEPVPESVQETNSITVYASVTLPES

SEQ ID NO: 86 - protein, 78 aa, Mus musculus
MMKRQGKSNHCQPPVEEKSLTIYAQVQKSGPQEKKLHDALTDQDPCTTIYVAATEPAPESVQEPNPTTVYASVTLPES

SEQ ID NO: 87 - protein, 42 aa, Homo sapiens
KRGRKKLLYIFKQPFMRPVQTTQEEDGCSCRFPEEEEGGCEL

SEQ ID NO: 88 - protein, 48 aa, Mus musculus
SVLKWIRKKFPHIFKQPFKKTTGAAQEEDACSCRCPQEEEGGGGGYEL

SEQ ID NO: 89 - protein, 48 aa, Homo sapiens
QRRKYRSNKGESPVEPAEPCHYSCPREEEGSTIPIQEDYRKPEPACSP

SEQ ID NO: 90 - protein, 47 aa, Mus musculus
QRRNHGPNEDRQAVPEEPCPYSCPREEEGSAIPIQEDYRKPEPAFYP

SEQ ID NO: 91 - protein, 62 aa, Homo sapiens
KKVAKKPTNKAPHPKQEPQEINFPDDLPGSNTAAPVQETLHGCQPVTQEDGKESRISVQERQ

SEQ ID NO: 92 - protein, 74 aa, Mus musculus
KKVVKKPKDNEILPPAARRQDPQEMEDYPGHNTAAPVQETLHGCQPVTQEDGKESRISVQERQVTDSIALRPLV

SEQ ID NO: 93 - protein, 58 aa, Homo sapiens
QLGLHIWQLRSQCMWPRETQLLLEVPPSTEDARSCQFPEEERGERSAEEKGRLGDLWV

SEQ ID NO: 94 - protein, 54 aa, Mus musculus
LHIWQLRRQHMCPRETQPFAEVQLSAEDACSFQFPEEERGEQTEEKCHLGGRWP

SEQ ID NO: 95 - protein, 42 aa, Homo sapiens
ALYLLRRDQRLPPDAHKPPGGGSFRTPIQEEQADAHSTLAKI

SEQ ID NO: 96 - protein, 36 aa, Mus musculus
RKAWRLPNTPKPCWGNSFRTPIQEEHTDAHFTLAKI

SEQ ID NO: 97 - protein, 308 aa, Homo sapiens
MLCPWRTANLGLLLILTIFLVAASSSLCMDEKQITQNYSKVLAEVNTSWPVKMATNAVLCCPPIALRNLIIITWEIILRGQPSCTKAYKKETNETKETNCTDERITWVSRPDQNSDLQIRTVAITHDGYYRCIMVTPDGNFHRGYHLQVLVTPEVTLFQNRNRTAVCKAVAGKPAAHISWIPEGDCATKQEYWSNGTVTVKSTCHWEVHNVSTVTCHVSHLTGNKSLYIELCPSPLFPGPSKPFWVLVVVGGVLACYSLLVTVAFIIFWVCWLTKKKYSSSVHDPNGEYMFMRAVNTAKKSRLTDVTL

SEQ ID NO: 98 - protein, 347 aa, Homo sapiens
MLCPWRTANLGLLLILTIFLVAASSSLCMDEKQITQNYSKVLAEVNTSWPVKMATNAVLCCPPIALRNLIIITWEIILRGQPSCTKAYKKETNETKETNCTDERITWVSRPDQNSDLQIRTVAITHDGYYRCIMVTPDGNFHRGYHLQVLVTPEVTLFQNRNRTAVCKAVAGKPAAHISWIPEGDCATKQEYWSNGTVTVKSTCHWEVHNVSTVTCHVSHLTGNKSLYIELCPSPLFPGPSKPFWVLVVVGGVLACYSLLVTVAFIIFWVQLRRRGKTNHYQTTVEKKSLTIYAQVQKPGPLQKKLDSFPAQDPCTTIYVAATEPVPESVQETNSITVYASVTLPES

SEQ ID NO: 99 - protein, 271 aa, Mus musculus
MFCFWRTSALAVLLIWGVFVAGSSCTDKNQTTQNNSSSPLTQVNTTVSVQIGTKALLCCFSIPLTKAVLITWIIKLRGLPSCTIAYKVDTKTNETSCLGRNITWASTPDHSPELQISAVTLQHEGTYTCETVTPEGNFEKNYDLQVLVPPEVTYFPEKNRSAVCEAMAGKPAAQISWSPDGDCVTTSESHSNGTVTVRSTCHTQSSPKLFWALVVVAGVLFCYGLLVTVALCVIWTSKKKYGSSVHDPNSEYMFMAAVNTNKKSRLAGVTS

SEQ ID NO: 100 - protein, 314 aa, Mus musculus
MFCFWRTSALAVLLIWGVFVAGSSCTDKNQTTQNNSSSPLTQVNTTVSVQIGTKALLCCFSIPLTKAVLITWIIKLRGLPSCTIAYKVDTKTNETSCLGRNITWASTPDHSPELQISAVTLQHEGTYTCETVTPEGNFEKNYDLQVLVPPEVTYFPEKNRSAVCEAMAGKPAAQISWSPDGDCVTTSESHSNGTVTVRSTCHTQSSPKLFWALVVVAGVLFCYGLLVTVALCVIWTMMKRQGKSNHCQPPVEEKSLTIYAQVQKSGPQEKKLHDALTDQDPCTTIYVAATEPAPESVQEPNPTTVYASVTLPES

SEQ ID NO: 101 - protein, 238 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVQRRKYRSNKGESPVEPAEPCHYSCPREEEGSTIPIQEDYRKPEPACSP

SEQ ID NO: 102 - protein, 252 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKKVAKKPTNKAPHPKQEPQEINFPDDLPGSNTAAPVQETLHGCQPVTQEDGKESRISVQERQ

SEQ ID NO: 103 - protein, 248 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVQLGLHIWQLRSQCMWPRETQLLLEVPPSTEDARSCQFPEEERGERSAEEKGRLGDLWV

SEQ ID NO: 104 - protein, 232 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVALYLLRRDQRLPPDAHKPPGGGSFRTPIQEEQADAHSTLAKI

SEQ ID NO: 105 - protein, 237 aa, Mus musculus
MLWIWAVLPLVLAGSQLRVHTQGTNSISESLKLRRRVRETDKNCSEGLYQGGPFCCQPCQPGKKKVEDCKMNGGTPTCAPCTEGKEYMDKNHYADKCRRCTLCDEEHGLEVETNCTLTQNTKCKCKPDFYCDSPGCEHCVRCASCEHGTLEPCTATSNTNCRKQSPRNRIFVTFSSMFLIFVLGAILFFHQRRNHGPNEDRQAVPEEPCPYSCPREEEGSAIPIQEDYRKPEPAFYP

SEQ ID NO: 106 - protein, 265 aa, Mus musculus
MLWIWAVLPLVLAGSQLRVHTQGTNSISESLKLRRRVRETDKNCSEGLYQGGPFCCQPCQPGKKKVEDCKMNGGTPTCAPCTEGKEYMDKNHYADKCRRCTLCDEEHGLEVETNCTLTQNTKCKCKPDFYCDSPGCEHCVRCASCEHGTLEPCTATSNTNCRKQSPRNRALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 107 - protein, 244 aa, Mus musculus
MLWIWAVLPLVLAGSQLRVHTQGTNSISESLKLRRRVRETDKNCSEGLYQGGPFCCQPCQPGKKKVEDCKMNGGTPTCAPCTEGKEYMDKNHYADKCRRCTLCDEEHGLEVETNCTLTQNTKCKCKPDFYCDSPGCEHCVRCASCEHGTLEPCTATSNTNCRKQSPRNRLTVIFLVMAACIFFLTTVQLGLHIWQLRRQHMCPRETQPFAEVQLSAEDACSFQFPEEERGEQTEEKCHLGGRWP

SEQ ID NO: 108 - protein, 230 aa, Mus musculus
MLWIWAVLPLVLAGSQLRVHTQGTNSISESLKLRRRVRETDKNCSEGLYQGGPFCCQPCQPGKKKVEDCKMNGGTPTCAPCTEGKEYMDKNHYADKCRRCTLCDEEHGLEVETNCTLTQNTKCKCKPDFYCDSPGCEHCVRCASCEHGTLEPCTATSNTNCRKQSPRNRAFAVLLGLGLGLLAPLTVLLALYLLRKAWRLPNTPKPCWGNSFRTPIQEEHTDAHFTLAKI

SEQ ID NO: 109 - protein, 438 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTACPSPLFPGPSKPFWVLVVVGGVLACYSLLVTVAFIIFWVCWLTKKKYSSSVHDPNGEYMFMRAVNTAKKSRLTDVTL

SEQ ID NO: 110 - protein, 477 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTACPSPLFPGPSKPFWVLVVVGGVLACYSLLVTVAFIIFWVQLRRRGKTNHYQTTVEKKSLTIYAQVQKPGPLQKKLDSFPAQDPCTTIYVAATEPVPESVQETNSITVYASVTLPES